(19) United States
(12) Patent Application Publication
(10) Pub. No.: US 2018/0125995 A1
(43) Pub. Date: May 10, 2018

(54) ANTI-STAPHYLOCOCCUS AUREUS ANTIBODY RIFAMYCIN CONJUGATES AND USES THEREOF

(71) Applicant: Genentech, Inc., South San Francisco, CA (US)

(72) Inventors: Eric Brown, San Francisco, CA (US); Wouter Hazenbos, San Francisco, CA (US); Isidro Hotzel, Brisbane, CA (US); Kimberly Kajihara, San Francisco, CA (US); Sophie M. Lehar, Montara, CA (US); Sanjeev Mariathasan, Millbrae, CA (US); Thomas Pillow, San Francisco, CA (US); Leanna Staben, San Francisco, CA (US); Vishal Verma, San Carlos, CA (US); Binqing Wei, Belmont, CA (US); Yi Xia, Cupertino, CA (US); Min Xu, South San Francisco, CA (US)

(73) Assignee: Genentech, Inc., South San Francisco, CA (US)

(21) Appl. No.: 15/612,193

(22) Filed: Jun. 2, 2017

Related U.S. Application Data
(63) Continuation of application No. PCT/US2015/063515, filed on Dec. 2, 2015.
(60) Provisional application No. 62/087,213, filed on Dec. 3, 2014.

(51) Int. Cl. A61K 47/68; C07K 16/12; A61K 31/5383; A61K 45/06; A61P 31/04

(52) U.S. Cl. CPC A61K 47/6803; A61K 47/6835; C07K 16/1271; C07K 2317/21; A61K 45/06; A61P 31/04; A61K 31/5383

(57) ABSTRACT

The invention provides rF1 antibody antibiotic conjugates and methods of using same.

### CROSS REFERENCE TO RELATED APPLICATION

This application is a continuation of International Patent Application No. PCT/US2015/063515, having an international filing date of Dec. 2, 2015, the entire contents of which are incorporated herein by reference, and which claims the benefit under 35 U.S.C. § 119(e) to U.S. Provisional Application No. 62/087,213, filed Dec. 3, 2014, which is herein incorporated by reference in its entirety.

### SEQUENCE LISTING

The instant application contains a Sequence Listing which has been submitted electronically in ASCII format and is hereby incorporated by reference in its entirety. Said ASCII copy, created on May 30, 2017, is named P32350-US-1_SequenceListing.txt and is 26,787 bytes in size.

### FIELD OF THE INVENTION

The invention relates to anti-Staphylococcus antibodies conjugated to rifamycin-type antibiotics and to use of the resultant antibody-antibiotic conjugates in the treatment of Staphylococcus infections.

### BACKGROUND OF THE INVENTION

Staphylococcus aureus and S. epidermidis are successful human commensals that primarily colonize the nares and skin. Staphylococcus aureus (S. aureus; SA) can also invade a variety of tissues, leading to life-threatening infections; it is the leading cause of bacterial infections in humans worldwide. Recently emerged strains of S. aureus show increased virulence and enhanced ability to cause disease in otherwise healthy individuals. Over the last several decades, infection with S. aureus has become increasingly difficult to treat due to the emergence and rapid spread of methicillin-resistant S. aureus (MRSA) that is resistant to all known beta-lactam antibiotics (Boucher, H. W., et al. (2009) Clin Infect Dis 48, 1-12). Currently, the most prevalent and most virulent clinical strain of methicillin resistant S. aureus (MRSA) is USA300, which has the capacity to produce a large number of virulence factors and cause mortality in infected individuals (Chambers, H F and Deleo F R (2009) Nature Reviews Microbiology 7:629-641). The most serious infections such as endocarditis, osteomyelitis, necrotizing pneumonia and sepsis occur following dissemination of the bacteria into the bloodstream (Lowy, F. D. (1998) N Engl J Med 339, 520-532). S. epidermidis, which is closely related to S. aureus, is often associated with hospital-acquired infections, and represents the most common source of infections on indwelling medical devices.

Important for staphylococcal adhesion to and successful colonization of host tissues, is a family of cell wall proteins, characterized by a large stretch of serine-aspartate dipeptide (SDR) repeats adjacent to an adhesive A-domain, that is present in staphylococci (Foster T J, Hook M (1998) Trends Microbiol 6: 484-488). Such proteins important for adherence include clumping factor (Clf)A and ClfB (Foster T J, supra). In addition to ClfA and ClfB, S. aureus also expresses three SDR-proteins, SdrC, SdrD and SdrE, which are organized in tandem in the genome. These proteins are also thought to be involved in tissue colonization, and elimination of any of them decreases bacterial virulence (Cheng A G, et al. (2009) FASEB Journal 23: 3393-3404). Three additional members of this family, SrdF, SdrG and SdrH, are present in most S. epidermidis strains (McCrea K W, et al. (2000) The serine-aspartate repeat (Sdr) protein family in Staphylococcus epidermidis. Microbiology 146 (Pt 7): 1535-1546). In each of these proteins, the SDR-region, which contains between 25 and 275 SD-dipeptide repeats (SEQ ID NO: 24), is located between the N-terminal ligand-binding A-domain and a C-terminal LPXTG-motif (SEQ ID NO: 25), which mediates anchoring to the cell wall by the transpeptidase sortase A. The function of the SDR-domain remains unknown, although it has been proposed to act as a cell wall spanning domain allowing exposure of the N terminal ligand binding sites of these proteins (Hartford O, et al. (1997) Mol Microbiol 25: 1065-1076).

It was found that the SDR-domains of all SDR-proteins of S. aureus and S. epidermidis are heavily glycosylated by two novel glycosyltransferases, SdgA and SdgB, which are responsible for glycosylation in two steps (Hazenbos et al. (2013) PLOS Pathogens 9 (10):1-18). These glycosylation events prevent degradation of these proteins by host proteases, thereby preserving bacterial host tissue interactions. Hazenbos et al. (2013) also showed that the SdgB-mediated glycosylation creates an immunodominant epitope for highly opsonic antibodies in humans. These antibodies account for a significant proportion of the total anti-staphylococcal IgG response.

Invasive MRSA infections are hard to treat, with a mortality rate of ˜20% and are the leading cause of death by an infectious agent in the USA. Vancomycin, linezolid and daptomycin have thus become the few antibiotics of choice for treating invasive MRSA infections (Boucher, H., Miller, L. G. & Razonable, R. R. (2010) Clin Infect Dis 51 Suppl 2, S183-197). However, reduced susceptibility to vancomycin and cross-resistance to linezolid and daptomycin have already been reported in MRSA clinical strains (Nannini, E., Murray, B. E. & Arias, C. A. (2010) Curr Opin Pharmacol 10, 516-521). Over time, the vancomycin dose necessary to overcome resistance has crept upward to levels where nephrotoxicity occurs. Thus, mortality and morbidity from invasive MRSA infections remains high despite these antibiotics.

Investigations have revealed that S. aureus is able to invade and survive inside mammalian cells including the phagocytic cells that are responsible for bacterial clearance (Thwaites, G. E. & Gant, V. (2011) Nat Rev Microbiol 9, 215-222); Rogers, D. E., Tompsett, R. (1952) J. Exp. Med 95, 209-230); Gresham, H. D., et al. (2000) J Immunol 164, 3713-3722); Kapral, F. A. & Shayegani, M. G. (1959) J Exp Med 110, 123-138; Anwar, S., et al. (2009) Clin Exp Immunol 157, 216-224); Fraunholz, M. & Sinha, B. (2012) Front Cell Infect Microbiol 2, 43); Garzoni, C. & Kelley, W. L. (2011) EMBO Mol Med 3, 115-117). S. aureus is taken up by host phagocytic cells, primarily neutrophils and macrophages, within minutes following intravenous infection (Rogers, D. E. (1956) JEM 103, 713). While the majority of the bacteria are effectively killed by these cells, incomplete clearance of S. aureus inside blood borne phagocytes can allow these infected cells to act as “Trojan horses” for dissemination of the bacteria away from the initial site of infection. Indeed, patients with normal neutrophil counts may be more prone to disseminated disease than those with reduced neutrophil counts (Thwaites, G. E. & Gant, V. (2011) supra). Once delivered to the tissues, S. aureus can invade various non-phagocytic cell types, and intracellular S. aureus in tissues is associated with chronic or recurrent infections. Furthermore, exposure of intracellular bacteria to suboptimal antibiotic concentrations may encourage the emergence of antibiotic resistant strains, thus making this clinical problem more acute. Consistent with these observations, treatment of patients with invasive MRSA infections such as bacteremia or endocarditis with vancomycin or daptomycin was associated with failure rates greater than 50% (Kullar, R., Davis, S. L., Levine, D. P. & Rybak, M. J. Impact of vancomycin exposure on outcomes in patients with methicillin-resistant Staphylococcus aureus bacteremia: support for consensus guidelines suggested targets. Clinical infectious diseases: an official publication of the Infectious Diseases Society of America 52, 975-981 (2011); Fowler, V. G., Jr. et al. Daptomycin versus standard therapy for bacteremia and endocarditis caused by Staphylococcus aureus. The New England journal of medicine 355, 653-665 (2006); Yoon, Y. K., Kim, J. Y., Park, D. W., Sohn, J. W. & Kim, M. J. Predictors of persistent methicillin-resistant Staphylococcus aureus bacteraemia in patients treated with vancomycin. The Journal of antimicrobial chemotherapy 65:1015-1018 (2010)). Therefore, a more successful anti-staphylococcal therapy should include the elimination of intracellular bacteria.

Ansamycins are a class of antibiotics, including rifamycin, rifampin, rifampicin, rifabutin, rifapentine, rifalazil, ABI-1657, and analogs thereof, that inhibit bacterial RNA polymerase and have exceptional potency against gram-positive and selective gram-negative bacteria (Rothstein, D. M., et al (2003) Expert Opin. Invest. Drugs 12(2):255-271; U.S. Pat. No. 7,342,011; U.S. Pat. No. 7,271,165).

Immunotherapies have been reported for preventing and treating S. aureus (including MRSA) infections. US2011/0262477 concerns uses of bacterial adhesion proteins Eap, Emp and AdsA as vaccines to stimulate immune response against MRSA. WO2000071585 describes isolated monoclonal antibodies reactive to specific S. aureus strain isolates. US20110059085A1 suggests an Ab-based strategy utilizing IgM Abs specific for one or more SA capsular antigens, although no actual antibodies were described.

Antibody-drug conjugates (ADC), also known as immunoconjugates, are targeted chemotherapeutic molecules which combine ideal properties of both antibodies and cytotoxic drugs by targeting potent cytotoxic drugs to antigen-expressing tumor cells (Teicher, B. A. (2009) Curr. Cancer Drug Targets 9:982-1004), thereby enhancing the therapeutic index by maximizing efficacy and minimizing off-target toxicity (Carter, P. J. and Senter P. D. (2008) The Cancer J. 14(3):154-169; Chari, R. V. (2008) Acc. Chem. Res. 41:98-107. ADC comprise a targeting antibody covalently attached through a linker unit to a cytotoxic drug moiety. Immunoconjugates allow for the targeted delivery of a drug moiety to a tumor, and intracellular accumulation therein, where systemic administration of unconjugated drugs may result in unacceptable levels of toxicity to normal cells as well as the tumor cells sought to be eliminated (Polakis P. (2005) Curr. Opin. Pharmacol. 5:382-387).

Non-specific immunoglobulin-antibiotic conjugates are described that bind to the surface of target bacteria via the antibiotic for treating sepsis (U.S. Pat. No. 5,545,721; U.S. Pat. No. 6,660,267). Antibiotic-conjugated antibodies are described that have an antigen-binding portion specific for a bacterial antigen (such as SA capsular polysaccharide), but lack a constant region that reacts with a bacterial Fc-binding protein, e.g., staphylococcal protein A (U.S. Pat. No. 7,569,677).

In view of the alarming rate of resistance of MRSA to conventional antibiotics and the resultant mortality and morbidity from invasive MRSA infections, there is a high unmet need for new therapeutics to treat S. aureus infections. The present invention satisfies this need and by providing compositions and methods that overcome the limitations of current therapeutic compositions as well as offer additional advantages that will be apparent from the detailed description below.

### SUMMARY OF THE INVENTION

The present invention provides a unique therapeutic that includes the elimination of intracellular bacteria. The present invention demonstrates that such a therapeutic is efficacious in-vivo where conventional antibiotics like vancomycin fail.

The invention provides compositions referred to as “antibody-antibiotic conjugates,” or “AAC”) comprising an antibody conjugated by a covalent attachment to one or more rifamycin-type antibiotic moieties.

An aspect of the invention is an antibody-antibiotic conjugate compound comprising an rF1 antibody, covalently attached by a protease-cleavable, non-peptide linker to a rifamycin-type antibiotic.

An exemplary embodiment of the invention is an antibody-antibiotic conjugate having the formula:

Ab-(PM L-abx)ₚ

wherein:

Ab is the rF1 antibody;

PML is the protease-cleavable, non-peptide linker having the formula:

-Str-PM-Y—

where Str is a stretcher unit; PM is a peptidomimetic unit, and Y is a spacer unit;

abx is the rifamycin-type antibiotic; and

p is an integer from 1 to 8.

The antibody-antibiotic conjugate compounds of any of the preceding embodiments can comprise any one of the anti-SDR Abs and specifically rF1 antibodies described herein. These rF1 antibodies bind to Staphylococcus aureus. In exemplary rF1 antibodies, the Ab is a monoclonal antibody comprising a light (L) chain and a heavy (H) chain, the L chain comprising CDR L1, CDR L2, and CDR L3 and the H chain comprising CDR H1, CDR H2 and CDR H3, wherein the CDR H1, CDR H2 and CDR H3 and the CDR L1, CDR L2, and CDR L3 and comprise the amino acid sequences of the CDRs of each of Abs F1 (SEQ ID NO. 1-6), rF1 (SEQ ID NO. 1-5,7), rF1.v1 (SEQ ID NO. 1,8,3,4-6), respectively, as indicated in Tables 4A and 4B.

In some embodiments, the rF1 antibody comprises a heavy chain variable region (VH), wherein the VH comprises at least 95% sequence identity over the length of the VH region selected from the VH sequence of SEQ ID NO. 13. The antibodies may further comprise a L chain variable region (VL) wherein the VL comprises at least 95% sequence identity over the length of the VL region selected from the VL sequence of SEQ ID NO. 14 and SEQ ID NO. 15, of antibodies rF1 and rF1.v6, respectively.

In specific embodiments, the rF1 antibody comprises L and H chain pairs as follows: a L chain comprising the sequence of SEQ ID NO. 9 paired with a H chain comprising the sequence of SEQ ID NO. 10; L chain comprising the sequence of SEQ ID NO. 11 paired with a H chain comprising the sequence of SEQ ID NO. 10; a L chain comprising the sequence of SEQ ID NO. 11 paired with a H chain comprising the sequence of SEQ ID NO. 12.

In any one of the preceding embodiments, the antibody may be an antigen-binding fragment lacking a Fc region. In some embodiments, the antibody is a F(ab) or F(ab′)2. In some embodiments, the antibody further comprises a heavy chain constant region and/or a light chain constant region, wherein the heavy chain constant region and/or the light chain constant region comprise one or more amino acids that are substituted with cysteine residues. In some embodiments, the heavy chain constant region comprises amino acid substitution A118C and/or 5400C, and/or the light chain constant region comprises amino acid substitution V205C, wherein the numbering system is according to EU numbering.

In some embodiments of any of the antibodies described above, the antibody is not an IgM isotype. In some embodiments of any of the antibodies described above, the antibody is an IgG (e.g., IgG1, IgG2, IgG3, IgG4), IgE, IgD, or IgA (e.g., IgA1 or IgA2) isotype.

An exemplary embodiment of the invention is a pharmaceutical composition comprising the antibody-antibiotic conjugate compound, and a pharmaceutically acceptable carrier, glidant, diluent, or excipient.

Another aspect of the invention is a method of treating a bacterial infection comprising administering to an infected patient a therapeutically-effective amount of the antibody-antibiotic conjugate of any of the preceding embodiments. Another aspect of the invention is a method of treating a Staphylococcal infection in a patient comprising administering to the patient a therapeutically-effective amount of an antibody-antibiotic conjugate of the invention. In one embodiment, the patient is a human. In one embodiment the patient is infected with a Staphylococcus aureus and/or a Staphylococcus epidermidis infection. In some embodiments, the patient has been diagnosed with a S. aureus infection. In some embodiments, treating the bacterial infection comprises reducing the bacterial load or counts.

Another aspect of the invention is a method of treating a Staphylococcal infection in an infected patient comprising administering to the patient a therapeutically-effective amount of an antibody-antibiotic conjugate of any one of the preceding embodiments. In one embodiment, the patient is a human. In one embodiment the bacterial infection is a Staphylococcus aureus infection. In some embodiments, the patient has been diagnosed with a S. aureus infection. In some embodiments, treating the bacterial infection comprises reducing the bacterial load or counts.

In one embodiment of any of the preceding methods of treatment, the is administered to patients where the bacterial infection including S. aureus has led to bacteremia. In specific embodiments the method is used to treat Staphylococcal endocarditis or osteomyelitis. In one embodiment, the antibody-antibiotic conjugate compound is administered to the infected patient at a dose in the range of about 50 mg/kg to 100 mg/kg.

Also provided is method of killing intracellular S. aureus in the cells of a S. aureus infected patient without killing the host cells by administering a rF1 antibiotic conjugate compound of any of the above embodiments. Another method is provided for killing persister Staphylococcal bacterial cells (e.g, S. aureus) in vivo by contacting the persister bacteria with an AAC of any of the preceding embodiments.

In another embodiment, the method of treatment further comprises administering a second therapeutic agent. In a further embodiment, the second therapeutic agent is an antibiotic including an antibiotic against Staph aureus in general or MRSA in particular.

In one embodiment, the second antibiotic administered in combination with the antibody-antibiotic conjugate compound of the invention is selected from the structural classes: (i) aminoglycosides; (ii) beta-lactams; (iii) macrolides/cyclic peptides; (iv) tetracyclines; (v) fluoroquinolines/fluoroquinolones; (vi) and oxazolidinones.

In one embodiment, the second antibiotic administered in combination with the antibody-antibiotic conjugate compound of the invention is selected from clindamycin, novobiocin, retapamulin, daptomycin, GSK-2140944, CG-400549, sitafloxacin, teicoplanin, triclosan, napthyridone, radezolid, doxorubicin, ampicillin, vancomycin, imipenem, doripenem, gemcitabine, dalbavancin, and azithromycin.

In some embodiments herein, the bacterial load in the infected patient has been reduced to an undetectable level after the treatment. In one embodiment, the patient's blood culture is negative after treatment as compared to a positive blood culture before treatment. In some embodiments herein, the bacterial resistance in the subject is undetectable or low. In some embodiments herein, the patient is not responsive to treatment with methicillin or vancomycin.

An exemplary embodiment of the invention is a process for making the antibody-antibiotic conjugate comprising conjugating a rifamycin-type antibiotic to an rF1 antibody.

An exemplary embodiment of the invention is a kit for treating a bacterial infection, comprising:

a) the pharmaceutical composition comprising the antibody-antibiotic conjugate compound, and a pharmaceutically acceptable carrier, glidant, diluent, or excipient; and

b) instructions for use.

An aspect of the invention is an antibiotic-linker intermediate having Formula II:

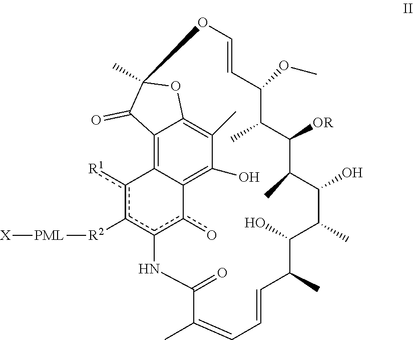

wherein:

the dashed lines indicate an optional bond;

R is H, C₁-C₁₂ alkyl, or C(O)CH₃;

R¹ is OH;

R2 is CH═N-(heterocyclyl), wherein the heterocyclyl is optionally substituted with one or more groups independently selected from C(O)CH3, C1-C12 alkyl, C1-C12 heteroaryl, C2-C20 heterocyclyl, C6-C20 aryl, and C3-C12 carbocyclyl;

or R1 and R2 form a five- or six-membered fused heteroaryl or heterocyclyl, and optionally forming a spiro or fused six-membered heteroaryl, heterocyclyl, aryl, or carbocyclyl ring, wherein the spiro or fused six-membered heteroaryl, heterocyclyl, aryl, or carbocyclyl ring is optionally substituted H, F, Cl, Br, I, C1-C12 alkyl, or OH;

PML is a protease-cleavable, non-peptide linker attached to R2 or the fused heteroaryl or heterocyclyl formed by R1 and R2; and having the formula:

-Str-PM-Y—

where Str is a stretcher unit; PM is a peptidomimetic unit, and Y is a spacer unit; and

X is a reactive functional group selected from maleimide, thiol, amino, bromide, bromoacetamido, iodoacetamido, p-toluenesulfonate, iodide, hydroxyl, carboxyl, pyridyl disulfide, and N-hydroxysuccinimide.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIGS. 1A-1F: Intracellular stores of MRSA are protected from vancomycin in vivo and in vitro. FIG. 1A shows a schematic of the experimental design for generating free bacteria (planktonic) vs. intracellular bacteria. Four cohorts of mice were infected by intravenous injection with roughly equivalent doses of viable free bacteria or intracellular bacteria and selected groups were treated with vancomycin immediately after infection and then once per day (see Example 2). FIG. 1B and FIG. 1C show bacterial loads in kidney and brain, respectively of infected mice 4 days post infection. The dashed line indicates the limit of detection for the assay. FIG. 1D shows that MRSA is protected from vancomycin when cultured on a monolayer of infectable cells. (ND=none detected). FIG. 1E and FIG. 1F show that MRSA is able to grow in the presence of vancomyicn when cultured on a monolayer of infectable cells. MRSA (free bacteria) was seeded in media, media+vancomycin, or media+vancomycin and plated on a monolayer of MG63 osteoblasts (FIG. 1E) or Human Brain Microvascular Endothelial Cells (HBMEC, FIG. 1F). Extracellular bacteria (free bacteria) grew well in media alone, but were killed by vancomycin. In wells containing a monolayer of mammalian cells (Intracellular+vanco) a fraction of the bacteria were protected from vancomycin during the first 8 hours after infection and were able to expand within the intracellular compartment over 24 hours. Error bars show standard deviation for triplicate wells.

FIG. 2: shows the concept of an Antibody Antibiotic Conjugate (AAC). In one example, the AAC consists of an antibody directed against an epitope on the surface of S. aureus linked to a potent rifamycin-type antibiotic (e.g. Rifalog) via a linker that is cleaved by lysosomal proteases.

FIG. 3 shows a possible mechanism of drug activation for antibody-antibiotic conjugates (AAC). AACs bind to extracellular bacteria via the antigen binding domain (Fab) of the antibody and promote uptake of the opsonized bacteria via Fc-mediated phagocytosis. The linker is cleaved by lysosomal proteases such as cathepsin B. Following cleavage of the linker, the linker is hydrolyzed releasing free antibiotic inside the phagolysosome. The free antibiotic kills the opsonized and phagocytosed bacteria along with any previously internalized bacteria residing in the same compartment.

FIGS. 4A and 4B show aspects of serine-aspartate (SDR) proteins. FIG. 4A shows alignment of SDR proteins revealed by mass-spectrometry from S. aureus and S. epidermidis. SDR-regions are indicated by hatches. The rF1 epitope is expressed in abundance since there are multiple SDR proteins on S. aureus and multiple epitopes per protein. FIG. 4A discloses ‘SDSDSDSD’ as SEQ ID NO: 27. FIG. 4B is a model showing the step-wise glycosylation of SDR proteins by SdgA and SdgB. First, SdgB appends GlcNAc moieties onto the SD-region on SDR proteins, followed by additional GlcNAc modification by SdgA. The epitope for mAb rF1 includes the SdgB-dependent GlcNAc moieties. Data suggests that rF1 binds to GlcNac and parts of the SD backbone. FIG. 4B discloses ‘SDSDSD’ as SEQ ID NO: 28.

FIGS. 5A, 5B and 5C show mAb rF1 exhibits robust binding to and killing of S. aureus bacteria. (FIGS. 5A-C) Bacteria were preopsonized with huIgG1 mAbs rF1 (squares), 4675 anti-ClfA (triangles), or anti-herpes virus gD (circles). (FIG. 5A): Binding of mAbs to WT (USA300-Aspa) bacteria was assessed by flow cytometry, and expressed as mean fluorescent intensity (MFI). (FIG. 5B): CFSE-labeled, preopsonized WT (USA300-Aspa) bacteria were incubated with human PMN. Bacterial uptake was expressed as % of CFSE-positive PMN, after gating for CD11b-positive cells by flow cytometry. (FIG. 5C): Preopsonized WT (USA300-Aspa) bacteria were incubated with PMN to assess bacterial killing. Numbers of viable CFU per mL are representative of at least three experiments.

FIG. 6 shows flow cytometry analysis of binding of rF1 to S. aureus from various infected tissues. Homogenized tissues were double stained with mAb rF1 (X-axis), and with anti-peptidoglycan mAb 702 to distinguish bacteria from tissue debris (Y-axis) (left panel; gate indicated by arrow), followed by gating of bacteria to generate histogram figures (see also, Hazenbos et al. (2013) PLOS Pathogens 9 (10):1-18, FIG. 1D).

FIGS. 7A and 7B show binding of rF1 to various staphylococcal and non-staphylococcal Gram-positive bacterial species by flow cytometry (see also, Hazenbos et al. (2013) PLOS Pathogens 9 (10):1-18, FIG. 1E).

FIG. 8 shows selection of a potent rifamycin-type antibiotic (rifalog) dimethylpipBOR for its ability to kill non-replicating MRSA.

FIG. 9: Growth inhibition assay demonstrating that intact TAC (a form of AAC) does not kill planktonic bacteria unless the antibiotic is released by treatment with cathepsin B. TAC was incubated in buffer alone (open circles) or treated with cathepsin B (closed circles). The intact TAC was not able to prevent bacterial growth after overnight incubation. Pretreatment of the TAC with cathepsin B released sufficient antibiotic activity to prevent bacterial growth at 0.6 ug/mL of TAC, which is predicted to contain 0.006 ug/mL of antibiotic.

FIG. 10 shows efficacy of the rF1-AACs in an in vitro macrophage assay, as described in Example 19.

FIGS. 11A and 11B show the efficacy of the rF1-AACs in vivo as described in Example 20. Treatment of S. aureus infected mice with rF1-AACs greatly reduced or eradicated bacterial counts in infected organs as compared to naked antibody. FIG. 11A shows treatment with AAC containing 2 antibiotic molecules per antibody (AAR2) reduced bacterial load in the kidneys by approximately 30-fold and treatment with the AAC containing 4 antibiotic molecules per antibody (AAR4) reduced bacterial burdens by more than 30,000-fold. FIG. 11B shows that treatment with AAC AAR2 reduced bacterial burdens in the heart by approximately 70-fold with 6 out of 8 mice having undetectable level of bacteria in hearts; treatment with the AAC AAR4 completely eradicated infection in hearts resulting in 8 out of 8 mice having undetectable levels of bacteria.

### DETAILED DESCRIPTION OF EMBODIMENTS OF THE INVENTION

Reference will now be made in detail to certain embodiments of the invention, examples of which are illustrated in the accompanying structures and formulas. While the invention will be described in conjunction with the enumerated embodiments, including methods, materials and examples, such description is non-limiting and the invention is intended to cover all alternatives, modifications, and equivalents, whether they are generally known, or incorporated herein. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in the art to which this invention belongs. One skilled in the art will recognize many methods and materials similar or equivalent to those described herein, which could be used in the practice of the present invention. The present invention is in no way limited to the methods and materials described.

All publications, patent applications, patents, and other references mentioned herein are incorporated by reference in their entirety.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

The nomenclature used in this Application is based on IUPAC systematic nomenclature, unless indicated otherwise. Unless defined otherwise, technical and scientific terms used herein have the same meaning as commonly understood by one of ordinary skill in the art to which this invention belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

### II. Definitions

Staphylococcus aureus is also referred to herein as Staph A or S. aureus in short. Likewise, Staphylococcus epidermidis is also referred herein as Staph E or S. epidermidis.

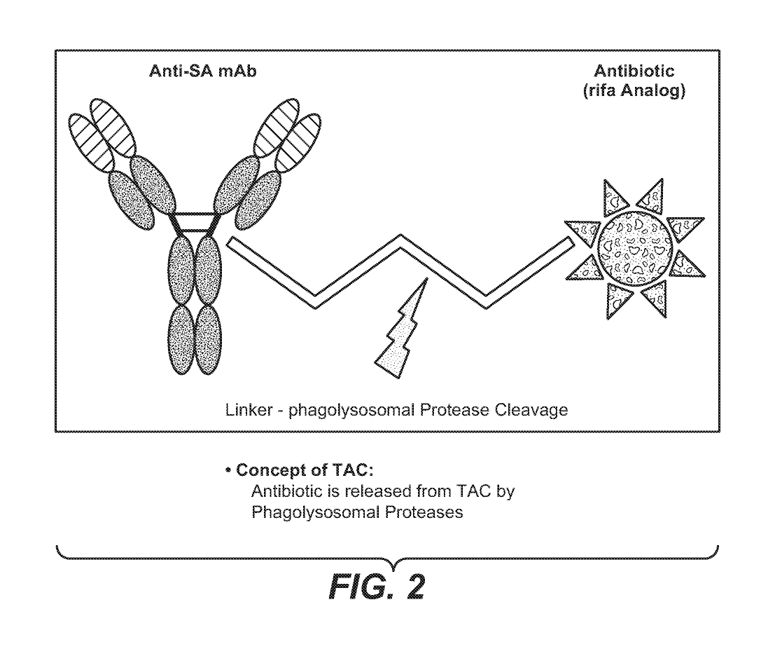

“Antibody Antibiotic Conjugate” or AAC is a compound composed of an antibody that is chemically linked to an antibiotic by a linker. The antibody binds an antigen or epitope on a bacterial surface, for example, a bacterial cell wall component. As used in this invention, the linker is a protease-cleavable, non-peptide linker that is designed to be cleaved by proteases, including cathepsin B, a lysosomal protease found in most mammalian cell types (Dubowchik et al (2002) Bioconj. Chem. 13:855-869). A diagram of the AAC with its 3 components is depicted in FIG. 2. “THIOMAB™ Antibiotic Conjugate” or “TAC” is a form of AAC in which the antibody is chemically conjugated to a linker-antibiotic unit via one or more cysteines, generally a cysteine that is recombinantly engineered into the antibody at specific site(s) on the antibody to not interfere with the antigen binding function.

When indicating the number of substituents, the term “one or more” refers to the range from one substituent to the highest possible number of substitution, i.e. replacement of one hydrogen up to replacement of all hydrogens by substituents. The term “substituent” denotes an atom or a group of atoms replacing a hydrogen atom on the parent molecule. The term “substituted” denotes that a specified group bears one or more substituents. Where any group may carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

The term “antibiotic” (abx or Abx) includes any molecule that specifically inhibits the growth of or kill micro-organisms, such as bacteria, but is non-lethal to the host at the concentration and dosing interval administered. In a specific aspect, an antibiotic is non-toxic to the host at the administered concentration and dosing intervals. Antibiotics effective against bacteria can be broadly classified as either bactericidal (i.e., directly kills) or bacteriostatic (i.e., prevents division). Anti-bactericidal antibiotics can be further subclassified as narrow-spectrum or broad-spectrum. A broad-spectrum antibiotic is one effective against a broad range of bacteria including both Gram-positive and Gram-negative bacteria, in contrast to a narrow-spectrum antibiotic, which is effective against a smaller range or specific families of bacteria. Examples of antibiotics include: (i) aminoglycosides, e.g., amikacin, gentamicin, kanamycin, neomycin, netilmicin, streptomycin, tobramycin, paromycin, (ii) ansamycins, e.g., geldanamycin, herbimycin, (iii) carbacephems, e.g., loracarbef, (iv), carbapenems, e.g., ertapenum, doripenem, imipenem/cilastatin, meropenem, (v) cephalosporins (first generation), e.g., cefadroxil, cefazolin, cefalotin, cefalexin, (vi) cephalosporins (second generation), e.g., ceflaclor, cefamandole, cefoxitin, cefprozil, cefuroxime, (vi) cephalosporins (third generation), e.g., cefixime, cefdinir, cefditoren, cefoperazone, cefotaxime, cefpodoxime, ceftazidime, ceftibuten, ceftizoxime, ceftriaxone, (vii) cephalosporins (fourth generation), e.g., cefepime, (viii), cephalosporins (fifth generation), e.g., ceftobiprole, (ix) glycopeptides, e.g., teicoplanin, vancomycin, (x) macrolides, e.g., axithromycin, clarithromycin, dirithromycine, erythromycin, roxithromycin, troleandomycin, telithromycin, spectinomycin, (xi) monobactams, e.g., axtreonam, (xii) penicilins, e.g., amoxicillin, ampicillin, axlocillin, carbenicillin, cloxacillin, dicloxacillin, flucloxacillin, mezlocillin, meticillin, nafcilin, oxacillin, penicillin, peperacillin, ticarcillin, (xiii) antibiotic polypeptides, e.g., bacitracin, colistin, polymyxin B, (xiv) quinolones, e.g., ciprofloxacin, enoxacin, gatifloxacin, levofloxacin, lemefloxacin, moxifloxacin, norfloxacin, orfloxacin, trovafloxacin, (xv) sulfonamides, e.g., mafenide, prontosil, sulfacetamide, sulfamethizole, sulfanilamide, sulfasalazine, sulfisoxazole, trimethoprim, trimethoprim-sulfamethoxazole (TMP-SMX), (xvi) tetracyclines, e.g., demeclocycline, doxycycline, minocycline, oxytetracycline, tetracycline and (xvii) others such as arspenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin or tinidazole.

The term “methicillin-resistant Staphylococcus aureus” (MRSA), alternatively known as multidrug resistant Staphylococcus aureus or oxacillin-resistant Staphylococcus aureus (ORSA), refers to any strain of Staphylococcus aureus that is resistant to beta-lactam antibiotics, which in include the penicillins (e.g., methicillin, dicloxacillin, nafcillin, oxacillin, etc.) and the cephalosporins. “Methicillin-sensitive Staphylococcus aureus” (MSSA) refers to any strain of Staphylococcus aureus that is sensitive to beta-lactam antibiotics.

The term “minimum inhibitory concentration” (“MIC”) refers to the lowest concentration of an antimicrobial that will inhibit the visible growth of a microorganism after overnight incubation. Assay for determining MIC are known. One method is as described in the Example section below.

The terms “anti-Staph a antibody” and “an antibody that binds to Staph a” refer to an antibody that is capable of binding an antigen on Staphylococcus aureus (“S. aureus”) with sufficient affinity such that the antibody is useful as a diagnostic and/or therapeutic agent in targeting S. aureus. In one embodiment, the extent of binding of an anti-Staph a antibody to an unrelated, non-Staph a protein is less than about 10% of the binding of the antibody to MRSA as measured, e.g., by a radioimmunoassay (RIA). In certain embodiments, an antibody that binds to Staph a has a dissociation constant (Kd) of ≤1 μM, ≤100 nM, ≤10 nM, ≤5 Nm, ≤4 nM, ≤3 nM, ≤2 nM, ≤1 nM, ≤0.1 nM, ≤0.01 nM, or ≤0.001 nM (e.g., 10-8 M or less, e.g. from 10-8 M to 10-13 M, e.g., from 10-9 M to 10-13 M). In certain embodiments, an anti-Staph a antibody binds to an epitope of Staph a that is conserved among Staph from different species. An anti-Staph antibody herein will refer to an antibody that binds to at least one more Staphylococcal species in addition S. Aureus.

“SDR” refers to serine-aspartate repeat; SDRs are present in a family of cell wall proteins, characterized by a large stretch of serine-aspartate dipeptide repeats adjacent to an adhesive A-domain, that is present in staphylococci (Foster T J, Hook M (1998) Trends Microbiol 6: 484-488). Such proteins involved in adherence include clumping factor (Clf)A and ClfB. In addition to ClfA and ClfB, S. aureus also expresses three SDR-proteins, SdrC, SdrD and SdrE, Three additional members of this family, SrdF, SdrG and SdrH, are present in most S. epidermidis strains (McCrea K W, et al. (2000) The serine-aspartate repeat (Sdr) protein family in Staphylococcus epidermidis. Microbiology 146 (Pt 7): 1535-1546). In each of these proteins, the SDR-region, which contains between 25 and 275 SD-dipeptide repeats (SEQ ID NO: 24), is located between the N-terminal ligand-binding A-domain and a C-terminal LPXTG-motif (SEQ ID NO: 25),

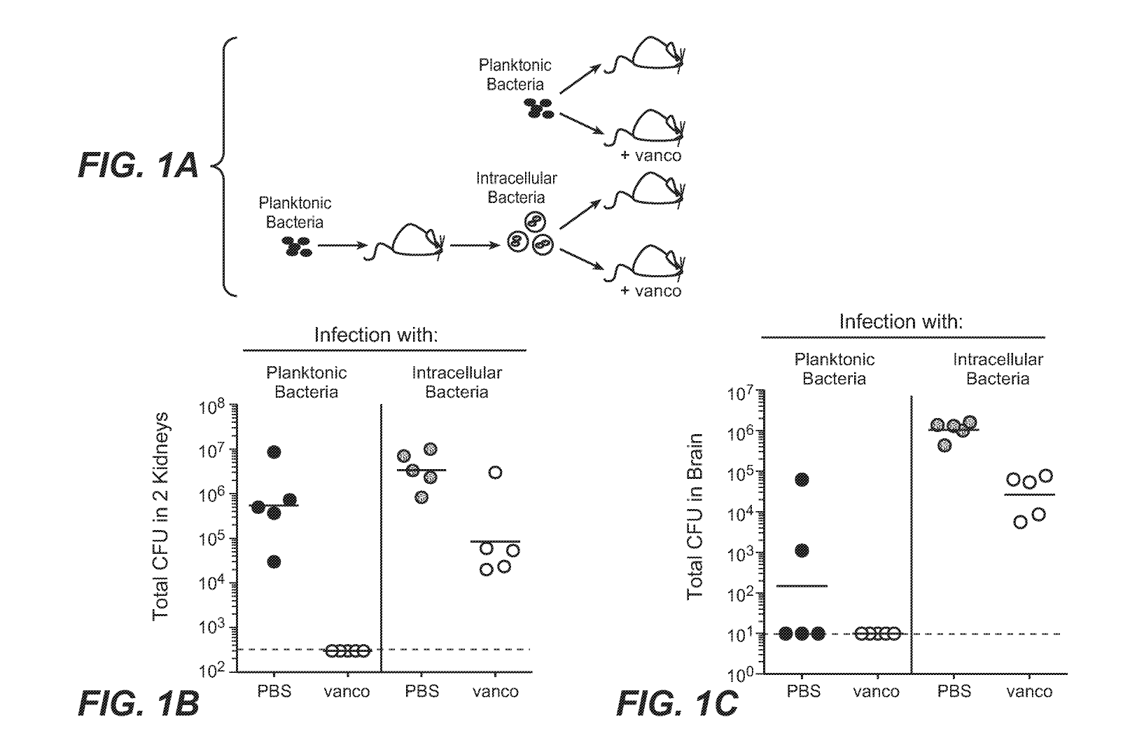

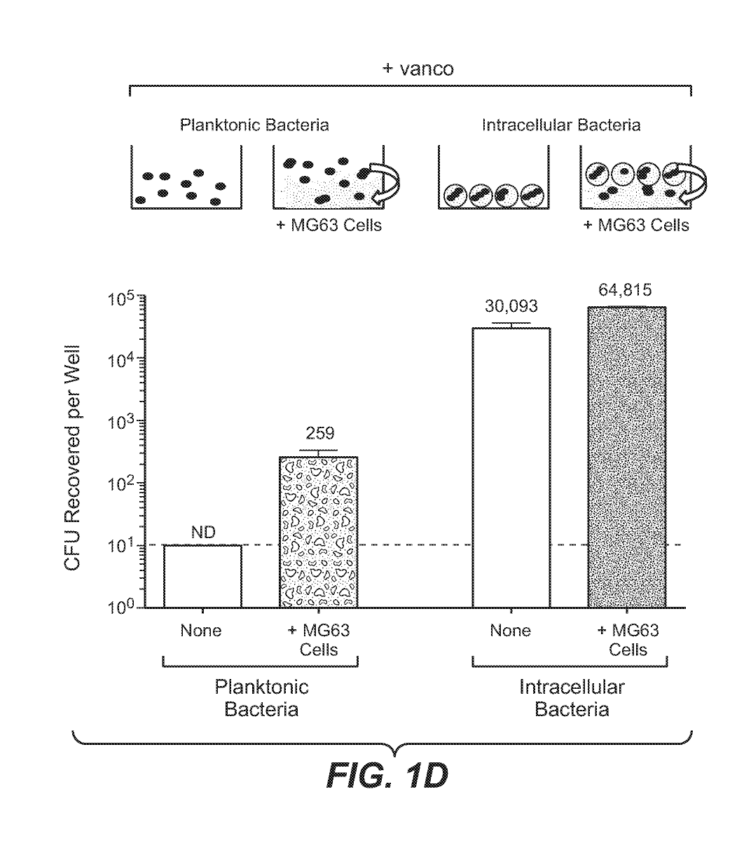

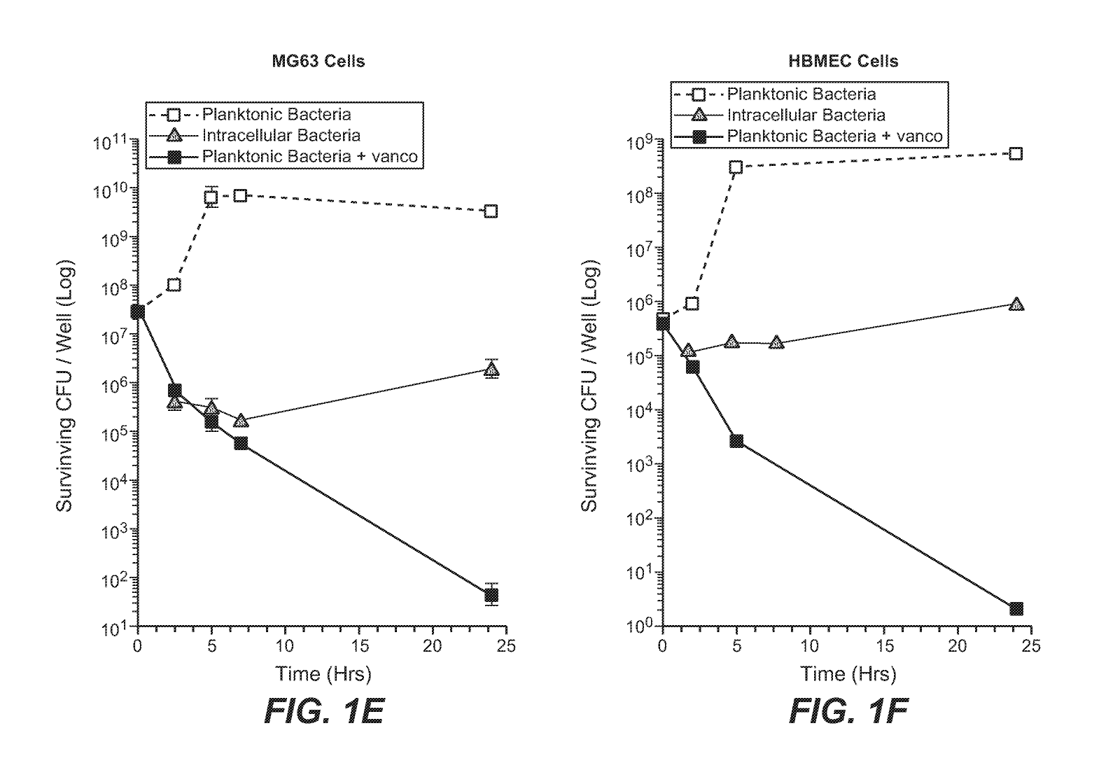

The antibody designated “F1” has heavy chain and light chain variable domain sequences as depicted in FIG. 1 of U.S. Pat. No. 8,617,556, which is incorporated herein by reference in its entirety. The CDR sequences of F1, which in particular contribute to the antigen-binding properties of F1, are also depicted in FIG. 1. Antibody F1 is fully human, is capable of specifically binding Staphylococcus species such as S. aureus and S. epidermidis. Importantly, antibody F1 is capable of binding whole bacteria in vivo as well as in vitro. Furthermore, antibody F1 is capable of binding to bacteria that have been grown in infected tissue of, for example, an animal. Recombinantly produced F1 is herein also called “rF1”. rF1 (and F1) antibody is an anti-SDR monoclonal Ab. The epitope for mAb rF1 includes the SdgB-dependent GlcNAc moieties. Data suggests that rF1 binds to GlcNac and parts of the SD backbone. “rF1 antibody” as used herein encompasses the F1 antibody, the rF1 antibody as well as all variants of rF1 containing amino acid alterations relative to rF1. The amino acid sequences of the rF1 and variant antibodies are provided below.

The term “antibody” herein is used in the broadest sense and specifically covers monoclonal antibodies, polyclonal antibodies, dimers, multimers, multispecific antibodies (e.g., bispecific antibodies), and antigen binding antibody fragments thereof, (Miller et al (2003) J. of Immunology 170:4854-4861). Antibodies may be murine, human, humanized, chimeric, or derived from other species. An antibody is a protein generated by the immune system that is capable of recognizing and binding to a specific antigen (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may be recognized and bound by more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease, an infected cell or a microorganism such as a bacterium. The immunoglobulin (Ig) disclosed herein can be of any isotype except IgM (e.g., IgG, IgE, IgD, and IgA) and subclass (e.g., IgG1, IgG2, IgG3, IgG4, IgA1 and IgA2. The immunoglobulins can be derived from any species. In one aspect, the Ig is of human, murine, or rabbit origin. In a specific embodiment, the Ig is of human origin.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 6, c, y, and t, respectively.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The terms “full length antibody,” “intact antibody,” and “whole antibody” are used herein interchangeably to refer to an antibody having a structure substantially similar to a native antibody structure or having heavy chains that contain an Fc region as defined herein.

An “antigen-binding fragment” of an antibody refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation (e.g., natural variation in glycosylation), such variants generally being present in minor amounts. One such possible variant for IgG1 antibodies is the cleavage of the C-terminal lysine (K) of the heavy chain constant region. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In addition to their specificity, the monoclonal antibodies are advantageous in that they may be synthesized uncontaminated by other antibodies.

The term “chimeric antibody” refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized antibody” refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region,” “HVR,” or “HV,” when used herein refers to the regions of an antibody variable domain which are hypervariable in sequence (“complementarity determining regions” or “CDRs”) and/or form structurally defined loops and/or contain the antigen-contacting residues (“antigen contacts”). Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain (Hamers-Casterman et al., (1993) Nature 363:446-448; Sheriff et al., (1996) Nature Struct. Biol. 3:733-736).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, (1987) J. Mol. Biol. 196:901-917). For antigen contacts, refer to MacCallum et al. J. Mol. Biol. 262: 732-745 (1996). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. Unless otherwise indicated, HVR residues, CDR residues and other residues in the variable domain (e.g., FR residues) are numbered herein according to Kabat et al., supra.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

The term “Fc region” herein is used to define a C-terminal region of an immunoglobulin heavy chain. The term includes native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system—also called the EU index, as described in Kabat et al., Sequences of Proteins of Immunological Interest, 5th Ed. Public Health Service, National Institutes of Health, Bethesda, Md., 1991) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279(8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “epitope” refers to the particular site on an antigen molecule to which an antibody binds.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

A “naked antibody” refers to an antibody that is not conjugated to a heterologous moiety (e.g., a cytotoxic moiety) or radiolabel. The naked antibody may be present in a pharmaceutical formulation.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express Fc□□gamma)RIII only, whereas monocytes express Fc□□gamma)RI, Fc□□gamma)RII and Fc□□gamma)RIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Phagocytosis” refers to a process by which a pathogen is engulfed or internalized by a host cell (e.g., macrophage or neutrophil). Phagocytes mediate phagocytosis by three pathways: (i) direct cell surface receptors (for example, lectins, integrins and scavenger receptors) (ii) complement enhanced—using complement receptors (including CRI, receptor for C3b, CR3 and CR4) to bind and ingest complement opsonized pathogens, and (iii) antibody enhanced—using Fc Receptors (including Fc gammaRI, Fc□gammaRIIA and Fc□gammaRIIIA) to bind antibody opsonized particles which then become internalized and fuse with lysosomes to become phagolysosomes. In the present invention, it is believed that pathway (iii) plays a significant role in the delivery of the anti-MRSA AAC therapeutics to infected leukocytes, e.g., neutrophils and macrophages (Phagocytosis of Microbes: complexity in Action by D. Underhill and A Ozinsky. (2002) Annual Review of Immunology, Vol 20:825).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed.

The carbohydrate attached to the Fc region may be altered. Native antibodies produced by mammalian cells typically comprise a branched, biantennary oligosaccharide that is generally attached by an N-linkage to Asn297 of the CH2 domain of the Fc region. See, e.g., Wright et al. (1997) TIBTECH 15:26-32. The oligosaccharide may include various carbohydrates, e.g., mannose, N-acetyl glucosamine (GIcNAc), galactose, and sialic acid, as well as a fucose attached to a GIcNAc in the “stem” of the biantennary oligosaccharide structure. In some embodiments, modifications of the oligosaccharide in an IgG may be made in order to create IgGs with certain additionally improved properties. For example, antibody modifications are provided having a carbohydrate structure that lacks fucose attached (directly or indirectly) to an Fc region. Such modifications may have improved ADCC function. See, e.g. US 2003/0157108 (Presta, L.); US 2004/0093621 (Kyowa Hakko Kogyo Co., Ltd). Examples of publications related to “defucosylated” or “fucose-deficient” antibody modifications include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336: 1239-1249 (2004); Yamane-Ohnuki et al. Biotech. Bioeng. 87: 614 (2004). Examples of cell lines capable of producing defucosylated antibodies include Lee 13 CHO cells deficient in protein fucosylation (Ripka et al. Arch. Biochem. Biophys. 249:533-545 (1986); US Pat. Appl. Pub. No. 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al, Biotechnol. Bioeng., 94(4):680-688 (2006); and WO2003/085107).

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a rF1 antibody” refers to one or more nucleic acid molecules encoding antibody heavy and light chains, including such nucleic acid molecule(s) in a single vector or separate vectors, and such nucleic acid molecule(s) present at one or more locations in a host cell.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to a target unrelated to rF1 is less than about 10% of the binding of the antibody to the target as measured, e.g., by a radioimmunoassay (RIA). In certain embodiments, an antibody that specifically binds to rF1 has a dissociation constant (Kd) of ≤1 μM, ≤100 nM, ≤10 nM, ≤1 nM, or ≤0.1 nM. In certain embodiments, an antibody specifically binds to an epitope on that is conserved from different species. In another embodiment, specific binding can include, but does not require exclusive binding.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., (1999) J. Mol. Biol. 293:865-881). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20TM surfactant in PBS. When the plates have dried, 150 l/well of scintillant (MICROSCINT-20TM; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd is measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20TM surfactant (PBST) at 25° C. at a flow rate of approximately 25 μl/min. Association rates (kon) and dissociation rates (koff) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M−1 s−1 by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kon” according to this invention can also be determined as described above using a BIACORE®-2000 or a BIACORE®-3000 system (BIAcore, Inc., Piscataway, N.J.).

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described.

The term “rifamycin-type antibiotic” means the class or group of antibiotics having the structure of, or similar structure to, rifamycin.

The term “rifalazil-type antibiotic” means the class or group of antibiotics having the structure of, or similar structure to, rifalazil.

When indicating the number of substituents, the term “one or more” refers to the range from one substituent to the highest possible number of substitution, i.e. replacement of one hydrogen up to replacement of all hydrogens by substituents. The term “substituent” denotes an atom or a group of atoms replacing a hydrogen atom on the parent molecule. The term “substituted” denotes that a specified group bears one or more substituents. Where any group may carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

The term “alkyl” as used herein refers to a saturated linear or branched-chain monovalent hydrocarbon radical of one to twelve carbon atoms (C1-C12), wherein the alkyl radical may be optionally substituted independently with one or more substituents described below. In another embodiment, an alkyl radical is one to eight carbon atoms (C1-C8), or one to six carbon atoms (C1-C6). Examples of alkyl groups include, but are not limited to, methyl (Me, —CH3), ethyl (Et, —CH2CH3), 1-propyl (n-Pr, n-propyl, —CH2CH2CH3), 2-propyl (i-Pr, i-propyl, —CH(CH3)2), 1-butyl (n-Bu, n-butyl, —CH2CH2CH2CH3), 2-methyl-1-propyl (i-Bu, i-butyl, —CH2CH(CH3)2), 2-butyl (s-Bu, s-butyl, —CH(CH3)CH2CH3), 2-methyl-2-propyl (t-Bu, t-butyl, —C(CH3)3), 1-pentyl (n-pentyl, —CH2CH2CH2CH2CH3), 2-pentyl (—CH(CH3)CH2CH2CH3), 3-pentyl (—CH(CH2CH3)2), 2-methyl-2-butyl (—C(CH3)2CH2CH3), 3-methyl-2-butyl (—CH(CH3)CH(CH3)2), 3-methyl-1-butyl (—CH2CH2CH(CH3)2), 2-methyl-1-butyl (—CH2CH(CH3)CH2CH3), 1-hexyl (—CH2CH2CH2CH2CH2CH3), 2-hexyl (—CH(CH3)CH2CH2CH2CH3), 3-hexyl (—CH(CH2CH3)(CH2CH2CH3)), 2-methyl-2-pentyl (—C(CH3)2CH2CH2CH3), 3-methyl-2-pentyl (—CH(CH3)CH(CH3)CH2CH3), 4-methyl-2-pentyl (—CH(CH3)CH2CH(CH3)2), 3-methyl-3-pentyl (—C(CH3)(CH2CH3)2), 2-methyl-3-pentyl (—CH(CH2CH3)CH(CH3)2), 2,3-dimethyl-2-butyl (—C(CH3)2CH(CH3)2), 3,3-dimethyl-2-butyl (—CH(CH3)C(CH3)3, 1-heptyl, 1-octyl, and the like.

The term “alkylene” as used herein refers to a saturated linear or branched-chain divalent hydrocarbon radical of one to twelve carbon atoms (C1-C12), wherein the alkylene radical may be optionally substituted independently with one or more substituents described below. In another embodiment, an alkylene radical is one to eight carbon atoms (C1-C8), or one to six carbon atoms (C1-C6). Examples of alkylene groups include, but are not limited to, methylene (—CH2-), ethylene (—CH2CH2-), propylene (—CH2CH2CH2-), and the like.

The term “alkenyl” refers to linear or branched-chain monovalent hydrocarbon radical of two to eight carbon atoms (C2-C8) with at least one site of unsaturation, i.e., a carbon-carbon, sp2 double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH2), allyl (—CH2CH═CH2), and the like.

The term “alkenylene” refers to linear or branched-chain divalent hydrocarbon radical of two to eight carbon atoms (C2-C8) with at least one site of unsaturation, i.e., a carbon-carbon, sp2 double bond, wherein the alkenylene radical may be optionally substituted independently with one or more substituents described herein, and includes radicals having “cis” and “trans” orientations, or alternatively, “E” and “Z” orientations. Examples include, but are not limited to, ethylenylene or vinylene (—CH═CH—), allyl (—CH2CH═CH—), and the like.

The term “alkynyl” refers to a linear or branched monovalent hydrocarbon radical of two to eight carbon atoms (C2-C8) with at least one site of unsaturation, i.e., a carbon-carbon, sp triple bond, wherein the alkynyl radical may be optionally substituted independently with one or more substituents described herein. Examples include, but are not limited to, ethynyl (—C≡CH), propynyl (propargyl, —CH2C≡CH), and the like.

The term “alkynylene” refers to a linear or branched divalent hydrocarbon radical of two to eight carbon atoms (C2-C8) with at least one site of unsaturation, i.e., a carbon-carbon, sp triple bond, wherein the alkynylene radical may be optionally substituted independently with one or more substituents described herein. Examples include, but are not limited to, ethynylene (—C≡C—), propynylene (propargylene, —CH2C≡C—), and the like.

The terms “carbocycle”, “carbocyclyl”, “carbocyclic ring” and “cycloalkyl” refer to a monovalent non-aromatic, saturated or partially unsaturated ring having 3 to 12 carbon atoms (C3-C12) as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, and bicyclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane. Spiro moieties are also included within the scope of this definition. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like. Carbocyclyl groups are optionally substituted independently with one or more substituents described herein.

“Aryl” means a monovalent aromatic hydrocarbon radical of 6-20 carbon atoms (C6-C20) derived by the removal of one hydrogen atom from a single carbon atom of a parent aromatic ring system. Some aryl groups are represented in the exemplary structures as “Ar”. Aryl includes bicyclic radicals comprising an aromatic ring fused to a saturated, partially unsaturated ring, or aromatic carbocyclic ring. Typical aryl groups include, but are not limited to, radicals derived from benzene (phenyl), substituted benzenes, naphthalene, anthracene, biphenyl, indenyl, indanyl, 1,2-dihydronaphthalene, 1,2,3,4-tetrahydronaphthyl, and the like. Aryl groups are optionally substituted independently with one or more substituents described herein.

“Arylene” means a divalent aromatic hydrocarbon radical of 6-20 carbon atoms (C6-C20) derived by the removal of two hydrogen atom from a two carbon atoms of a parent aromatic ring system. Some arylene groups are represented in the exemplary structures as “Ar”. Arylene includes bicyclic radicals comprising an aromatic ring fused to a saturated, partially unsaturated ring, or aromatic carbocyclic ring. Typical arylene groups include, but are not limited to, radicals derived from benzene (phenylene), substituted benzenes, naphthalene, anthracene, biphenylene, indenylene, indanylene, 1,2-dihydronaphthalene, 1,2,3,4-tetrahydronaphthyl, and the like. Arylene groups are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

A “metabolite” is a product produced through metabolism in the body of a specified compound or salt thereof. Metabolites of a compound may be identified using routine techniques known in the art and their activities determined using tests such as those described herein. Such products may result for example from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a Formula I compound of this invention with a mammal for a period of time sufficient to yield a metabolic product thereof.

The term “pharmaceutical formulation” refers to a preparation which is in such form as to permit the biological activity of an active ingredient contained therein to be effective, and which contains no additional components which are unacceptably toxic to a subject to which the formulation would be administered.

A “sterile” formulation is aseptic or free from all living microorganisms and their spores.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present invention are hypertonic as a result of the addition of salt and/or buffer.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS™.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. A “pharmaceutically acceptable acid” includes inorganic and organic acids which are nontoxic at the concentration and manner in which they are formulated. For example, suitable inorganic acids include hydrochloric, perchloric, hydrobromic, hydroiodic, nitric, sulfuric, sulfonic, sulfinic, sulfanilic, phosphoric, carbonic, etc. Suitable organic acids include straight and branched-chain alkyl, aromatic, cyclic, cycloaliphatic, arylaliphatic, heterocyclic, saturated, unsaturated, mono, di- and tri-carboxylic, including for example, formic, acetic, 2-hydroxyacetic, trifluoroacetic, phenylacetic, trimethylacetic, t-butyl acetic, anthranilic, propanoic, 2-hydroxypropanoic, 2-oxopropanoic, propandioic, cyclopentanepropionic, cyclopentane propionic, 3-phenylpropionic, butanoic, butandioic, benzoic, 3-(4-hydroxybenzoyl)benzoic, 2-acetoxy-benzoic, ascorbic, cinnamic, lauryl sulfuric, stearic, muconic, mandelic, succinic, embonic, fumaric, malic, maleic, hydroxymaleic, malonic, lactic, citric, tartaric, glycolic, glyconic, gluconic, pyruvic, glyoxalic, oxalic, mesylic, succinic, salicylic, phthalic, palmoic, palmeic, thiocyanic, methanesulphonic, ethanesulphonic, 1,2-ethanedisulfonic, 2-hydroxyethanesulfonic, benzenesulphonic, 4-chorobenzenesulfonic, napthalene-2-sulphonic, p-toluenesulphonic, camphorsulphonic, 4-methylbicyclo[2.2.2]-oct-2-ene-1-carboxylic, glucoheptonic, 4,4′-methylenebis-3-(hydroxy-2-ene-1-carboxylic acid), hydroxynapthoic.

“Pharmaceutically-acceptable bases” include inorganic and organic bases which are non-toxic at the concentration and manner in which they are formulated. For example, suitable bases include those formed from inorganic base forming metals such as lithium, sodium, potassium, magnesium, calcium, ammonium, iron, zinc, copper, manganese, aluminum, N-methylglucamine, morpholine, piperidine and organic nontoxic bases including, primary, secondary and tertiary amines, substituted amines, cyclic amines and basic ion exchange resins, [e.g., N(R′)4+(where R′ is independently H or C1-4 alkyl, e.g., ammonium, Tris)], for example, isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional pharmaceutically acceptable acids and bases useable with the present invention include those which are derived from the amino acids, for example, histidine, glycine, phenylalanine, aspartic acid, glutamic acid, lysine and asparagine.

“Pharmaceutically acceptable” buffers and salts include those derived from both acid and base addition salts of the above indicated acids and bases. Specific buffers and/or salts include histidine, succinate and acetate.

A “pharmaceutically acceptable sugar” is a molecule which, when combined with a protein of interest, significantly prevents or reduces chemical and/or physical instability of the protein upon storage. When the formulation is intended to be lyophilized and then reconstituted, “pharmaceutically acceptable sugars” may also be known as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

An “individual” or “subject” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” (and grammatical variations thereof such as “treat” or “treating”) refers to clinical intervention designed to alter the natural course of the individual, tissue or cell being treated during the course of clinical pathology. Desirable effects of treatment include, but are not limited to, decreasing the rate of disease progression, ameliorating or palliating the disease state, and remission or improved prognosis, all measurable by one of skill in the art such as a physician. In one embodiment, treatment can mean alleviation of symptoms, diminishment of any direct or indirect pathological consequences of the disease, decreasing the rate of infectious disease progression, amelioration or palliation of the disease state, and remission or improved prognosis. In some embodiments, the AACs and TACs of the invention are used to delay development of a disease or to slow the progression of an infectious disease or reduce the bacterial load in the blood stream and/or in infected tissues and organs.

As used herein, “in conjunction with” refers to administration of one treatment modality in addition to another treatment modality. As such, “in conjunction with” refers to administration of one treatment modality before, during or after administration of the other treatment modality to the individual.

The term “bacteremia” refers to the presence of bacteria in the bloodstream which is most commonly detected through a blood culture. Bacteria can enter the bloodstream as a severe complication of infections (like pneumonia or meningitis), during surgery (especially when involving mucous membranes such as the gastrointestinal tract), or due to catheters and other foreign bodies entering the arteries or veins. Bacteremia can have several consequences. The immune response to the bacteria can cause sepsis and septic shock, which has a relatively high mortality rate. Bacteria can also use the blood to spread to other parts of the body, causing infections away from the original site of infection. Examples include endocarditis or osteomyelitis.

A “therapeutically effective amount” is the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects. In one embodiment, a therapeutically effective amount is an amount effective to reduce bacteremia in an in vivo infection. In one aspect, a “therapeutically effective amount” is at least the amount effective to reduce the bacterial load or colony forming units (CFU) isolated from a patient sample such as blood by at least one log relative to prior to drug administration. In a more specific aspect, the reduction is at least 2 logs. In another aspect, the reduction is at least 3, 4, 5 logs. In yet another aspect, the reduction is to below detectable levels using assays known in the art including assays exemplified herein. In another embodiment, a therapeutically effective amount is the amount of an AAC in one or more doses given over the course of the treatment period, that achieves a negative blood culture (i.e., does not grow out the bacteria that is the target of the AAC) as compared to the positive blood culture before or at the start of treatment of the infected patient.

A “prophylactically effective amount” refers to an amount effective, at the dosages and for periods of time necessary, to achieve the desired prophylactic result. Typically but not necessarily, since a prophylactic dose is used in subjects prior to, at the earlier stage of disease, or even prior to exposure to conditions where the risk of infection is elevated, the prophylactically effective amount can be less than the therapeutically effective amount. In one embodiment, a prophylactically effective amount is at least an amount effective to reduce, prevent the occurrence of or spread of infection from one cell to another.

“Chronic” administration refers to administration of the medicament(s) in a continuous as opposed to acute mode, so as to maintain the initial therapeutic effect (activity) for an extended period of time. “Intermittent” administration is treatment that is not consecutively done without interruption, but rather is cyclic in nature.

The term “package insert” is used to refer to instructions customarily included in commercial packages of therapeutic products, that contain information about the indications, usage, dosage, administration, combination therapy, contraindications and/or warnings concerning the use of such therapeutic products.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “protecting group” refers to a substituent that is commonly employed to block or protect a particular functionality while other functional groups react on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

The term “about” as used herein refers to the usual error range for the respective value readily known to the skilled person in this technical field. Reference to “about” a value or parameter herein includes (ad describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

### III. Compositions and Methods

### Antibody-Antibiotic Conjugates (AAC)

The experimental results herein are a strong indication that therapies aimed at eliminating intracellular bacteria will improve clinical success. Towards this aim, the present invention provides a unique therapeutic that selectively kills S. aureus organisms that have invaded intracellular compartments of host cells. The present invention demonstrates that such a therapeutic is efficacious in in-vivo models where conventional antibiotics like vancomycin fail.

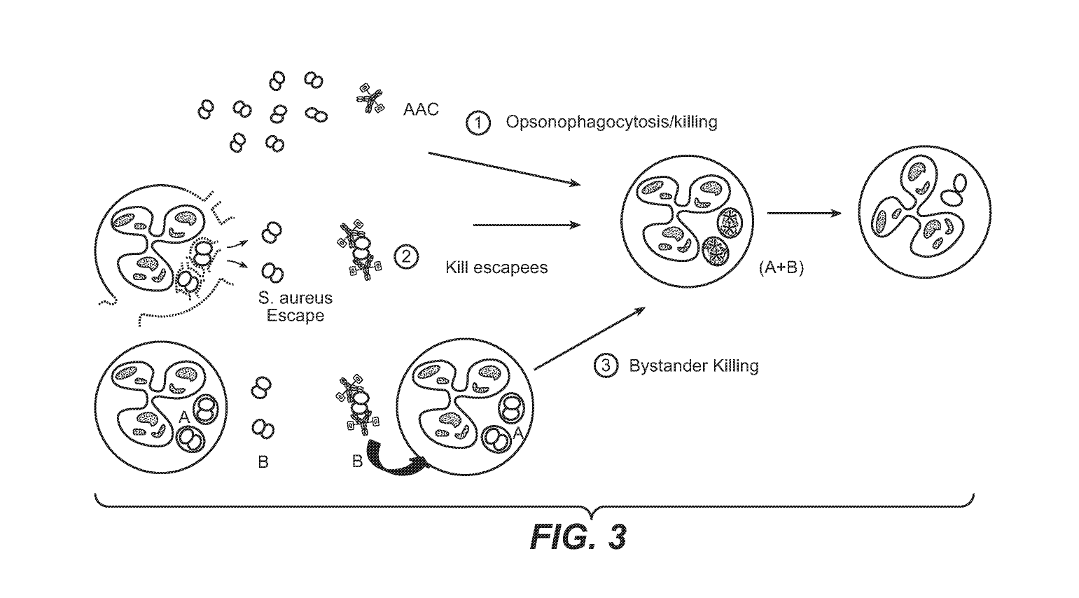

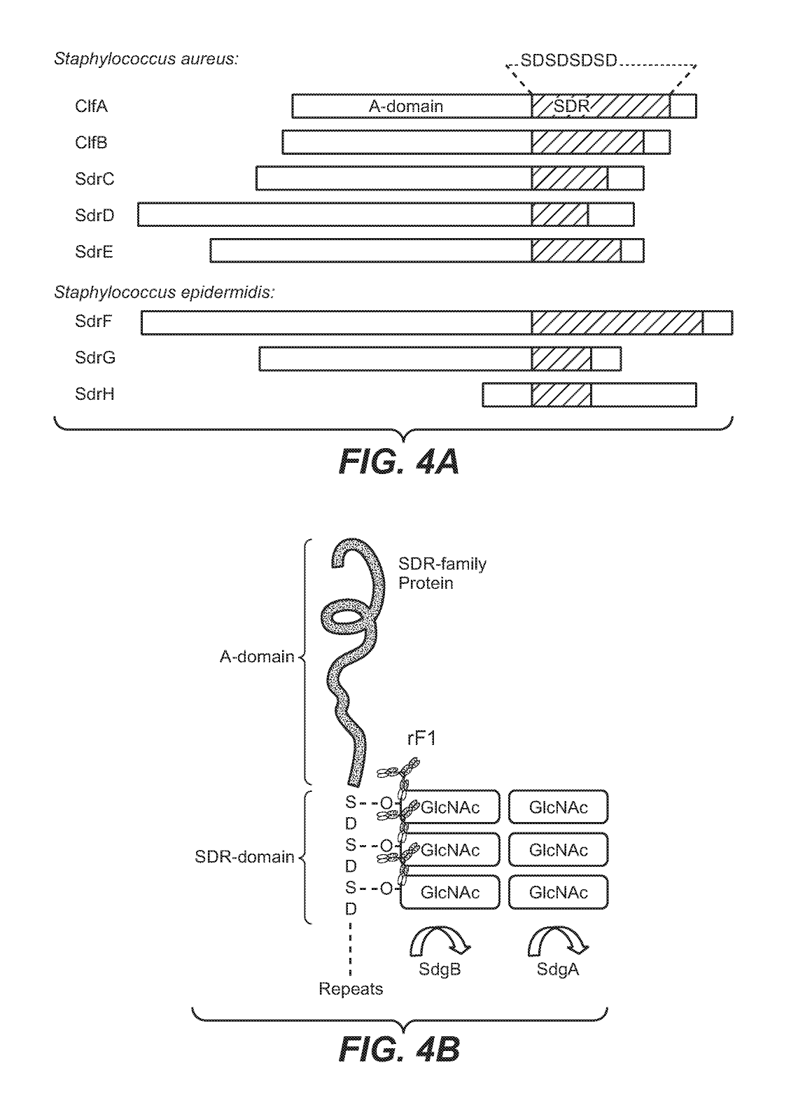

The invention provides an antibacterial therapy that aims to prevent antibiotic escape by targeting populations of bacteria that evade conventional antibiotic therapy. The novel antibacterial therapy is achieved with an Antibody Antibiotic Conjugate (AAC) in which an rF1 antibody specific for cell wall components found on S. aureus (including MRSA) is chemically linked to a potent rifamycin-type antibiotic (a derivative of rifamycin). The rifamycin-type antibiotic is joined to the antibody via a protease-cleavable, non-peptide linker that is designed to be cleaved by proteases, including cathepsin B, a lysosomal protease found in most mammalian cell types (Dubowchik et al (2002) Bioconj. Chem. 13:855-869). A diagram of the AAC with its 3 components is depicted in FIG. 2. Not to be limited by any one theory, one mechanism of action of the AAC is schematized in FIG. 3. The AAC acts as a pro-drug in that the rifamycin-type antibiotic is inactive (due to the large size of the antibody) until the linker is cleaved. Since a significant proportion of S. aureus found in a natural infection is taken up by host cells, primarily neutrophils and macrophages, at some point during the course of infection in the host, the time spent inside host cells provides a significant opportunity for the bacterium to evade antibiotic activity. The AACs of the invention are designed to bind to the Staph bacteria and release the antibiotic inside the phagolysosome after bacteria are taken up by host cells. By this mechanism, AAC are able to concentrate the active antibiotic specifically in a location where S. aureus is poorly treated by conventional antibiotics. While the invention is not limited or defined by an particular mechanism of action, the AAC improve antibiotic activity via three potential mechanisms: (1) The AAC delivers antibiotic inside mammalian cells that take up the bacteria, thereby increasing the potency of antibiotics that diffuse poorly into the phagolysosomes where bacteria are sequestered. (2) AAC opsonize bacteria thereby increasing uptake of free bacteria by phagocytic cells, and release the antibiotic locally to kill the bacteria while they are sequestered in the phagolysosome. Since thousands of AACs can bind to a single bacterium, this platform releases sufficient antibiotics in these intracellular niches to sustain maximal antimicrobial killing. Furthermore, as more bacteria are released from pre-existing intracellular reservoirs, the fast on-rate of this antibody-based therapy ensures immediate “tagging” of these bacteria before they can escape to neighboring or distant cells, thus mitigating further spread of the infection. (3) AAC improve the half-life of antibiotics in vivo (improved pharmacokinetics) by linking the antibiotic to an antibody, as compared to antibiotics which are cleared rapidly from serum. Improved pharmacokinetics of AAC enable delivery of sufficient antibiotic in regions where S. aureus is concentrated while limiting the overall dose of antibiotic that needs to be administered systemically. This property should permit long-term therapy with AAC to target persistent infection with minimal antibiotic side effects.

An antibody-antibiotic conjugate compound of the invention comprises an anti-SDR antibody covalently attached by a protease-cleavable, non-peptide linker via a recombinantly introduced cysteine, to a rifamycin-type antibiotic.

In an exemplary embodiment, the anti-SDR antibody (e.g. rF1 antibody) is a cysteine-engineered antibody comprising a recombinantly introduced cysteine amino acid.

In an exemplary embodiment, the protease-cleavable, non-peptide linker is covalently attached via a recombinantly introduced cysteine on the rF1, anti-SDR antibody, to the rifamycin-type antibiotic

An exemplary embodiment is the antibody-antibiotic conjugate having the formula:

Ab-(PM L-abx)ₚ

wherein:

Ab is the rF1 antibody;

PML is the protease-cleavable, non-peptide linker having the formula:

Str-PM-Y—

where Str is a stretcher unit; PM is a peptidomimetic unit, and Y is a spacer unit;

abx is the rifamycin-type antibiotic; and

p is an integer from 1 to 8.

The rifamycin-type antibiotic may be a rifalazil-type antibiotic.

The rifamycin-type antibiotic may comprise a quaternary amine attached to the protease-cleavable, non-peptide linker.

An exemplary embodiment of the antibody-antibiotic conjugate has Formula I:

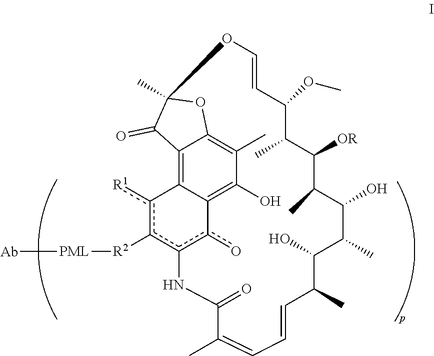

wherein:

the dashed lines indicate an optional bond;

R is H, C1-C12 alkyl, or C(O)CH3;

R¹ is OH;

R² is CH═N-(heterocyclyl), wherein the heterocyclyl is optionally substituted with one or more groups independently selected from C(O)CH₃, C₁-C₁₂ alkyl, C₁-C₁₂ heteroaryl, C₂-C₂₀ heterocyclyl, C₆-C₂₀ aryl, and C₃-C₁₂ carbocyclyl;

or R1 and R2 form a five- or six-membered fused heteroaryl or heterocyclyl, and optionally forming a spiro or fused six-membered heteroaryl, heterocyclyl, aryl, or carbocyclyl ring, wherein the spiro or fused six-membered heteroaryl, heterocyclyl, aryl, or carbocyclyl ring is optionally substituted H, F, Cl, Br, I, C1-C12 alkyl, or OH;

PML is the protease-cleavable, non-peptide linker attached to R2 or the fused heteroaryl or heterocyclyl formed by R1 and R2; and

Ab is the rF1 antibody.

The number of antibiotic moieties which may be conjugated via a reactive linker moiety to an antibody molecule may be limited by the number of free cysteine residues, which are introduced by the methods described herein. Exemplary AAC comprise antibodies which have 1, 2, 3, or 4 engineered cysteine amino acids (Lyon, R. et al (2012) Methods in Enzym. 502:123-138).

To be effective target on MRSA, the epitope is preferably highly abundant, stably expressed during infection and highly conserved in all clinical MRSA strains. The rF1 antibody fulfills these requirements and additionally, also binds to Staph epidermidis as well.

Anti-SDR and rF1 Antibodies

Anti-SDR antibodies can be produced as described below for the generation of F1 antibody. Several examples of anti-SDR antibodies are provided herein including rF1, SD2, SD3 and SD4.

The rF1 Abs will be described in detail here.

rF1 antibody is a fully human is capable of specifically binding Staphylococcus species such as S. aureus and S. epidermidis. Importantly, rF1 is capable of binding whole bacteria in vivo as well as in vitro. Furthermore, antibody rF1 is capable of binding to bacteria that have been grown in infected tissue of, for example, an animal. The rF1 Abs provided herein or functional equivalents thereof are capable of binding to S. aureus surface proteins ClfA, ClfB, SdrC, SdrD and SdrE.

Table 4A and Table 4B show an alignment of the H chain and L chain CDR sequences of the parent antibody F1, rF1 antibody and its variants. F1 and rF1 differ in sequence in FW1 and LC CDR3 (QHYXRFPYT, where X can be I or M (SEQ ID NO: 26); F1 is I (SEQ ID NO: 6) and rF1 is M (SEQ ID NO: 7)).

TABLE 4A
Heavy chain CDR sequences

| Anti- | | | |
|---|---|---|---|
| body | HC CDR1 | HC CDR2 | HC CDR3 |
| F1 | RFAMS | SINNGNNP | DHPSSGW |
| | (SEQ ID | YYARSVQY | PTFDS |
| | NO: 1) | (SEQ ID | (SEQ ID |
| | | NO: 2) | NO: 3) |
| rF1 | RFAMS | SINNGNNP | DHPSSGW |
| | (SEQ ID | YYARSVQY | PTFDS |
| | NO: 1) | (SEQ ID | (SEQ ID |
| | | NO: 2) | NO: 3) |
| rF1.v1 | RFAMS | SINSGNNP | DHPSSGW |
| | (SEQ ID | YYARSVQY | PTFDS |
| | NO: 1) | (SEQ ID | (SEQ ID |
| | | NO: 8) | NO: 3) |

TABLE 4B
Light chain CDR sequences

| Antibody | LC CDR1 | LC CDR2 | LC CDR3 |
|---|---|---|---|
| F1 | RASENVGDWLA | KTSILES | QHYIRFPYT |
| | (SEQ ID NO: 4) | (SEQ ID NO: 5) | (SEQ ID NO: 6) |
| rF1 | RASENVGDWLA | KTSILES | QHYMRFPYT |
| | (SEQ ID NO: 4) | (SEQ ID NO: 5) | (SEQ ID NO: 7) |
| rF1.v6 | RASENVGDWLA | KTSILES | QHYIRFPYT |
| | (SEQ ID NO: 4) | (SEQ ID NO: 5) | (SEQ ID NO: 6) |

In one embodiment, the H and L chain Framework (FR) sequences are as follows:

HC FW1
(SEQ ID NO. 16)
EVQLVESGGGLVQPGGSLRLSCAASGFTLS
HC FW2
(SEQ ID NO. 17)
WVRQAPGRGLEWVA
HC FW3
(SEQ ID NO. 18)
RFTVSRDVSQNTVSLQMNNLRAEDSATYFCAK
HC FW4
(SEQ ID NO. 19)
WGPGTLVTVSS
LC FW1
(SEQ ID NO. 20)
DIQLTQSPSALPASVGDRVSITC
LC FW2
(SEQ ID NO. 21)
WYRQKPGKAPNLLIY
LC FW3
(SEQ ID NO. 22)
GVPSRFSGSGSGTEFTLTISSLQPDDFATYYC
LC FW4
(SEQ ID NO. 23)
FGQGTKVEIKRTV

Various amino acid modifications were made to rF1 to improve stability and function. In the HC CDR2, the NG deamindation site was eliminated by changing the 4th residue N to S, thus improving the stability of the antibody. A repair of TV was made to the LC backbone to eliminate the severe antibody aggregation present in rF1.

For conjugation to form the therapeutic AACs of the invention, the following pairings of H and L chain can be made to form the full tetrameric antibody. Boxed are the CDR1, CDR2, CDR3 sequences. The introduced Cysteine (C) is underlined. Residues in bold are amino acid changes over the parent F1. In the L chain, the A after the bolded “RTV” is the first residue of the Constant region. The underlined C at Kabat position 114 in the H chain starts the Constant region.

In 1A and 2A, the full length (FL) L chain of SEQ ID NO. 9 with an engineered Cys at aa 205 near the end of C kappa is paired with the FL IgG1 H chain of SEQ ID NO. 10 (no Cys). This antibody will have 2 Cys sites, one on each L chain, for conjugation to the linker-antibiotic unit to form the AAC.

1A. rF1-V205C FL Light chain
(SEQ ID NO.9)

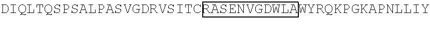

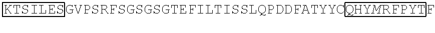

GQGTKVEIKRTVAAPSVFIFPPSDEQLKSGTASVVCLLNNFYPREAKV
QWKVDNALQSGNSQESVTEQDSKDSTYSLSSTLTLSKADYEKHKVYAC
EVTHQGLSSPCTKSFNRGEC
2A. rF1.v1 FL Heavy chain (No Cys), pair of rF1-
V205C Light Chain with Cys205
(SEQ ID NO: 10)

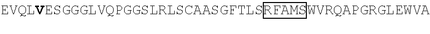

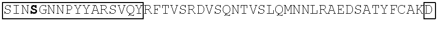

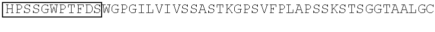

LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRIPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

In 1B with 2A, rF1.v6 L chain of SEQ ID NO. 11 with an engineered Cys 205 is paired with the FL IgG1 H chain of SEQ ID NO. 10 (no Cys). This antibody will have 2 Cys sites, one on each L chain, for conjugation to the linker-antibiotic unit.

1B. rF1.v6-V205C Light chain
(FL SEQ ID NO. 11)

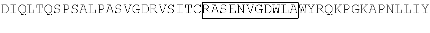

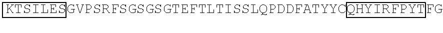

QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEV
THQGLSSPCTKSFNRGEC

In 1B with 2B, each of L and H chains has an engineered Cys, thus the tetramer antibody can have up to 4 AAR (Antibiotic: antibody ratio).

2B rF1.v1 FL Heavy chain, with Cys114
(114 Kabat numbering, or 118 -Eu numbering)
(SEQ ID NO. 12)
EVQLVESGGGLVQPGGSLRLSCAASGFTLSRFAMSWVRQAPGRGLEWVA
SINSGNNPYYARSVQYRFTVSRDVSQNTVSLQMNNLRAEDSATYFCAKD
HPSSGWPTFDSWGPGTLVTVSSCSTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSREEMTKNQVSLICLVKGFYPSDIAVEWESNGQP
ENNYKTTPPVLDSDGSFFLYSKLTVDKSRWQQGNVFSCSVMHEALHNHY
TQKSLSLSPG
rF1.v1 H chain Variable region
(SEQ ID NO. 13)

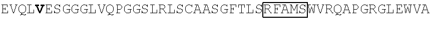

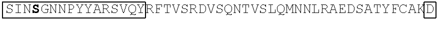

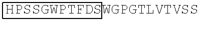

rF1 L chain Variable region
(SEQ ID NO. 14)

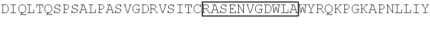

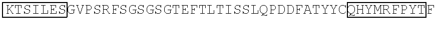

GQGTKVEIKRTV
rF1.v6 L chain Variable region
(SEQ ID NO. 15)

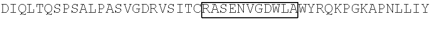

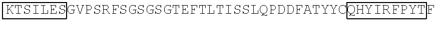

GQGTKVEIKRTV

The anti-SDR Abs including rF1 may comprise at least one amino acid other than cysteine has been replaced with cysteine. In some embodiments, the at least one amino acid other than cysteine is valine at light chain position 205 and/or valine at light chain position 110, and/or alanine at heavy chain position 114, whereby the amino acid numbering is according to Kabat (1991), which is the same as position 118 according to the Eu numbering convention.

### Rifamycin-Type Antibiotic Moieties

The antibiotic moiety (abx) of the antibody-antibiotic conjugates (AAC) of the invention is a rifamycin-type antibiotic or group that has a cytotoxic or cytostatic effect. The rifamycins are a group of antibiotics that are obtained either naturally by the bacterium, Nocardia mediterranei, Amycolatopsis mediterranei or artificially. They are a subclass of the larger Ansamycin family which inhibit bacterial RNA polymerase (Fujii et al (1995) Antimicrob. Agents Chemother. 39:1489-1492; Feklistov, et al (2008) Proc Natl Acad Sci USA, 105(39): 14820-5) and have potency against gram-positive and selective gram-negative bacteria. Rifamycins are particularly effective against mycobacteria, and are therefore used to treat tuberculosis, leprosy, and mycobacterium avium complex (MAC) infections. The rifamycin-type group includes the “classic” rifamycin drugs as well as the rifamycin derivatives rifampicin (rifampin, CA Reg. No. 13292-46-1), rifabutin (CA Reg. No. 72559-06-9; US 2011/0178001), rifapentine and rifalazil (CA Reg. No. 129791-92-0, Rothstein et al (2003) Expert Opin. Investig. Drugs 12(2):255-271; Fujii et al (1994) Antimicrob. Agents Chemother. 38:1118-1122. Many rifamycin-type antibiotics share the detrimental property of resistance development (Wichelhaus et al (2001) J. Antimicrob. Chemother. 47:153-156). Rifamycins were first isolated in 1957 from a fermentation culture of Streptomyces mediterranei. About seven rifamycins were discovered, named Rifamycin A, B, C, D, E, S, and SV (U.S. Pat. No. 3,150,046). Rifamycin B was the first introduced commercially and was useful in treating drug-resistant tuberculosis in the 1960s. Rifamycins have been used for the treatment of many diseases, the most important one being HIV-related Tuberculosis. Due to the large number of available analogues and derivatives, rifamycins have been widely utilized in the elimination of pathogenic bacteria that have become resistant to commonly used antibiotics. For instance, Rifampicin is known for its potent effect and ability to prevent drug resistance. It rapidly kills fast-dividing bacilli strains as well as “persisters” cells, which remain biologically inactive for long periods of time that allow them to evade antibiotic activity. In addition, rifabutin and rifapentine have both been used against tuberculosis acquired in HIV-positive patients.

Antibiotic moieties (abx) of the Formula I antibody-antibiotic conjugates are rifamycin-type moieties having the structure:

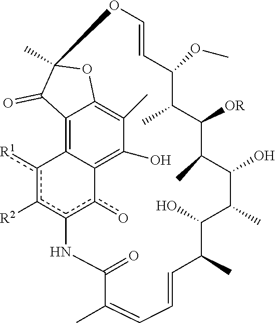

wherein:

the dashed lines indicate an optional bond;

R is H, C₁-C₁₂ alkyl, or C(O)CH₃;

R¹ is OH;

R² is CH═N-(heterocyclyl), wherein the heterocyclyl is optionally substituted with one or more groups independently selected from C(O)CH₃, C₁-C₁₂ alkyl, C₁-C₁₂ heteroaryl, C₂-C₂₀ heterocyclyl, C₆-C₂₀ aryl, and C₃-C₁₂ carbocyclyl;

or R¹ and R² form a five- or six-membered fused heteroaryl or heterocyclyl, and optionally forming a spiro or fused six-membered heteroaryl, heterocyclyl, aryl, or carbocyclyl ring, wherein the spiro or fused six-membered heteroaryl, heterocyclyl, aryl, or carbocyclyl ring is optionally substituted H, F, Cl, Br, I, C₁-C₁₂ alkyl, or OH; and

where the non-peptide linker PML is covalently attached to R².

An embodiment of a rifamycin-type moiety is:

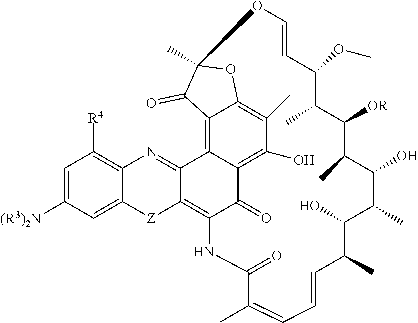

wherein R³ is independently selected from H and C₁-C₁₂ alkyl; R⁴ is selected from H, F, Cl, Br, I, C₁-C₁₂ alkyl, and OH; and Z is selected from NH, N(C₁-C₁₂ alkyl), O and S; and where the non-peptide linker PML is covalently attached to the nitrogen atom of N(R³)₂.

An embodiment of a rifampicin-type moiety is:

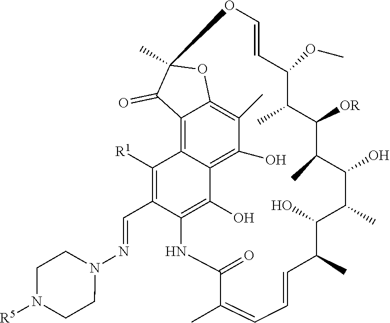

wherein

R⁵ is selected from H and C₁-C₁₂ alkyl; and where the non-peptide linker PML is covalently attached to the nitrogen atom of NR⁵.

An embodiment of a rifabutin-type moiety is:

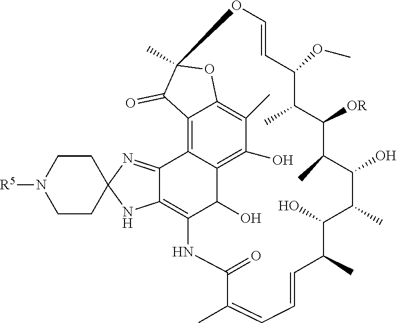

wherein R⁵ is selected from H and C₁-C₁₂ alkyl; and where the non-peptide linker PML is covalently attached to the nitrogen atom of NR⁵.

An embodiment of a benzoxazinorifamycin-type moiety is:

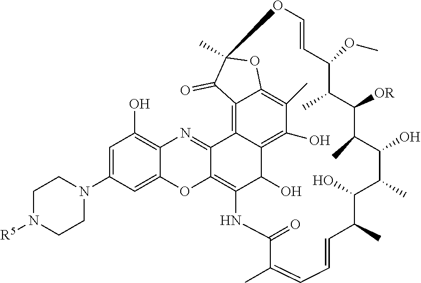

wherein R⁵ is selected from H and C₁-C₁₂ alkyl; and where the non-peptide linker PML is covalently attached to the nitrogen atom of NR⁵.

An embodiment of a benzoxazinorifamycin-type moiety, referred to herein as pipBOR, is:

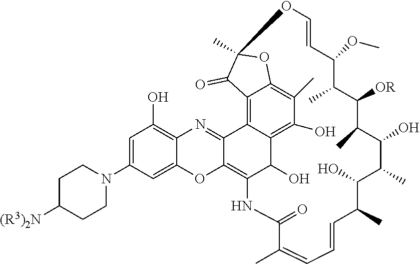

wherein R³ is independently selected from H and C₁-C₁₂ alkyl; and where the non-peptide linker PML is covalently attached to the nitrogen atom of N(R³)₂.

An embodiment of a benzoxazinorifamycin-type moiety, referred to herein as dimethylpipBOR, is:

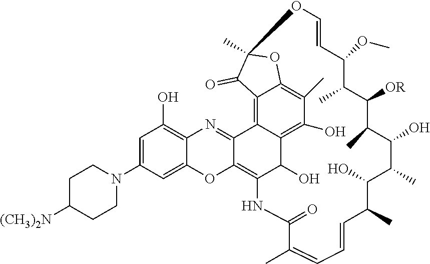

where the non-peptide linker PML is covalently attached to the nitrogen atom of N(CH₃)₂.

The semi-synthetic derivative rifamycin S, or the reduced, sodium salt form rifamycin SV, can be converted to Rifalazil-type antibiotics in several steps, where R is H, or Ac, R³ is independently selected from H and C₁-C₁₂ alkyl; R⁴ is selected from H, F, Cl, Br, I, C₁-C₁₂ alkyl, and OH; and Z is selected from NH, N(C₁-C₁₂ alkyl), O and S (see, e.g., FIG. 23A and B, and FIG. 25A and B in WO 2014/194247). Benzoxazino (Z=O), benzthiazino (Z=S), benzdiazino (Z=NH, N(C₁-C₁₂ alkyl) rifamycins may be prepared (U.S. Pat. No. 7,271,165). Benzoxazinorifamycin (BOR), benzthiazinorifamycin (BTR), and benzdiazinorifamycin (BDR) analogs that contain substituents are numbered according to the numbering scheme provided in formula A at column 28 in U.S. Pat. No. 7,271,165, which is incorporated by reference for this purpose. By “25-O-deacetyl” rifamycin is meant a rifamycin analog in which the acetyl group at the 25-position has been removed. Analogs in which this position is further derivatized are referred to as a “25-O-deacetyl-25-(substituent) rifamycin”, in which the nomenclature for the derivatizing group replaces “substituent” in the complete compound name.

Rifamycin-type antibiotic moieties can be synthesized by methods analogous to those disclosed in U.S. Pat. No. 4,610,919; U.S. Pat. No. 4,983,602; U.S. Pat. No. 5,786,349; U.S. Pat. No. 5,981,522; U.S. Pat. No. 4,859,661; U.S. Pat. No. 7,271,165; US 2011/0178001; Seligson, et al., (2001) Anti-Cancer Drugs 12:305-13; Chem. Pharm. Bull., (1993) 41:148, and in WO 2014/194247, each of which is hereby incorporated by reference). Rifamycin-type antibiotic moieties can be screened for antimicrobial activity by measuring their minimum inhibitory concentration (MIC), using standard MIC in vitro assays (Tomioka et al., (1993) Antimicrob. Agents Chemother. 37:67).

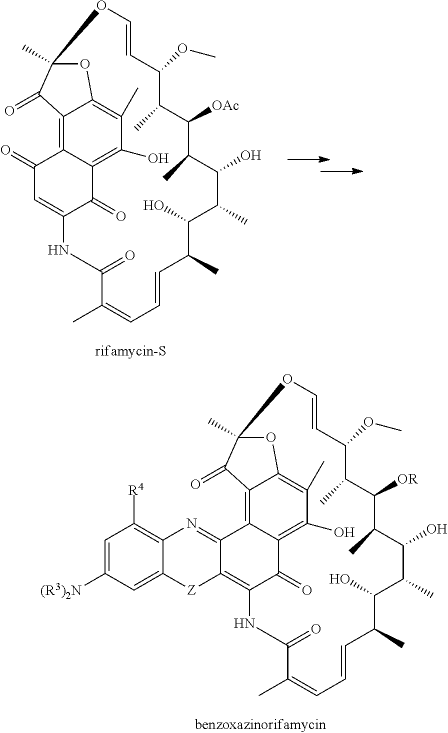

### Protease-Cleavable Non-Peptide Linkers

A “protease-cleavable, non-peptide linker” (PML) is a bifunctional or multifunctional moiety which is covalently attached to one or more antibiotic moieties (abx) and an antibody unit (Ab) to form antibody-antibiotic conjugates (AAC) of Formula I. Protease-cleavable, non-peptide linkers in AAC are substrates for cleavage by intracellular proteases, including under lysosomal conditions. Proteases includes various cathepsins and caspases. Cleavage of the non-peptide linker of an AAC inside a cell may release the rifamycin-type antibiotic with antibacterial effects.

Antibody-antibiotic conjugates (AAC) can be conveniently prepared using a linker reagent or linker-antibiotic intermediate having reactive functionality for binding to the antibiotic (abx) and to the antibody (Ab). In one exemplary embodiment, a cysteine thiol of a cysteine engineered antibody (Ab) can form a bond with a functional group of a linker reagent, an antibiotic moiety or antibiotic-linker intermediate.

The PML moiety of an AAC may comprise one amino acid residue.

The PML moiety of an AAC comprises a peptidomimetic unit.

In one aspect, a linker reagent or linker-antibiotic intermediate has a reactive site which has an electrophilic group that is reactive to a nucleophilic cysteine present on an antibody. The cysteine thiol of the antibody is reactive with an electrophilic group on a linker reagent or linker-antibiotic, forming a covalent bond. Useful electrophilic groups include, but are not limited to, maleimide and haloacetamide groups.

Cysteine engineered antibodies react with linker reagents or linker-antibiotic intermediates, with electrophilic functional groups such as maleimide or α-halo carbonyl, according to the conjugation method at page 766 of Klussman, et al (2004), Bioconjugate Chemistry 15(4):765-773, and according to the protocol of Example 18.

In another embodiment, the reactive group of a linker reagent or linker-antibiotic intermediate contains a thiol-reactive functional group that can form a bond with a free cysteine thiol of an antibody. Examples of thiol-reaction functional groups include, but are not limited to, maleimide, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates and isothiocyanates.

In another embodiment, a linker reagent or antibiotic-linker intermediate has a reactive functional group which has a nucleophilic group that is reactive to an electrophilic group present on an antibody. Useful electrophilic groups on an antibody include, but are not limited to, pyridyl disulfide, aldehyde and ketone carbonyl groups. The heteroatom of a nucleophilic group of a linker reagent or antibiotic-linker intermediate can react with an electrophilic group on an antibody and form a covalent bond to an antibody unit. Useful nucleophilic groups on a linker reagent or antibiotic-linker intermediate include, but are not limited to, hydrazide, oxime, amino, thiol, hydrazine, thiosemicarbazone, hydrazine carboxylate, and arylhydrazide. The electrophilic group on an antibody provides a convenient site for attachment to a linker reagent or antibiotic-linker intermediate.

A PML moiety may comprise one or more linker components. Exemplary linker components include a single amino acid such as citrulline (“cit”), 6-maleimidocaproyl (“MC”), maleimidopropanoyl (“MP”), and p-aminobenzyloxycarbonyl (“PAB”), N-succinimidyl 4-(2-pyridylthio) pentanoate (“SPP”), and 4-(N-maleimidomethyl) cyclohexane-1 carboxylate (“MCC”). Various linker components are known in the art, some of which are described below.

In another embodiment, the linker may be substituted with groups that modulate solubility or reactivity. For example, a charged substituent such as sulfonate (—SO3-) or ammonium, may increase water solubility of the reagent and facilitate the coupling reaction of the linker reagent with the antibody or the antibiotic moiety, or facilitate the coupling reaction of Ab-L (antibody-linker intermediate) with abx, or abx-L (antibiotic-linker intermediate) with Ab, depending on the synthetic route employed to prepare the AAC.

The AAC of the invention expressly contemplate, but are not limited to, those prepared with linker reagents: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, SVSB (succinimidyl-(4-vinylsulfone)benzoate), and bis-maleimide reagents such as DTME, BMB, BMDB, BMH, BMOE, BM(PEG)2, and BM(PEG)3. Bis-maleimide reagents allow the attachment of the thiol group of a cysteine engineered antibody to a thiol-containing antibiotic moiety, label, or linker intermediate, in a sequential or convergent fashion. Other functional groups besides maleimide, which are reactive with a thiol group of a cysteine engineered antibody, antibiotic moiety, or linker-antibiotic intermediate include iodoacetamide, bromoacetamide, vinyl pyridine, disulfide, pyridyl disulfide, isocyanate, and isothiocyanate.

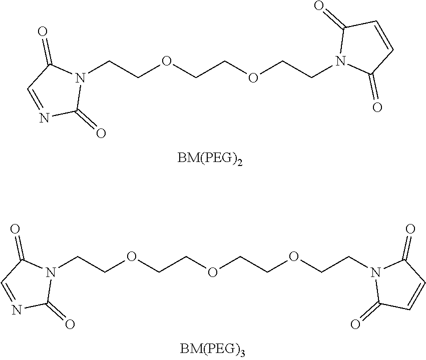 [SMILES: O=C1C=CC(=O)N1CCOCCOCCN1C(=O)C=NC1=O ; O=C1C=CC(=O)N1CCOCCOCCOCCN1C(=O)C=NC1=O]

Useful linker reagents can also be obtained via other commercial sources, such as Molecular Biosciences Inc. (Boulder, Colo.), or synthesized in accordance with procedures described in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

In another embodiment, the PML moiety of an AAC comprises a dendritic type linker for covalent attachment of more than one antibiotic moiety through a branching, multifunctional linker moiety to an antibody (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of antibiotic to antibody, i.e. loading, which is related to the potency of the AAC. Thus, where a cysteine engineered antibody bears only one reactive cysteine thiol group, a multitude of antibiotic moieties may be attached through a dendritic linker.

In certain embodiments of Formula I AAC, the protease-cleavable, non-peptide linker PML has the formula:

-Str-PM-Y—

where Str is a stretcher unit; PM is a peptidomimetic unit, and Y is a spacer unit;

abx is the rifamycin-type antibiotic; and

p is an integer from 1 to 8.

In one embodiment, a stretcher unit “Str” has the formula:

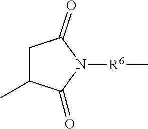

wherein R⁶ is selected from the group consisting of C₁-C₁₂ alkylene, C₁-C₁₂ alkylene-C(═O), C₁-C₁₂ alkylene-NH, (CH₂CH₂O)ᵣ, (CH₂CH₂O)ᵣ—C(═O), (CH₂CH₂O)ᵣ—CH₂, and C₁-C₁₂ alkylene-NHC(═O)CH2CH(thiophen-3-yl), where r is an integer ranging from 1 to 10.

Exemplary stretcher units are shown below (wherein the wavy line indicates sites of covalent attachment to an antibody):

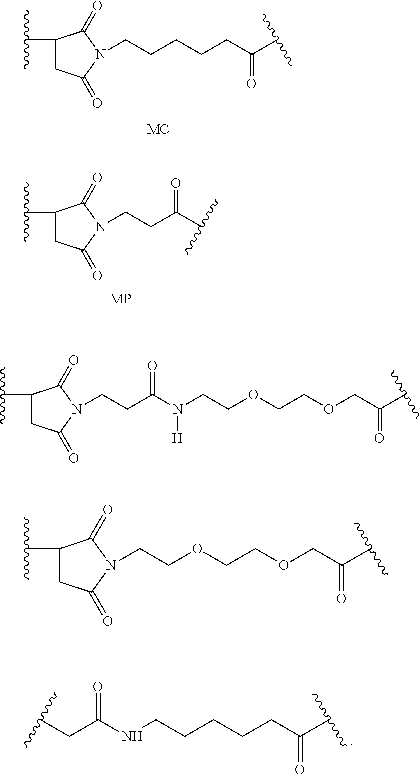 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *C(=O)COCCOCCN1C(=O)CC(*)C1=O ; *CC(=O)NCCCCCC(*)=O]

In one embodiment, PM has the formula:

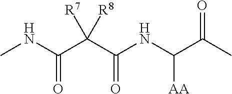

where R⁷ and R⁸ together form a C3-C7 cycloalkyl ring, and

AA is an amino acid side chain selected from H, —CH₃, —CH₂(C₆H₅), —CH₂CH₂CH₂CH₂NH₂, —CH₂CH₂CH₂NHC(NH)NH₂, —CHCH(CH₃)CH₃, and —CH₂CH₂CH₂NHC(O)NH₂.

In one embodiment, spacer unit Y comprises para-aminobenzyl (PAB) or para-aminobenzyloxycarbonyl (PABC).

A spacer unit allows for release of the antibiotic moiety without a separate hydrolysis step. A spacer unit may be “self-immolative” or a “non-self-immolative.” In certain embodiments, a spacer unit of a linker comprises a p-aminobenzyl unit (PAB). In one such embodiment, a p-aminobenzyl alcohol is attached to an amino acid unit via an amide bond, a carbamate, methylcarbamate, or carbonate between the p-aminobenzyl group and the antibiotic moiety (Hamann et al. (2005) Expert Opin. Ther. Patents (2005) 15:1087-1103). In one embodiment, the spacer unit is p-aminobenzyloxycarbonyl (PAB).

In one embodiment, the antibiotic comprises a quaternary amine, such as the dimethylaminopiperidyl group, when attached to the PAB spacer unit of the non-peptide linker. Examples of such quaternary amines are linker-antibiotic intermediates (PLA) are PLA-1 to 4 from Table 2. The quaternary amine group may modulate cleavage of the antibiotic moiety to optimize the antibacterial effects of the AAC. In another embodiment, the antibiotic is linked to the PABC spacer unit of the non-peptide linker, forming a carbamate functional group in the AAC. Such carbamate functional group may also optimize the antibacterial effects of the AAC. Examples of PABC carbamate linker-antibiotic intermediates (PLA) are PLA-5 and PLA-6 from Table 2.

Other examples of self-immolative spacers include, but are not limited to, aromatic compounds that are electronically similar to the PAB group such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. Spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Elimination of amine-containing drugs that are substituted at glycine (Kingsbury et al (1984) J. Med. Chem. 27:1447) is also exemplary of self-immolative spacers useful in AAC.

The amount of active antibiotic released from cleavage of AAC can be measured by a caspase release assay.

### Linker-Antibiotic Intermediates Useful for AAC

PML Linker-antibiotic intermediates (PLA) of Formula II and Table 2 were prepared by coupling a rifamycin-type antibiotic moiety with a linker reagent, Examples 7-17. Linker reagents were prepared by methods described in WO 2012/113847; U.S. Pat. No. 7,659,241; U.S. Pat. No. 7,498,298; US 20090111756; US 2009/0018086; U.S. Pat. No. 6,214,345; Dubowchik et al (2002) Bioconjugate Chem. 13(4):855-869

TABLE 2
PML Linker-antibiotic intermediates

| LA | |
|---|---|
| No. | Structure |
| PLA-1 | 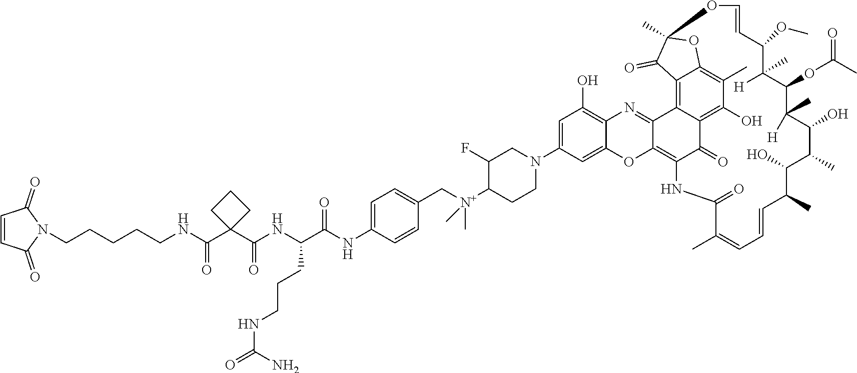 [SMILES: CO[C@H]1/C=C/O[C@@]2(C)Oc3c(C)c(O)c4c(=O)c(c5oc6cc(N7CCC([N+](C)(C)Cc8ccc(NC(=O)[C@H](CCCNC(N)=O)NC(=O)C9(C(=O)NCCCCCN%10C(=O)C=CC%10=O)CCC9)cc8)C(F)C7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| PLA-2 | 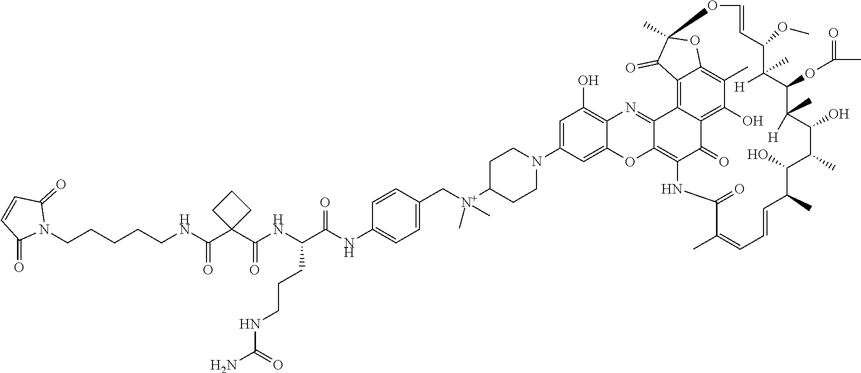 [SMILES: CO[C@H]1/C=C/O[C@@]2(C)Oc3c(C)c(O)c4c(=O)c(c5oc6cc(N7CCC([N+](C)(C)Cc8ccc(NC(=O)[C@H](CCCNC(N)=O)NC(=O)C9(C(=O)NCCCCCN%10C(=O)C=CC%10=O)CCC9)cc8)CC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| PLA-3 | 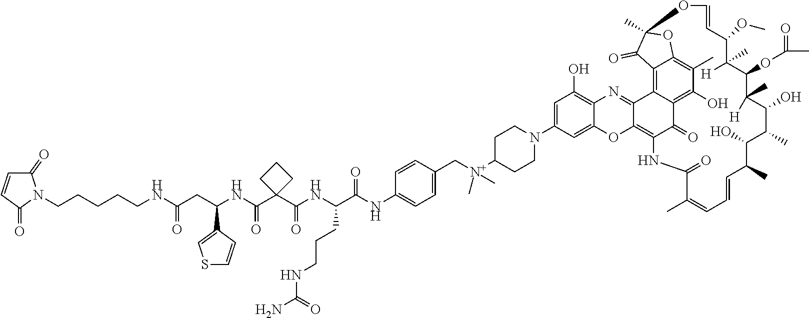 [SMILES: CO[C@H]1/C=C/O[C@@]2(C)Oc3c(C)c(O)c4c(=O)c(c5oc6cc(N7CCC([N+](C)(C)Cc8ccc(NC(=O)[C@H](CCCNC(N)=O)NC(=O)C9(C(=O)N[C@H](CC(=O)NCCCCCN%10C(=O)C=CC%10=O)c%10ccsc%10)CCC9)cc8)CC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| PLA-4 | 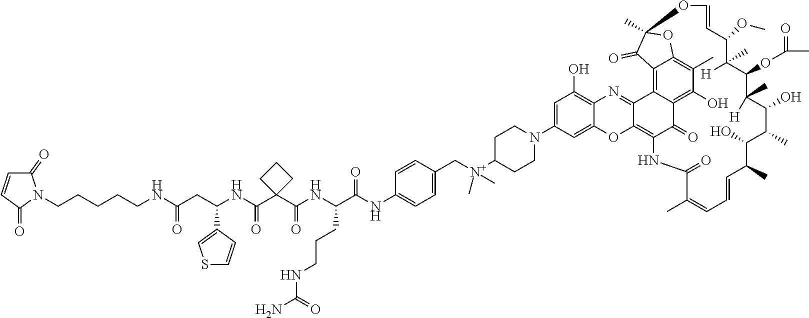 [SMILES: CO[C@H]1/C=C/O[C@@]2(C)Oc3c(C)c(O)c4c(=O)c(c5oc6cc(N7CCC([N+](C)(C)Cc8ccc(NC(=O)[C@H](CCCNC(N)=O)NC(=O)C9(C(=O)N[C@@H](CC(=O)NCCCCCN%10C(=O)C=CC%10=O)c%10ccsc%10)CCC9)cc8)CC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| PLA-5 | 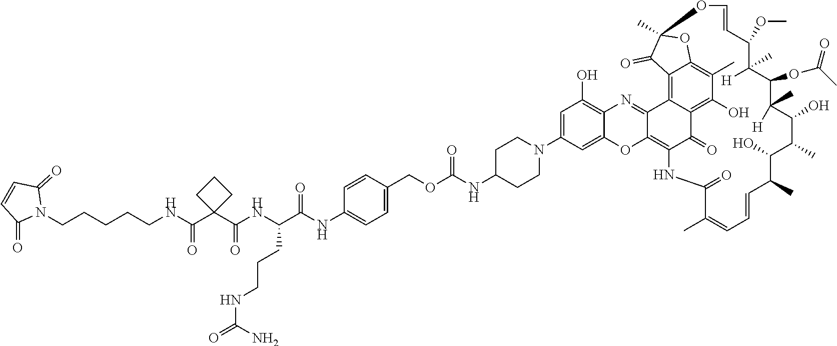 [SMILES: CO[C@H]1/C=C/O[C@@]2(C)Oc3c(C)c(O)c4c(=O)c(c5oc6cc(N7CCC(NC(=O)OCc8ccc(NC(=O)[C@H](CCCNC(N)=O)NC(=O)C9(C(=O)NCCCCCN%10C(=O)C=CC%10=O)CCC9)cc8)CC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| PLA-6 | 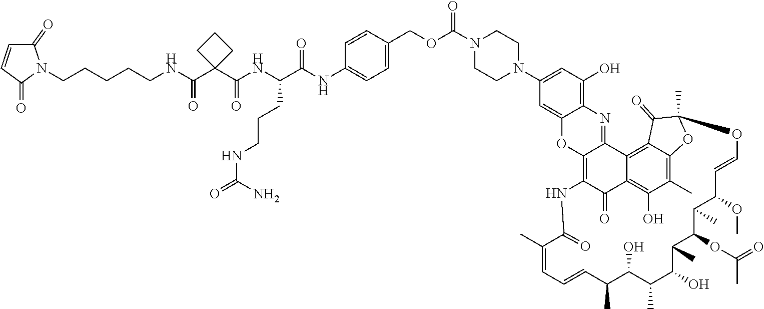 [SMILES: CO[C@H]1/C=C/O[C@@]2(C)Oc3c(C)c(O)c4c(=O)c(c5oc6cc(N7CCN(C(=O)OCc8ccc(NC(=O)[C@H](CCCNC(N)=O)NC(=O)C9(C(=O)NCCCCCN%10C(=O)C=CC%10=O)CCC9)cc8)CC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C] |

### Embodiments of Antibody-Antibiotic Conjugates

Cysteine engineered, rF1 antibodies were linked via the free cysteine thiol group to derivatives of rifamycin, termed pipBOR and others, via a protease cleavable, non-peptide linker to form the antibody-antibiotic conjugate compounds (AAC) in Table 3. The linker is designed to be cleaved by lysosomal proteases including cathepsins B, D and others, Generation of the linker-antibiotic intermediate consisting of the antibiotic and the PML linker and others, is described in detail in Examples 7-17. The linker is designed such that cleavage of the amide bond at the PAB moiety separates the antibody from the antibiotic in an active state.

The AAC named “dimethylpipBOR” is identical to the “pipBOR” AAC except for the dimethylated amino on the antibiotic and the oxycarbonyl group on the linker.

FIG. 3 shows a possible mechanism of drug activation for antibody-antibiotic conjugates (AAC). Active antibiotic (Ab) is only released after internalization of the AAC inside mammalian cells. The Fab portion of the antibody in AAC binds S. aureus whereas the Fc portion of the AAC enhances uptake of the bacteria by Fc-receptor mediated binding to phagocytic cells including neutrophils and macrophages. After internalization into the phagolysosome, the linker may be cleaved by lysosomal proteases releasing the active antibiotic inside the phagolysosome.

An embodiment of the antibody-antibiotic conjugate (AAC) compounds of the invention includes Formula I:

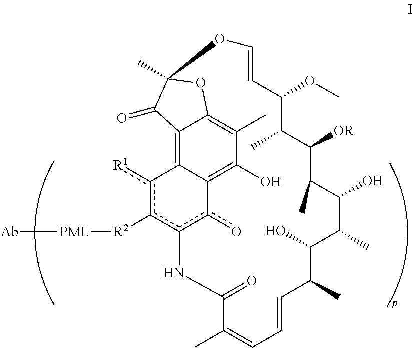

wherein:

the dashed lines indicate an optional bond;

R is H, C₁-C₁₂ alkyl, or C(O)CH₃;

R¹ is OH;

R² is CH═N-(heterocyclyl), wherein the heterocyclyl is optionally substituted with one or more groups independently selected from C(O)CH₃, C₁-C₁₂ alkyl, C₁-C₁₂ heteroaryl, C₂-C₂₀ heterocyclyl, C₆-C₂₀ aryl, and C₃-C₁₂ carbocyclyl;

or R¹ and R² form a five- or six-membered fused heteroaryl or heterocyclyl, and optionally forming a spiro or fused six-membered heteroaryl, heterocyclyl, aryl, or carbocyclyl ring, wherein the spiro or fused six-membered heteroaryl, heterocyclyl, aryl, or carbocyclyl ring is optionally substituted H, F, Cl, Br, I, C₁-C₁₂ alkyl, or OH;

PML is the protease-cleavable, non-peptide linker attached to R² or the fused heteroaryl or heterocyclyl formed by R¹ and R²;

Ab is the rF1 antibody; and

p is an integer from 1 to 8.

Another embodiment of the antibody-antibiotic conjugate (AAC) compounds of the invention includes the formula:

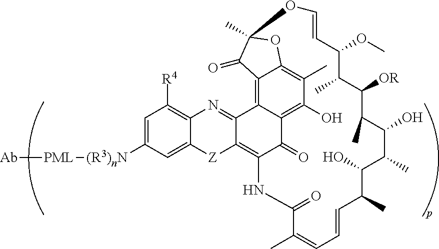

wherein

R³ is independently selected from H and C₁-C₁₂ alkyl;

n is 1 or 2;

R⁴ is selected from H, F, Cl, Br, I, C₁-C₁₂ alkyl, and OH; and

Z is selected from NH, N(C₁-C₁₂ alkyl), O and S.

Another embodiment of the antibody-antibiotic conjugate (AAC) compounds of the invention includes the formula:

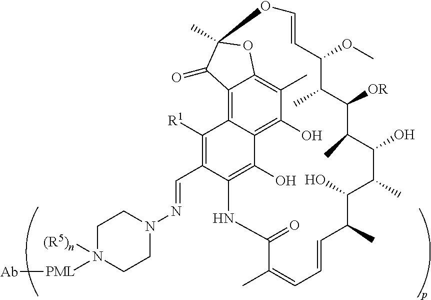

wherein

R⁵ is selected from H and C₁-C₁₂ alkyl; and

n is 0 or 1.

Another embodiment of the antibody-antibiotic conjugate (AAC) compounds of the invention includes the formula:

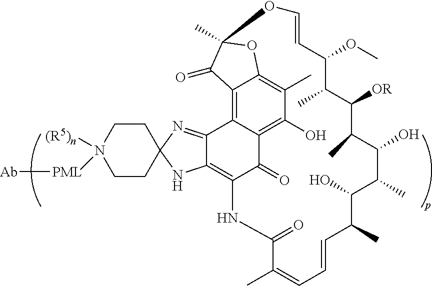

wherein

R⁵ is selected from H and C₁-C₁₂ alkyl; and

n is 0 or 1.

Another embodiment of the antibody-antibiotic conjugate (AAC) compounds of the invention includes the formula:

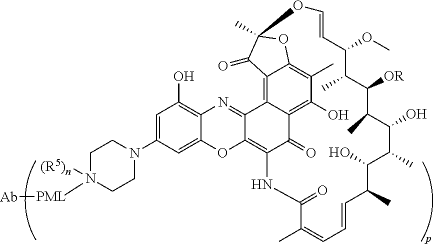

wherein

R⁵ is independently selected from H and C₁-C₁₂ alkyl; and

n is 0 or 1.

Another embodiment of the antibody-antibiotic conjugate (AAC) compounds of the invention includes the formula:

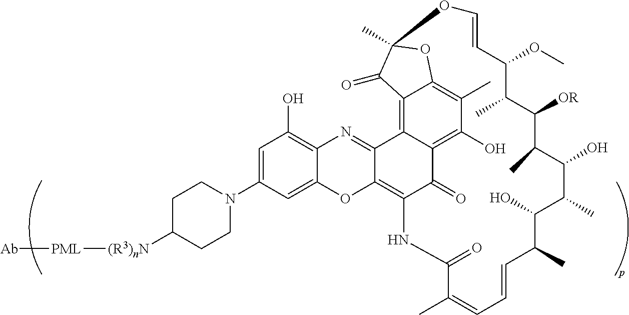

wherein

R³ is independently selected from H and C₁-C₁₂ alkyl; and

n is 1 or 2.

Another embodiment of the antibody-antibiotic conjugate (AAC) compounds of the invention includes the formula:

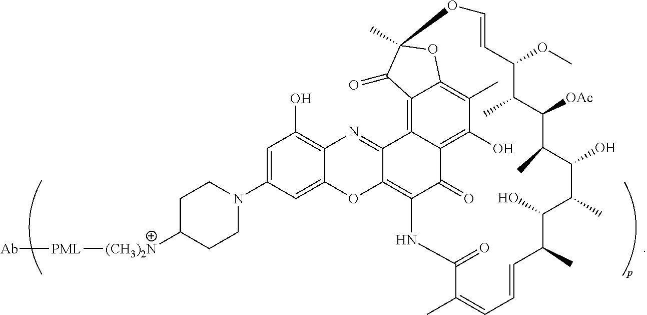

Another embodiment of the antibody-antibiotic conjugate (AAC) compounds of the invention includes the formula:

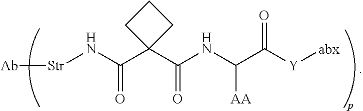

Another embodiment of the antibody-antibiotic conjugate (AAC) compounds of the invention includes the formula:

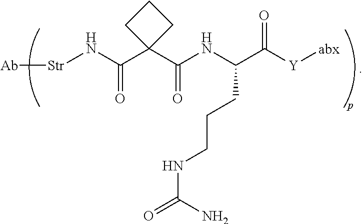

Another embodiment of the antibody-antibiotic conjugate (AAC) compounds of the invention includes the formula:

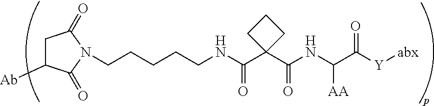

Another embodiment of the antibody-antibiotic conjugate (AAC) compounds of the invention includes the formula:

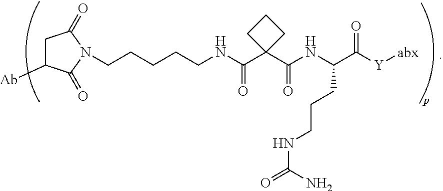

Another embodiment of the antibody-antibiotic conjugate (AAC) compounds of the invention includes the formulas:

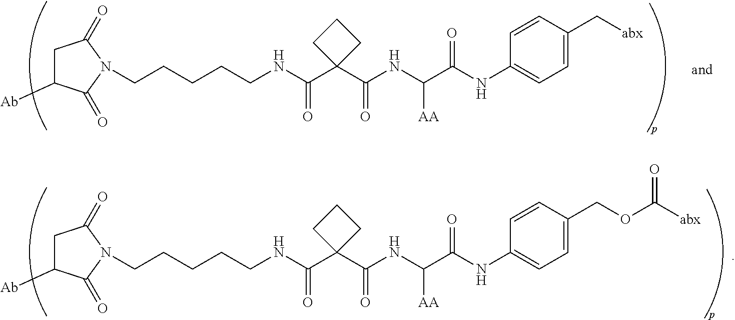

Another embodiment of the antibody-antibiotic conjugate (AAC) compounds of the invention includes the formulas:

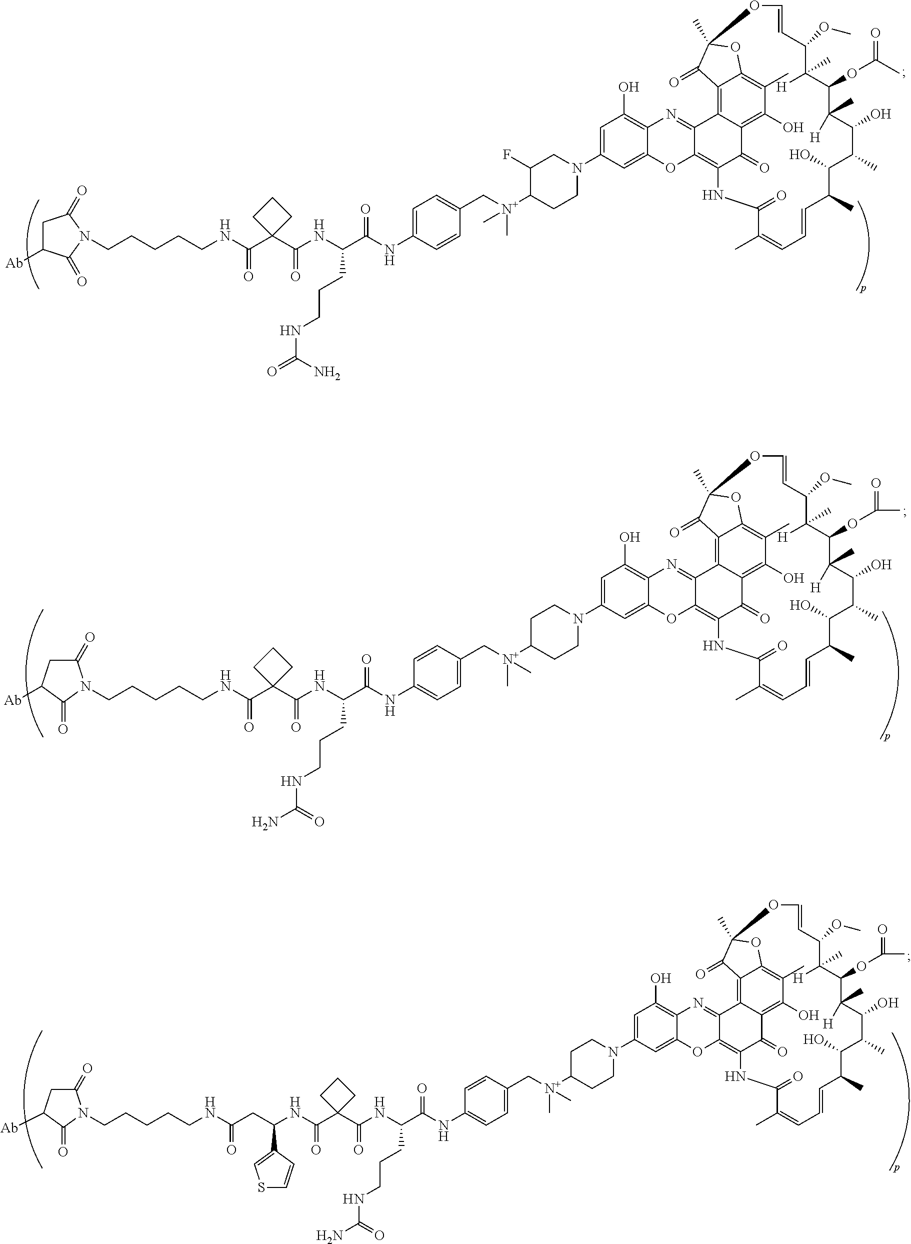

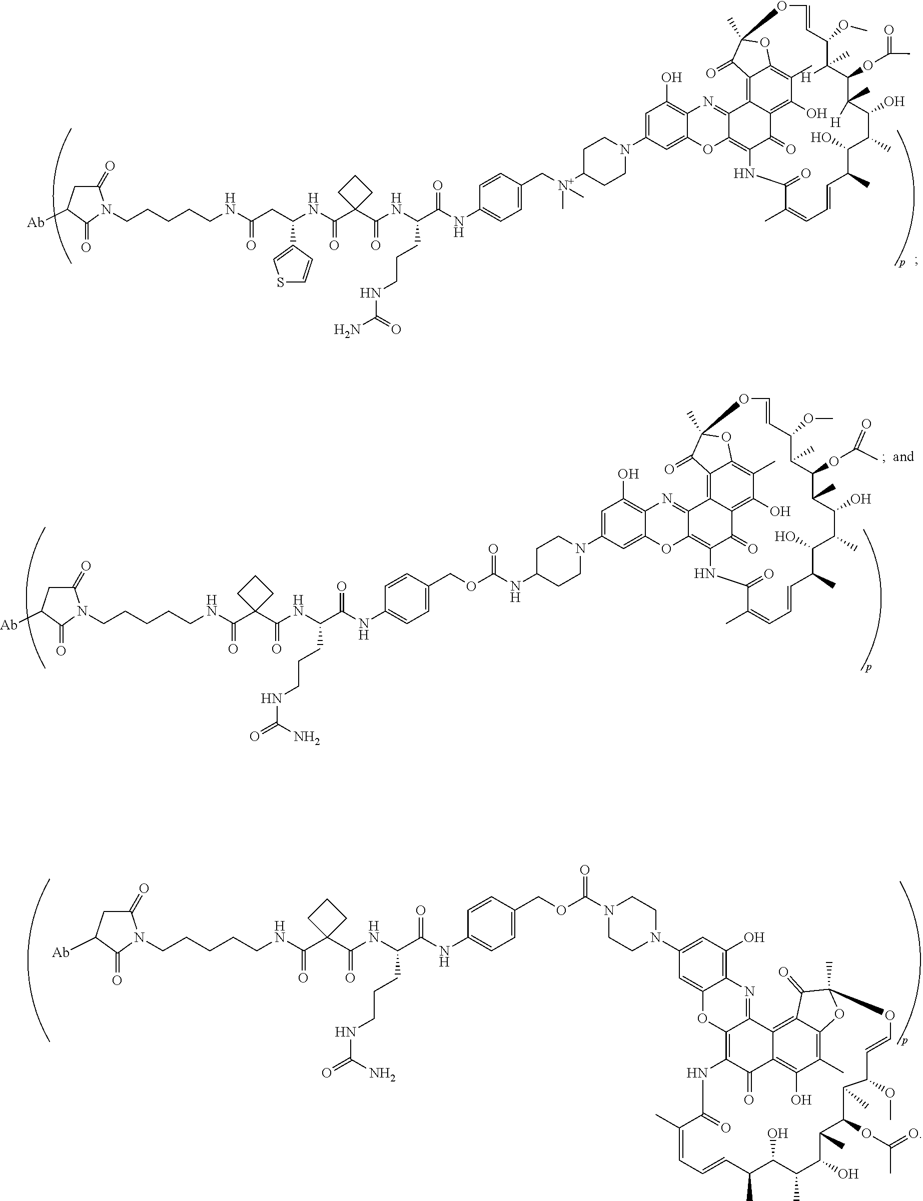

### Antibiotic Loading of AAC

Antibiotic loading is represented by p, the average number of antibiotic (abx) moieties per antibody in a molecule of Formula I. Antibiotic loading may range from 1 to 20 antibiotic moieties (D) per antibody. The AAC of Formula I include collections or a pool of antibodies conjugated with a range of antibiotic moieties, from 1 to 20. The average number of antibiotic moieties per antibody in preparations of AAC from conjugation reactions may be characterized by conventional means such as mass spectroscopy, ELISA assay, and HPLC. The quantitative distribution of AAC in terms of p may also be determined. In some instances, separation, purification, and characterization of homogeneous AAC where p is a certain value from AAC with other antibiotic loadings may be achieved by means such as reverse phase HPLC or electrophoresis.

For some antibody-antibiotic conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. In certain embodiments, higher antibiotic loading, e.g. p>5, may cause aggregation, insolubility, toxicity, or loss of cellular permeability of certain antibody-antibiotic conjugates. In certain embodiments, the antibiotic loading for an AAC of the invention ranges from 1 to about 8; from about 2 to about 6; from about 2 to about 4; or from about 3 to about 5; about 4; or about 2.

In certain embodiments, fewer than the theoretical maximum of antibiotic moieties are conjugated to an antibody during a conjugation reaction. An antibody may contain, for example, lysine residues that do not react with the antibiotic-linker intermediate or linker reagent, as discussed below. Generally, antibodies do not contain many free and reactive cysteine thiol groups which may be linked to an antibiotic moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (antibiotic/antibody ratio, “AAR”) of an AAC may be controlled in different ways, e.g., by: (i) limiting the molar excess of antibiotic-linker intermediate or linker reagent relative to antibody, (ii) limiting the conjugation reaction time or temperature, and (iii) partial or limiting reductive conditions for cysteine thiol modification. “DAR” if referred to herein or in the figures shall mean the same as “AAR”.

It is to be understood that where more than one nucleophilic group reacts with an antibiotic-linker intermediate or linker reagent followed by antibiotic moiety reagent, then the resulting product is a mixture of AAC compounds with a distribution of one or more antibiotic moieties attached to an antibody. The average number of antibiotics per antibody may be calculated from the mixture by a dual ELISA antibody assay, which is specific for antibody and specific for the antibiotic. Individual AAC molecules may be identified in the mixture by mass spectroscopy and separated by HPLC, e.g. hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous AAC with a single loading value may be isolated from the conjugation mixture by electrophoresis or chromatography. Cysteine-engineered antibodies of the invention enable more homogeneous preparations since the reactive site on the antibody is primarily limited to the engineered cysteine thiol. In one embodiment, the average number of antibiotic moieties per antibody is in the range of about 1 to about 20. In some embodiments the range is selected and controlled from about 1 to 4.

### Methods of Preparing Antibody-Antibiotic Conjugates

An AAC of Formula I may be prepared by several routes employing organic chemistry reactions, conditions, and reagents known to those skilled in the art, including: (1) reaction of a nucleophilic group of an antibody with a bivalent linker reagent to form Ab-L via a covalent bond, followed by reaction with an antibiotic moiety (abx); and (2) reaction of a nucleophilic group of an antibiotic moiety with a bivalent linker reagent, to form L-abx, via a covalent bond, followed by reaction with a nucleophilic group of an antibody. Exemplary methods for preparing an AAC of Formula I via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies through modification of lysine residues, e.g., by reacting lysine residues with 2-iminothiolane (Traut's reagent), resulting in conversion of an amine into a thiol. Reactive thiol groups may be introduced into an antibody by introducing one, two, three, four, or more cysteine residues (e.g., by preparing variant antibodies comprising one or more non-native cysteine amino acid residues).

Antibody-antibiotic conjugates of the invention may also be produced by reaction between an electrophilic group on an antibody, such as an aldehyde or ketone carbonyl group, with a nucleophilic group on a linker reagent or antibiotic. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or antibiotic. In another embodiment, the sugars of glycosylated antibodies may be oxidized, e.g. with periodate oxidizing reagents, to form aldehyde or ketone groups which may react with the amine group of linker reagents or antibiotic moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody that can react with appropriate groups on the antibiotic (Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with an antibiotic moiety or linker nucleophile.

Nucleophilic groups on an antibiotic moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

The antibody-antibiotic conjugates (AAC) in Table 3 were prepared by conjugation of the described rF1 antibodies and linker-antibiotic intermediates of Table 2, and according to the described methods in Example 18. AAC were tested for efficacy by in vitro macrophage assay (Example 19) and in vivo mouse kidney model (Example 20).

TABLE 3
rF1 Antibody-PML-antibiotic conjugates (AAC)

| AAC | | linker-abx | |
|---|---|---|---|
| No. | AAC formula | PLA No. | AAR* |
| 101 | thio-rF1-LC-V205C-MC-(CBDK-cit)-PAB- | PLA-1 | 2.0 |
| | (dimethyl, fluoropipBOR) | | |
| 102 | thio-rF1-HC-121C, LC-V205C-MC-(CBDK- | PLA-2 | 3.9 |
| | cit)-PAB-(dimethylpipBOR) | | |
| 103 | thio-rF1-LC-V205C-MC-(CBDK-cit)-PAB- | PLA-2 | 1.9 |
| | (dimethylpipBOR) | | |
| 104 | thio-rF1-HC-A121C, LC-V205C-MC- | PLA-2 | 3.7 |
| | (CBDK-cit)-PAB-(dimethylpipBOR) | | |

*AAR = antibiotic/antibody ratio average

Wild-type (“WT”), cysteine engineered mutant antibody (“thio”), light chain (“LC”), heavy chain (“HC”), 6-maleimidocaproyl (“MC”), maleimidopropanoyl (“MP”), cyclobutyldiketo (“CBDK”), citrulline (“cit”), cysteine (“cys”), p-aminobenzyl (“PAB”), and p-aminobenzyloxycarbonyl (“PABC”)

Methods of Treating and Preventing Infections with Antibody-Antibiotic Conjugates

The rF1-AAC of the invention are useful as antimicrobial agents effective against human and veterinary Staphylococci, for example S. aureus, S. saprophyticus and S. simulans. In a specific aspect, the AAC of the invention are useful to treat S. aureus infections.

Following entry into the bloodstream, S. aureus can cause metastatic infection in almost any organ. Secondary infections occur in about one-third of cases before the start of therapy (Fowler et al., (2003) Arch. Intern. Med. 163:2066-2072), and even in 10% of patients after the start of therapy (Khatib et al., (2006) Scand. J. Infect. Dis., 38:7-14). Hallmarks of infections are large reservoirs of pus, tissue destruction, and the formation of abcesses (all of which contain large quantities of neutrophils). About 40% of patients develop complications if the bacteremia persists beyond three days.

The proposed mechanism of action of an AAC has been described above (under subheading Antibody-Antibiotic Conjugates). The rF1 antibody-antibiotic conjugates (AAC) of the invention have significant therapeutic advantages for treating intracellular pathogens. The AAC linker is cleaved by exposure to phagolysosomal enzymes, releasing an active antibiotic. Due to the confined space and relatively high local antibiotic concentration (about 104 per bacterium), the result is that the phagolysosome no longer supports the survival of the intracellular pathogen. Because the AAC is essentially an inactive prodrug, the therapeutic index of the antibiotic can be extended relative to the free (unconjugated) form. The antibody provides pathogen specific targeting, while the cleavable linker is cleaved under conditions specific to the intracellular location of the pathogen. The effect can be both directly on the opsonized pathogen as well as other pathogens that are co-localized in the phagolysosome. Antibiotic tolerance is the ability of a disease-causing pathogen to resist killing by antibiotics and other antimicrobials and is mechanistically distinct from multidrug resistance (Lewis K (2007). “Persister cells, dormancy and infectious disease”. Nature Reviews Microbiology 5 (1): 48-56. doi:10.1038/nrmicro1557). Rather, this form of tolerance is caused by a small sub-population of microbial cells called persisters (Bigger J W (14 Oct. 1944). “Treatment of staphylococcal infections with penicillin by intermittent sterilization”. Lancet 244 (6320): 497-500). These cells are not multidrug resistant in the classical sense, but rather are dormant cells that are tolerant to antibiotic treatment that can kill their genetically identical siblings. This antibiotic tolerance is induced by a non- or extremely slow dividing physiological state. When antimicrobial treatment fails to eradicate these persister cells, they become a reservoir for recurring chronic infections. The antibody-antibiotic conjugates of the invention possess a unique property to kill these persister cells and suppress the emergence of multidrug tolerant bacterial populations.

In another embodiment, the rF1-AAC of the invention may be used to treat infection regardless of the intracellular compartment in which the pathogen survives.

In another embodiment, rF1-AACs of the invention could also be used to target Staphylococci bacteria in planktonic or biofilm form. Bacterial infections treatable with antibody-antibiotic conjugates (AAC) of the invention include treating bacterial pulmonary infections, such as S. aureus pneumonia, osteomyelitis, recurrent rhinosinusitis, bacterial endocarditis, bacterial ocular infections, such as trachoma and conjunctivitis, heart, brain or skin infections, infections of the gastrointestinal tract, such as travellers' diarrhea, ulcerative colitis, irritable bowel syndrome (IBS), Crohn's disease, and IBD (inflammatory bowel disease) in general, bacterial meningitis, and abscesses in any organ, such as muscle, liver, meninges, or lung. The bacterial infections can be in other parts of the body like the urinary tract, the bloodstream, a wound or a catheter insertion site. The AACs of the invention are useful for difficult-to-treat infections that involve biofilms, implants or sanctuary sites (e.g., osteomyelitis and prosthetic joint infections), and high mortality infections such as hospital acquired pneumonia and bacteremia. Vulnerable patient groups that can be treated to prevent Staphylococcal aureus infection include hemodialysis patients, immune-compromised patients, patients in intensive care units, and certain surgical patients. In another aspect, the invention provides a method of killing, treating, or preventing a microbial infection in an animal, preferably a mammal, and most preferably a human, that includes administering to the animal an rF1 AAC or pharmaceutical formulation of an AAC of the invention. The invention further features treating or preventing diseases associated with or which opportunistically result from such microbial infections. Such methods of treatment or prevention may include the oral, topical, intravenous, intramuscular, or subcutaneous administration of a composition of the invention. For example, prior to surgery or insertion of an IV catheter, in ICU care, in transplant medicine, with or post cancer chemotherapy, or other activities that bear a high risk of infection, the AAC of the invention may be administered to prevent the onset or spread of infection.

The bacterial infection may be caused by bacteria with an active and inactive form, and the AAC is administered in an amount and for a duration sufficient to treat both the active and the inactive, latent form of the bacterial infection, which duration is longer than is needed to treat the active form of the bacterial infection.

An aspect of the invention is a method of treating a patient infected with S. aureus and/or Listeria monocytogenes by administering a therapeutically effective amount of an rF1-AAC of the invention. The invention also contemplates a method of preventing infections by one or more of S. aureus or S. Epidermidis, or S. saprophyticus or S. simulans by administering a therapeutically effective amount of an rF1-AAC of the invention in hospital settings such as surgery, burn patient, and organ transplantation.

The patient needing treatment for a bacterial infection as determined by a physician of skill in the art may have already been, but does not need to be diagnosed with the kind of bacteria that he/she is infected with. Since a patient with a bacterial infection can take a turn for the worse very quickly, in a matter of hours, the patient upon admission into the hospital can be administered the rF1-AACs of the invention along with one or more standard of care Abx such as vancomycin or ciprofloxacin. When the diagnostic results become available and indicate the presence of, e.g., S. aureus in the infection, the patient can continue with treatment with the rF1 AAC. Therefore, in one embodiment of the method of treating a bacterial infection or specifically a S. aureus infection, the patient is administered a therapeutically effective amount of an rF1 AAC. In the methods of treatment or prevention of the present invention, an AAC of the invention can be administered as the sole therapeutic agent or in conjunction with other agents such as those described below. The AACs of the invention show superiority to vancomycin in the treatment of MRSA in pre-clinical models. Comparison of AACs to SOC can be measured, e.g., by a reduction in mortality rate. The patient being treated would be assessed for responsiveness to the AAC treatment by a variety of measurable factors. Examples of signs and symptoms that clinicians might use to assess improvement in their patients includes the following: normalization of the white blood cell count if elevated at diagnosis, normalization of body temperature if elevated (fever) at the time of diagnosis, clearance of blood cultures, visual improvement in wound including less erythema and drainage of pus, reduction in ventilator requirements such as requiring less oxygen or reduced rate of ventilation in a patient who is ventilated, coming off of the ventilator entirely if the patient is ventilated at the time of diagnosis, use of less medications to support a stable blood pressure if these medications were required at the time of diagnosis, normalization of lab abnormalities that suggest end-organ failure such as elevated creatinine or liver function tests if they were abnormal at the time of diagnosis, and improvement in radiologic imaging (e.g. chest x-ray that previously suggested pneumonia showing resolution). In a patient in the ICU, these factors might be measured at least daily. Fever is monitored closely as is white blood cell count including absolute neutrophil counts as well as evidence that a “left shift” (appearance of blasts indicating increased neutrophil production in response to an active infection) has resolved.

In the context of the present methods of treatment of the invention, a patient with a bacterial infection is considered to be treated if there is significant measurable improvement as assessed by the physician of skill in the art, in at least two or more of the preceding factors compared to the values, signs or symptoms before or at the start of treatment or at the time of diagnosis. In some embodiments, there is measurable improvement in 3, 4, 5, 6 or more of the aforementioned factors. If some embodiments, the improvement in the measured factors is by at least 50%, 60%, 70%, 80%, 90%, 95% or 100% compared to the values before treatment. Typically, a patient can be considered completely treated of the bacterial infection (e.g., S. aureus infection) if the patient's measurable improvements include the following: i) repeat blood or tissue cultures (typically several) that do not grow out the bacteria that was originally identified; ii) fever is normalized; iii) WBC is normalized; and iv) evidence that end-organ failure (heart, lungs, liver, kidneys, vascular collapse) has resolved either fully or partially given the pre-existent co-morbidities that the patient had.

Dosing. In any of the foregoing aspects, in treating an infected patient, the dosage of an AAC is normally about 0.001 to 1000 mg/kg/day. In one embodiment the patient with a bacterial infection is treated at an AAC dose in the range of about 1 mg/kg to about 150 mg/kg, typically about 5 mg/kg to about 150 mg/kg, more specifically, 25 mg/kg to 125 mg/kg, 50 mg/kg to 125 mg/kg, even more specifically at about 50 mg/kg to 100 mg/kg. The AAC may be given daily (e.g., a single dose of 5 to 50 mg/kg/day) or less frequently (e.g., a single dose of 5, 10, 25 or 50 mg/kg/week). One dose may be split over 2 days, for example, 25 mg/kg on one day and 25 mg/kg the next day. The patient can be administered a dose once every 3 days (q3D), once a week to every other week (qOW), for a duration of 1-8 weeks. In one embodiment, the patient is administered an AAC of the invention via IV once a week for 2-6 weeks with standard of care (SOC) to treat the bacterial infection such as a staph A infection. Treatment length would be dictated by the condition of the patient or the extent of the infection, e.g. a duration of 2 weeks for uncomplicated bacteremia, or 6 weeks for bacteremia with endocarditis.

In one embodiment, an AAC administered at an initial dose of 2.5 to 100 mg/kg for one to seven consecutive days, followed by a maintenance dose of 0.005 to 10 mg/kg once every one to seven days for one month.

Route of administration. For treating the bacterial infections, the AACs of the invention can be administered at any of the preceding dosages intravenously (i.v.) or subcutaneously. In one embodiment, the rF1-AAC is administered intravenously. In a specific embodiment, the rF1-AAC is administered via i.v., wherein the rF1 antibody is one selected from the group of Abs with amino acid sequences as disclosed under SDR and rF1Abs and Tables 4A and 4B.

Combination therapy. An AAC may be administered in conjunction with one or more additional, e.g. second, therapeutic or prophylactic agents as appropriate as determined by the physician treating the patient.

In one embodiment, the second antibiotic administered in combination with the antibody-antibiotic conjugate compound of the invention is selected from the structural classes: (i) aminoglycosides; (ii) beta-lactams; (iii) macrolides/cyclic peptides; (iv) tetracyclines; (v) fluoroquinolines/fluoroquinolones; (vi) and oxazolidinones. See: Shaw, K. and Barbachyn, M. (2011) Ann. N.Y. Acad. Sci. 1241:48-70; Sutcliffe, J. (2011) Ann. N.Y. Acad. Sci. 1241:122-152.

In one embodiment, the second antibiotic administered in combination with the antibody-antibiotic conjugate compound of the invention is selected from clindamycin, novobiocin, retapamulin, daptomycin, GSK-2140944, CG-400549, sitafloxacin, teicoplanin, triclosan, napthyridone, radezolid, doxorubicin, ampicillin, vancomycin, imipenem, doripenem, gemcitabine, dalbavancin, and azithromycin.

Additional examples of these additional therapeutic or prophylactic agents are anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., azithromycin, clarithromycin, erythromycin, gatifloxacin, levofloxacin, amoxicillin, metronidazole, penicillin G, penicillin V, methicillin, oxacillin, cloxacillin, dicloxacillin, nafcillin, ampicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, moxifloxacin, gemifloxacin, sitafloxacin, daptomycin, garenoxacin, ramoplanin, faropenem, polymyxin, tigecycline, AZD2563, or trimethoprim), antibacterial antibodies including antibodies to the same or different antigen from the AAC targeted Ag, platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). In one embodiment the AAC of the invention is administered in combination with standard of care (SOC) for S. aureus (including methicillin-resistant and methicillin-sensitive strains). MSSA is usually typically treated with nafcillin or oxacillin and MRSA is typically treated with vancomycin or cefazolin.

These additional agents may be administered within 14 days, 7 days, 1 day, 12 hours, or 1 hour of administration of an AAC, or simultaneously therewith. The additional therapeutic agents may be present in the same or different pharmaceutical compositions as an AAC. When present in different pharmaceutical compositions, different routes of administration may be used. For example, an AAC may be administered intravenous or subcutaneously, while a second agent may be administered orally.

### Pharmaceutical Formulations

The present invention also provides pharmaceutical compositions containing the rF1-AAC, and to methods of treating a bacterial infection using the pharmaceutical compositions containing AAC. Such compositions may further comprise suitable excipients, such as pharmaceutically acceptable excipients (carriers) including buffers, acids, bases, sugars, diluents, glidants, preservatives and the like, which are well known in the art and are described herein. The present methods and compositions may be used alone or in combinations with other conventions methods and/or agents for treating infectious diseases. In some embodiments, a pharmaceutical formulation comprises 1) a rF1-AAC of the invention, and 2) a pharmaceutically acceptable carrier. In some embodiments, a pharmaceutical formulation comprises 1) an AAC of the invention and optionally, 2) at least one additional therapeutic agent.

Pharmaceutical formulations comprising an AAC of the invention are prepared for storage by mixing the AAC having the desired degree of purity with optional physiologically acceptable carriers, excipients or stabilizers (Remington's Pharmaceutical Sciences 16th edition, Osol, A. Ed. (1980)) in the form of aqueous solutions or lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride); phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutical formulations to be used for in vivo administration are generally sterile, readily accomplished by filtration through sterile filtration membranes.

Active ingredients may also be entrapped in microcapsule prepared, for example, by co-acervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or AAC of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies or AAC remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

An AAC may be formulated in any suitable form for delivery to a target cell/tissue. For example, AACs may be formulated as liposomes, a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., (1985) Proc. Natl. Acad. Sci. USA 82:3688; Hwang et al., (1980) Proc. Natl Acad. Sci. USA 77:4030; U.S. Pat. No. 4,485,045; U.S. Pat. No. 4,544,545; WO 97/38731; U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Materials and Methods

### Bacterial Strains and Culture:

All experiments were done with MRSA-USA300 NRS384 obtained from NARSA (http://www.narsa.net/control/member/repositories) unless noted otherwise.

Bacteria were grown on tryptic soy agar plates supplemented with 5% sheep blood (TSA plates) for 18 h at 37° C. For liquid cultures, single colonies from TSA plates were inoculated into tryptic soy broth (TSB) and incubated at 37° C. while shaking at 200 rpm for 18 h; 100 fold dilutions of these cultures in fresh TSB were further subcultured for various times.

### MIC Determinations for Extracellular Bacteria

The MIC for extracellular bacteria was determined by preparing serial 2-fold dilutions of the antibiotic in Tryptic Soy Broth. Dilutions of the antibiotic were made in quadruplicate in 96 well culture dishes. MRSA (NRS384 strain of USA300) was taken from an exponentially growing culture and diluted to 1×10⁴ CFU/mL. The bacteria was cultured in the presence of antibiotic for 18-24 hours with shaking at 37° C. and bacterial growth was determined by reading the Optical Density (OD) at 630 nM. The MIC was determined to be the dose of antibiotic that inhibited bacterial growth by >90%.

### MIC Determinations for Intracellular Bacteria

Intracellular MIC was determined on bacteria that were sequestered inside mouse peritoneal macrophages (see below for generation of murine peritoneal macrophages). Macrophages were plated in 24 well culture dishes at a density of 4×10⁵ cells/mL and infected with MRSA at a ratio of 10-20 bacteria per macrophage. Macrophage cultures were maintained in growth media supplemented with 50 ug/mL of gentamycin (an antibiotic that is active only on extracellular bacteria) to inhibit the growth of extracellular bacteria and test antibiotics were added to the growth media 1 day after infection. The survival of intracellular bacteria was assessed 24 hours after addition of the antibiotics. Macrophages were lysed with Hanks Buffered Saline Solution supplemented with 0.1% Bovine Serum Albumin and 0.1% Triton-X, and serial dilutions of the lysate were made in Phosphate Buffered Saline solution containing 0.05% Tween-20. The number of surviving intracellular bacteria was determined by plating on Tryptic Soy Agar plates with 5% defibrinated sheep blood.

### Bacterial Cell Wall Preparations (CWP), Immunoblotting, and ELISA

CWP were generated by incubating 40 mg of pelleted S. aureus or S. epidermidis per mL of 10 mM Tris-HCl (pH 7.4) supplemented with 30% raffinose, 100 μg/ml of lysostaphin (Cell Sciences, Canton, Mass.), and EDTA-free protease inhibitor cocktail (Roche, Pleasanton, Calif.), for 30 min at 37° C. The lysates were centrifuged at 11,600×g for 5 min, and the supernatants containing cell wall components were collected. For immunoprecipitation, CWP were diluted 4 times in NP-40 buffer (120 mM NaCl, 50 mM Tris-HCl pH 8.0, 1% NP-40, complete protease inhibitor cocktail (Roche) and 2 mM dithiothreitol) containing 1 μg/mL of indicated primary antibodies and incubated for 2 h at 4° C., followed by a 1 h incubation with Protein A/G agarose (Thermo, Waltham, Mass.). Whole cell lysates (WCL) were generated by a 30 min incubation at 37° C. in 20 mM Tris-HCl (pH 7.4), 150 mM NaCl, 100 μg/ml of lysostaphin, 1% Triton-X100 (Thermo) and EDTA-free protease inhibitor cocktail. For immunoblot analysis, proteins were separated on a 4-12% Tris-glycine gel, and transferred to a nitrocellulose membrane (Invitrogen, Carlsbad, Calif.), followed by blotting with indicated primary antibodies (1 μg/mL). Antibodies used are listed in Table 1. Lectin studies were performed by immunoprecipitating filtered (0.2 micron) overnight culture supernatants with concanavalin A (ConA)- or sWGA-agarose beads (Vector Labs, Burlingame, Calif.) supplemented with 0.1 mM CaCl₂ and 0.01 mM MnCl₂.

ELISA experiments were performed using standard protocols. Briefly, plates which were pre-coated with CWP were reacted with human IgG preparations, ie. purified human IgG (Sigma), intravenous immunoglobulin Gammagard Liquid (Baxter, Westlake Village, Calif.), pooled serum from healthy donors or from MRSA patients (both generated in-house). The concentrations of anti-staphylococcal IgG present in the serum or purified IgG were calculated by using a calibration curve that was generated with known concentrations of mAb 28.9.9 against peptidoglycan.

Treatment of Bacteria with Human Neutrophil Proteases or Lysosomal Extracts from Human Neutrophils and Cultured Cells

Lysosomal extracts were isolated from human neutrophils, THP-1 cells, and RAW cells, using a Lysosome Enrichment kit (Thermo). A total of 5×10⁷ cells was used to obtain 300 to 500 microgram of total proteins in the lysosomes. Protease inhibitors were omitted from all steps to maintain protease activity in the lysosomes. The plasma membranes of the cells were disrupted by 30 strokes using a dounce homogenizer (Wheaton, Millville, N.J.). The homogenate was centrifuged at 500×g for 5 min to obtain postnuclear supernatant, which was loaded onto the top of a gradient of 8%, 20%, 23%, 27% and 30% (from top to bottom) of iodixanol. After ultracentrifugation at 145,000×g for 2 h at 4° C., we obtained the lysosomes layered between 8% and 20% iodixanol. This lysosomal fraction was diluted into PBS and pelleted by centrifugation at 18,000×g for 30 min at 4° C. The lysosomal pellets were washed with PBS and lysed in 2% CHAPS with Tris-buffered saline to obtain lysosomal extracts.

To analyze the cleavage of SDR proteins by host proteases, S. aureus bacteria were treated with 50 nM of purified human neutrophil serine proteases or 0.1 mg/ml of neutrophil lysosomal extracts in 50 mM Tris (pH 8.0) with 150 mM NaCl and 2 mM CaCl₂; or with 0.1 mg/ml of RAW or THP-1 lysosomal extracts in 50 mM NaCitrate with 100 mM NaCl and 2 mM DTT (pH 5.5). Cathepsin G inhibitor (Calbiochem, Billerica, Mass.) was added at 100 □g/ml. These mixtures were incubated at 37° C. for 30 minutes when using purified proteases or for 1 h when using lysosomal lysates, and centrifuged to pellet bacteria. The supernatants were analyzed by immunoblotting to detect cleavage products. In some experiments, cell wall preparations were obtained from the remaining bacterial pellets and also analyzed by immunoblotting.

### EXAMPLES

### Example 1 Intracellular MRSA are Protected from Conventional Antibiotics

To confirm the hypothesis that mammalian cells provide a protective niche for S. aureus in the presence of antibiotic therapy, the efficacy was compared of three major antibiotics that are currently used as standard of care (SOC) for invasive MRSA infections (vancomycin, daptomycin and linezolid) against extracellular planktonic bacteria versus bacteria sequestered inside murine macrophages (Table 1).

For extracellular bacteria, MRSA was cultured overnight in Tryptic Soy Broth, and the MIC was determined to be the minimum antibiotic dose that prevented growth. For intracellular bacteria, murine peritoneal macrophages were infected with MRSA and cultured in the presence of gentamycin to kill extracellular bacteria. Test antibiotics were added to the culture medium one day post infection, and the total number of surviving intracellular bacteria was determined 24 hours later. The expected serum concentrations for clinically relevant antibiotics was reported in Antimicrobial Agents, Andre Bryskier. ASM Press, Washington D.C. (2005).

TABLE 1
Minimum inhibitory concentrations (MIC) for several antibiotics
on extracellular bacteria grown in liquid culture vs. intracellular
bacteria sequestered inside murine macrophages.

| | Extracellular | Intracellular | |
|---|---|---|---|
| | MRSA | MRSA | Serum Cmax |
| Antibiotics (Abx) | MIC (μg/mL) | MIC (μg/mL) | (μg/mL) |
| Vancomycin | 1 | >100 | 50 |
| Daptomycin | 4 | >100 | 60 |
| Linezolid | 0.3 | >20 | 20 |
| Rifampicin | 0.004 | 50 | 20 |

This analysis with a highly virulent community-acquired MRSA strain USA300 revealed that although extracellular MRSA is highly susceptible to growth inhibition by low concentrations of vancomycin, daptomycin, and linezolid in liquid culture, all three antibiotics failed to kill the same strain of MRSA sequestered inside macrophages exposed to clinically achievable concentrations of the antibiotics. Even rifampicin, thought to be relatively effective at eliminating intracellular pathogens (Vandenbroek, P. V. (1989) Antimicrobial Drugs, Microorganisms, and Phagocytes. Reviews of Infectious Diseases 11, 213-245), required a 6,000-fold higher dose to eliminate intracellular MRSA compared to the dose required to inhibit growth (MIC) of planktonic bacteria (Table 1), consistent with other studies showing that the majority of existing antibiotics are inefficient at killing intracellular S. aureus both in vitro and in vivo (Sandberg, A., Hessler, J. H., Skov, R. L., Blom, J. & Frimodt-Moller, N. (2009) “Intracellular activity of antibiotics against Staphylococcus aureus in a mouse peritonitis model” Antimicrob Agents Chemother 53, 1874-1883).

### Example 2 Dissemination of Infection with Intracellular MRSA

These experiments compared the virulence of intracellular bacteria versus an equivalent dose of free-living planktonic bacteria, and determined whether the intracellular bacteria are able to establish infection in the presence of vancomycin in vivo. Four cohorts of mice were infected by intravenous injection with roughly equivalent doses of S. aureus viable free bacteria (2.9×10⁶) taken directly from broth culture or intracellular bacteria (1.8×10⁶) sequestered inside host macrophages and neutrophils that were generated by peritoneal infection of donor mice (FIG. 1A) and selected groups were treated with vancomycin immediately after infection and then once per day. Mice were examined 4 days after infection for bacterial colonization in the kidney, an organ that is consistently colonized by S. aureus in mice²³. In three independent experiments, equivalent or higher bacterial burdens in the kidneys of mice infected with intracellular bacteria compared to those infected with an equivalent dose of planktonic bacteria was observed (FIG. 1B). Surprisingly, it was found that infection with intracellular bacteria resulted in more consistent colonization of the brain, an organ that is not efficiently colonized following infection with planktonic bacteria in this model (FIG. 1C). Furthermore, intracellular bacteria, but not planktonic bacteria, were able to establish infection in the face of vancomycin therapy in this model (FIG. 1B, FIG. 1C)

Further analyses in vitro addressed more quantitatively the extent to which intracellular survival facilitates antibiotic evasion. To this end, MG63 osteoblasts were infected with either planktonic MRSA or intracellular MRSA, in the presence of vancomycin.

Infection of osteoblasts or HBMEC. MG63 cell line was obtained from ATCC (CRL-1427) and maintained in RPMI 1640 tissue culture media supplemented with 10 mM Hepes and 10% Fetal Calf Serum (RPMI-10). HBMEC cells (Catalog #1000) and ECM media (catalog#1001) were obtained from SciencCell Research Labs (Carlsbad, Calif.). Cells were plated in 24 well tissue culture plates and cultured to obtain a confluent layer. On the day of the experiment, the cells were washed once in RPMI (without supplements). MRSA or infected peritoneal cells were diluted in complete RPMI-10 and vancomycin was added at 5 ug/mL immediately prior to infection. Peritoneal cells were added to the osteoblasts at 1×106 peritoneal cells/mL. A sample of the cells was lysed with 0.1% triton-x to determine the actual concentration of live intracellular bacteria at the time of infection. The actual titer for all infections was determined by plating serial dilutions of the bacteria on Tryptic Soy Agar with 5% defibrinated sheep blood.

MRSA (free bacteria) was seeded in media, media+vancomycin, or media+vancomycin and plated on a monolayer of MG63 osteoblasts (FIG. 1E) or Human Brain Microvascular Endothelial Cells (HBMEC, FIG. 1F). Plates were centrifuged to promote contact of the bacteria with the monolayer. At each time point, the culture supernatant was collected to recover extracellular bacteria or adherent cells were lysed to release intracellular bacteria.

Planktonic bacteria exposed to vancomycin alone were efficiently killed. Surviving bacteria were not recovered after one day in culture (FIG. 1D). When a similar number of planktonic bacteria were plated on MG63 osteoblasts, a small number of surviving bacteria (approximately 0.06% of input) associated with the MG63 cells one day after infection, which had been protected from vancomycin by invasion of the osteoblasts, was recovered.

MRSA that were sequestered inside peritoneal cells showed a dramatic increase in both survival and efficiency of infection in the presence of vancomycin. About 15% of intracellular MRSA in the leukocytes survived under identical conditions where vancomycin had sterilized the cultures of planktonic bacteria. Intracellular bacteria also were better able to infect the monolayer of MG63 osteoblasts in the presence of vancomycin, resulting in a doubling of the bacteria recovered one day after exposure to vancomycin (FIG. 1D). Moreover, intracellular S. aureus were able to increase by almost 10-fold over a 24 hour period in MG63 cells (FIG. 1E), primary human brain endothelial cells (FIG. 1F), and A549 bronchial epithelial cells (not shown) under constant exposure to a concentration of vancomycin that killed free living bacteria. Although protected from antibiotic killing, bacterial growth did not occur in cultures of infected peritoneal macrophages and neutrophils (not shown). Together these data support that intracellular reservoirs of MRSA in myeloid cells can promote dissemination of infection to new sites, even in the presence of active antibiotic treatment, and intracellular growth can occur in endothelial and epithelial cells, even under conditions of constant antibiotic therapy.

### Example 3 Generation of Anti-SDR and Other Antibodies

For generation of mAb rF1, CD19+CD3-CD27+IgD-IgA-memory B cells were isolated from peripheral blood of an MRSA-infected donor using a FACSAria cell sorter (BD, San Jose, Calif.). Before viral transduction with B-cell lymphoma (Bcl)-xL and Bcl-6 genes, the memory cells were activated on CD40L-expressing mouse L fibroblasts in the presence of interleukin-21, as described previously in Kwakkenbos M J, et al. (2010) Nat Med 16: 123-128. Transduced B cells were maintained in the same culture system. The use of donor blood was approved by the institutional committee. Monoclonal antibody (mAb) rF1 was selected from culture supernatants by reactivity with lysates of MSSA strain Newman by ELISA; positive wells were subcloned and re-tested by ELISA twice. Recombinant rF1 was generated by cloning the heavy and light chain variable regions with human IgG1 kappa constant regions using pcDNA3.1 (Invitrogen) and transfection into 293T cells (ATCC). Purified IgG was obtained from culture supernatants using protein A-coupled SEPHAROSE® (Invitrogen). The generation of mAb rF1 and its variants are described in U.S. Pat. No. 8,617,556 (Beaumont et al.) and Hazenbos et al. (2103) PLOS Pathogens 9(10): 1-18, incorporated by reference herein in their entirety.

The human IgG1 mAbs SD2, SD3 and SD4 (all against glycosylated SDR proteins) and 4675 (human IgG1 anti-ClfA), were cloned from peripheral B cells from patients post S. aureus infection using the Symplex™ technology which conserves the cognate pairing of antibody heavy and light chains [34]. Both plasma and memory B-cells were used as genetic source for the recombinant full length IgG repertoires (manuscript in preparation). Individual antibody clones were expressed by transfection of mammalian cells [35]. Supernatants containing full length IgG1 antibodies were harvested after seven days and used to screen for antigen binding by ELISA. Antibodies 4675, SD2, SD3 and SD4 were positive for binding to cell wall preparations from USA300 or Newman S. aureus strains. Antibodies were subsequently produced in 200-ml transient transfections and purified with Protein A chromatography (MabSelect SuRe, GE Life Sciences, Piscataway, N.J.) for further testing. Isolation and usage of these antibodies were approved by the regional ethical review board. rF1 variants were generated.

Mouse mAb against ClfA (9E10), ClfB, (10D2), SdrD (17H4), IsdA (2D3) and non-modified SDR proteins (9G4) were generated by immunizing mice with the respective recombinant proteins, which were purified after expression in E. coli, using standard protocols; hybridoma supernatants were purified by protein A affinity chromatography. Rabbit mAb 28.9.9 was generated by immunizing rabbits with peptidoglycan (PGN)-derived peptide CKKGGG-(L-Ala)-(D-gamma-Glu)-(L-Lys)-(D-Ala)-D-Ala) followed by cloning of the IgG.

### Example 4 Characterization of a Highly Opsonic Monoclonal Antibody (rF1) Isolated from an MRSA Infected Donor

Several S. aureus-reactive monoclonal antibodies (mAb) from memory B cells from peripheral blood of MRSA-infected donors were isolated as described above. When characterizing these antibodies, one IgG1 mAb (hereafter referred to as rF1) was identified with broad reactivity to a panel of S. aureus strains that induced robust opsonophagocytic killing (OPK) by human polymorphonuclear leukocytes (PMN).

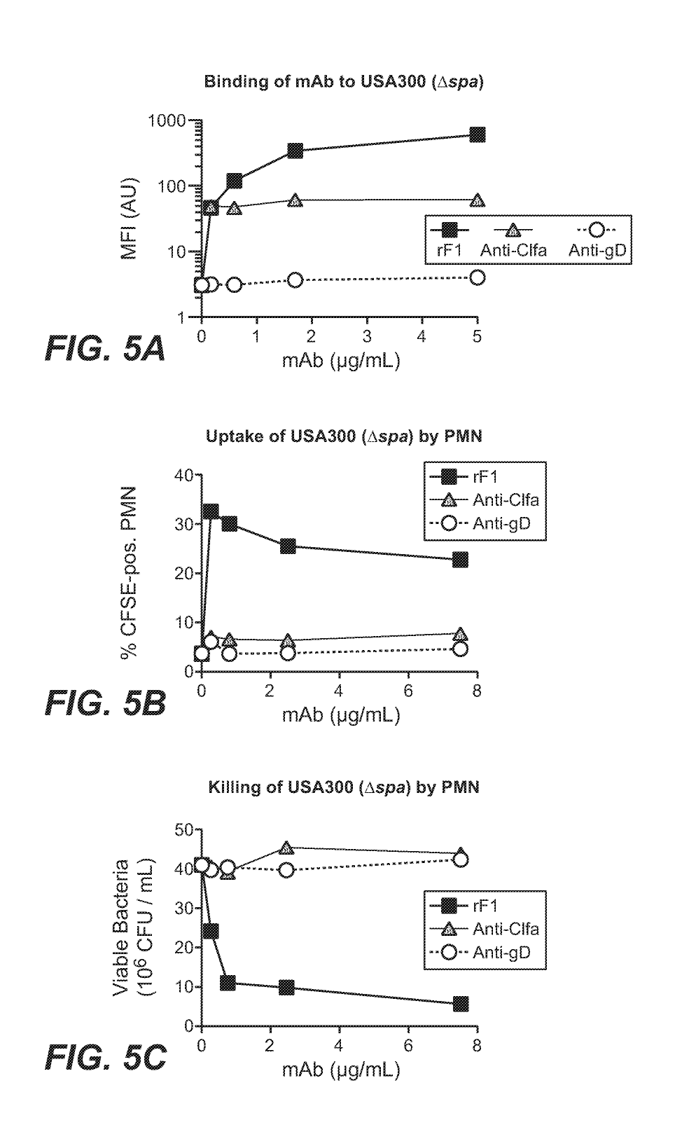

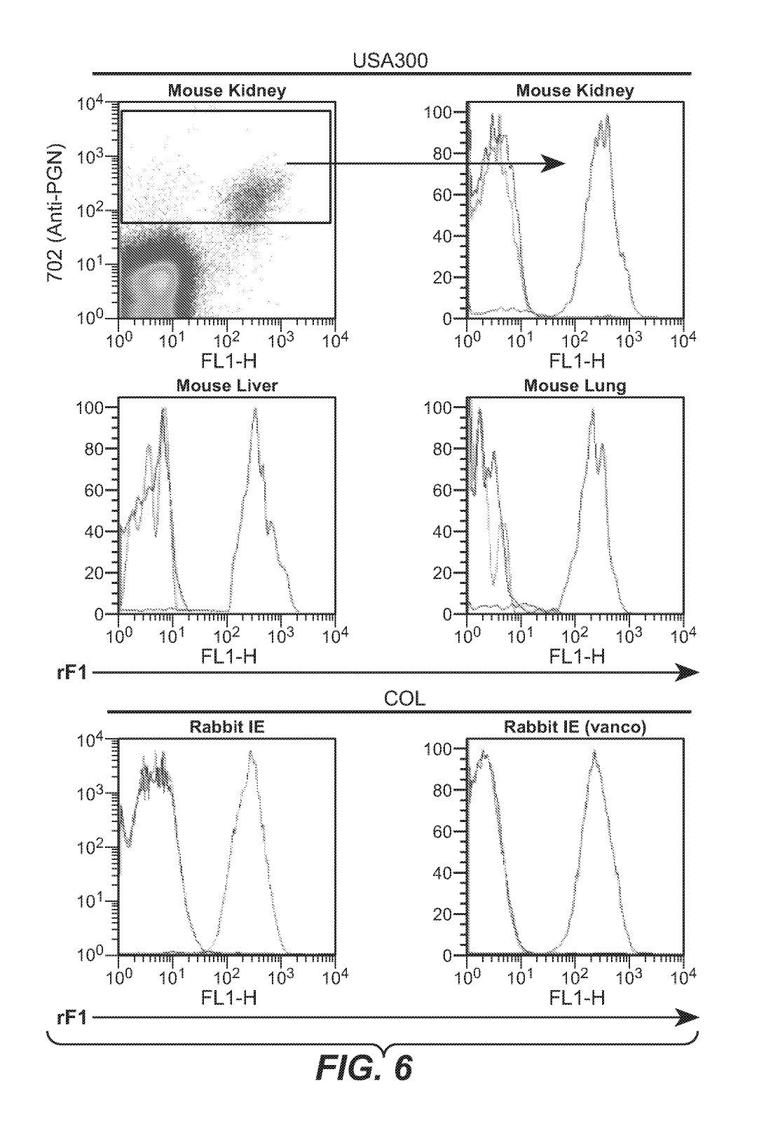

Maximum binding of mAb rF1 to bacteria from clinical MRSA strain USA300 was approximately 10 fold higher than that of an isotype-matched anti-ClfA mAb (FIG. 5A). Consistent with increased binding, opsonization with rF1 resulted in increased uptake (FIG. 5B) and killing (FIG. 5C) of USA300 by PMN. In contrast, preopsonization with human anti-ClfA had no effect on bacterial viability (FIG. 5C). The rF1 antibody did not affect viability of USA300 in the absence of PMN. Thus, rF1 is a mAb with the capacity to bind MRSA and induce potent killing of MRSA by PMN.

### Example 5 Binding of rF1 to Staphylococcus Strains

FACS Analysis of rF1 Binding to Whole Bacteria from Culture or Infected Tissues

Whole bacteria were harvested from TSA plates or TSB cultures and washed with HBSS without phenol red supplemented with 0.1% IgG free BSA (Sigma) and 10 mM Hepes, pH 7.4 (HB buffer) Bacteria (20×10⁸ CFU/mL) were incubated with 300 μg/mL of rabbit IgG (Sigma) in HB buffer for 1 h at room temperature (RT) to block nonspecific IgG binding. Bacteria were stained with 2 μg/mL of primary antibodies, including rF1 or isotype control IgG1 mAb gD:5237 (Nakamura G R, et al. (1993) J Virol 67: 6179-6191), and next with fluorescent anti-human IgG secondary antibodies (Jackson Immunoresearch, West Grove, Pa.). The bacteria were washed and analyzed by FACSCalibur® (BD).

For antibody staining of bacteria from infected mouse tissues, 6-8 weeks old female C57Bl/6 mice (Charles River, Wilmington, Mass.) were injected intravenously with 108 CFU of logphase-grown USA300 in PBS. Mouse organs were harvested two days after infection. Rabbit infective endocarditis (IE) was established as described in Tattevin P, et al. (2010) Antimicrobial agents and chemotherapy 54: 610-613. Rabbits were injected intravenously with 5×107 CFU of stationary-phase grown MRSA strain COL, and heart vegetations were harvested eighteen hours later. Treatment with 30 mg/kg of vancomycin was given intravenously b.i.d. 18 h after infection with 7×107 CFU stationary-phase COL.

To lyse mouse or rabbit cells, tissues were homogenized in M tubes (Miltenyi, Auburn, Calif.) using a gentleMACS® cell dissociator (Miltenyi), followed by incubation for 10 min at RT in PBS containing 0.1% Triton-X100 (Thermo), 10 □g/mL of DNAseI (Roche) and Complete Mini protease inhibitor cocktail (Roche). The suspensions were passed through a 40 micron filter (BD) and bacteria were stained with mAbs as described above. Bacteria were differentiated from mouse organ debris by double staining with 20 □g/mL mouse mAb 702 anti-S. aureus peptidoglycan (abcam, Cambridge, Mass.) and a fluorochrome-labeled anti-mouse IgG secondary antibody (Jackson Immunoresearch). During flow cytometry analysis, bacteria were gated for positive staining with mAb 702 from double fluorescence plots. All animal experiments were approved by the Institutional Review Boards of Genentech and the University of California, San Francisco.

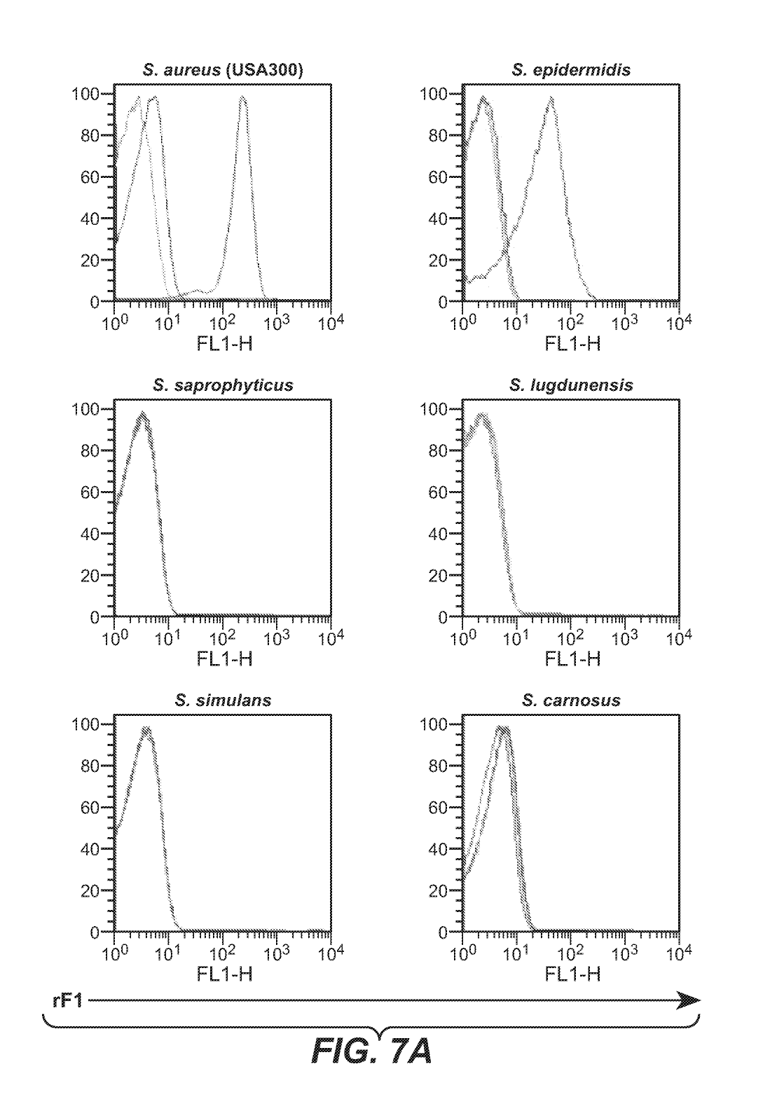

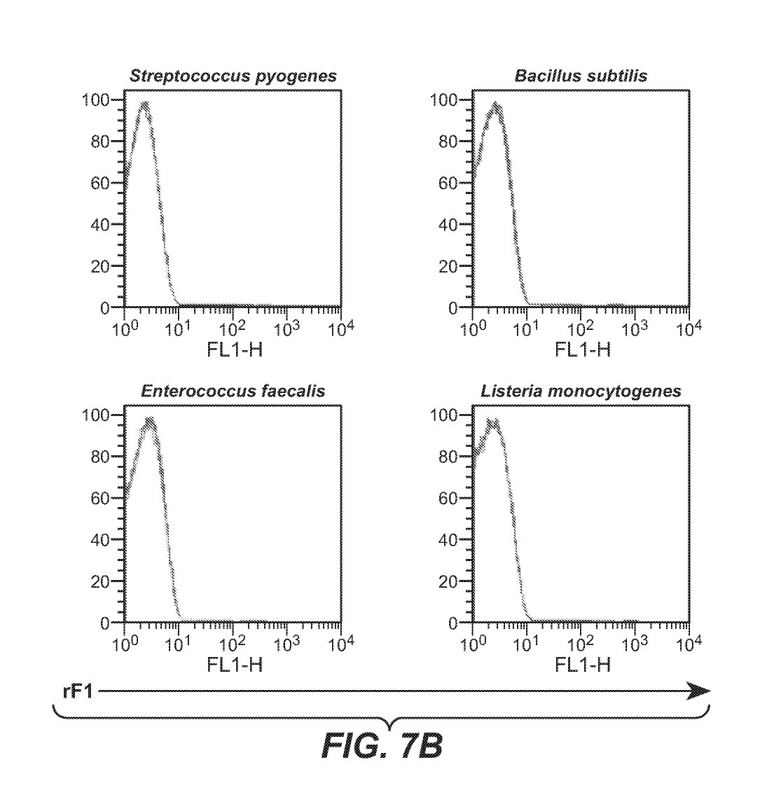

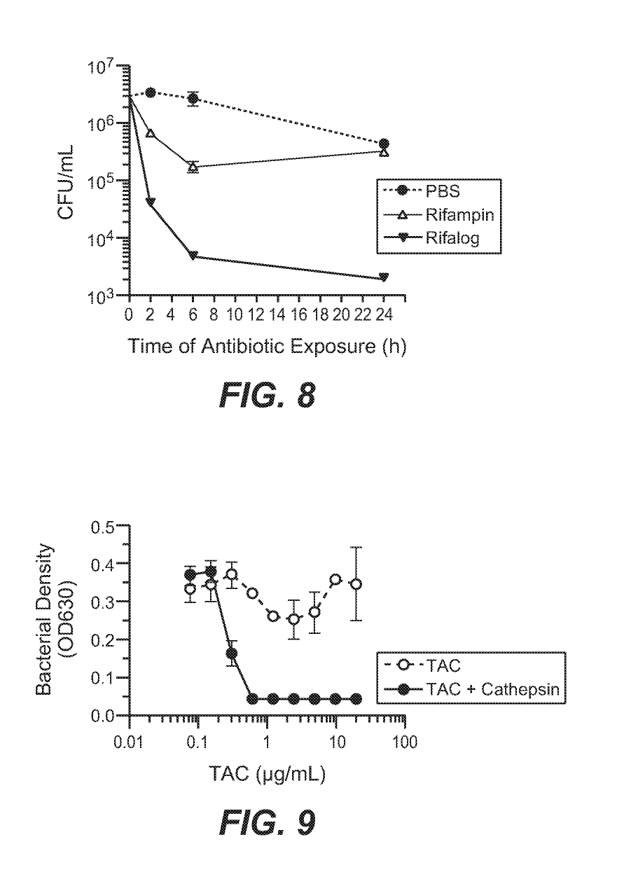

Flow cytometry (FCM) analysis showed potent binding activity of rF1 to all 15 S. aureus strains tested (FIG. 7). These strains were broadly distributed across the S. aureus phylogeny [8]. As expression levels of bacterial cell surface antigens might differ between in vitro and in vivo growth, we also tested the ability of rF1 to recognize USA300 isolated from various mouse tissues after systemic infection. The rF1 mAb strongly bound to USA300 derived from infected mouse kidneys, livers and lungs (FIG. 6). The binding rF1 to USA300 from mouse kidneys was sustained until at least 8 days after infection (not shown), suggesting robust long-term expression of the rF1 epitope during infection. In addition, rF1 strongly bound to MRSA COL bacteria from heart vegetations in a rabbit model of infectious endocarditis. Treatment with vancomycin did not affect the reactivity of rF1 with MRSA (FIG. 6). Thus, the antigen recognized by rF1 is conserved across various strains and stably expressed in various growth and infection conditions.

Given the ubiquitous nature of rF1-reactivity across all S. aureus strains, experiments were performed to see if such reactivity is extended to other gram-positive bacteria. Notably, rF1 binding was detectable only for the coagulase-negative human pathogen S. epidermidis (FIG. 7). The rF1 mAb did not bind to any other staphylococcal species tested, including S. saprophyticus, S. lugdunensis, S. simulans and S. carnosus, or other Gram-positive species such as Streptococcus pyogenes, Bacillus subtilis, Enterococcus faecalis, and Listeria monocytogenes (FIG. 7). Thus, rF1 is a human antibody that binds to stably-expressed surface antigen(s) on human-adapted staphylococcal pathogens and promotes bacterial killing by human PMNs.

### Example 6 Amino Acid Modifications of rF1 Antibodies

In summary, the VH region of each of the rF1 Abs were cloned out and linked to human H chain gammal constant region and the VL linked to kappa constant region to express the Abs as IgG1. Wild-type sequences were altered at certain positions to improve the antibody stability while maintaining antigen binding as described below. Cysteine engineered Abs (ThioMabs, also referred to as THIOMAB™) were then generated.

i. Generating Stability Variants

The rF1 Abs were engineered to improve certain properties (to avoid deamidation, aspartic acid isomerization, oxidation or N-linked glycosylation) and tested for retention of antigen binding as well as chemical stability after amino acid replacements. The amino acid alterations made were as described in U.S. Pat. No. 8,617,556.

iii. Generating Cys Engineered Mutants (ThioMabs)

Full length ThioMabs were produced by introducing a Cysteine into the H chain (in CH1) or the L chain (Cκ) at a predetermined position as previously taught, e.g., at V205 in the kappa Constant region of the L chain and position A118 in the human Gamma 1 H chain (amino acid position numbers according to Eu convention) to allow conjugation of the antibody to a linker-antibiotic intermediate. H and L chains are then cloned into separate plasmids and the H and L encoding plasmids co-transfected into 293 cells where they are expressed and assembled into intact Abs. Both H and L chains can also be cloned into the same expression plasmid. IgG1 having 2 engineered Cys, one in each of H chains; or 2 engineered Cys, one in each of the L chains; or a combination of an engineered Cys in each of the H and L chains (HC LC Cys) leading to 4 engineered Cys per antibody tetramer, were generated by expressing the desired combination of cys mutant chains and wild type chains.

### Example 7 Piperidyl Benzoxazino Rifamycin (pipBOR) 5

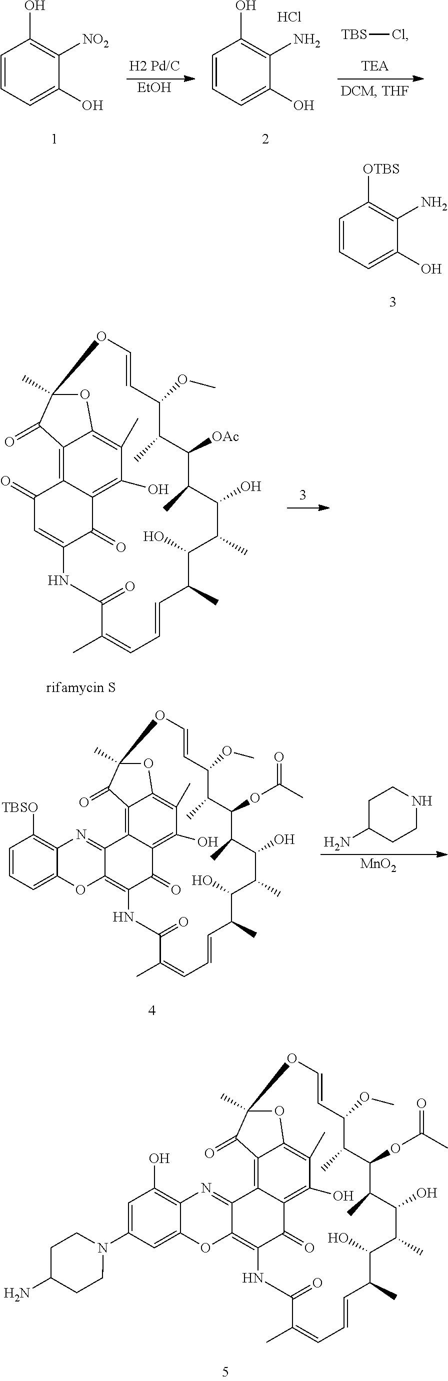

2-Nitrobenzene-1,3-diol 1 was hydrogenated under hydrogen gas with palladium/carbon catalyst in ethanol solvent to give 2-aminobenzene-1,3-diol 2, isolated as the hydrochloride salt. Mono-protection of 2 with tert-butyldimethylsilyl chloride and triethylamine in dichloromethane/tetrahydrofuran gave 2-amino-3-(tert-butyldimethylsilyloxy)phenol 3. Rifamycin S (ChemShuttle Inc., Fremont, Calif., U.S. Pat. No. 7,342,011; U.S. Pat. No. 7,271,165; U.S. Pat. No. 7,547,692) was reacted with 3 by oxidative condensation with manganese oxide or oxygen gas in toluene at room temperature to give TBS-protected benzoxazino rifamycin 4. LCMS (ESI): M+H⁺=915.41. Reaction of 4 with piperidin-4-amine and manganese oxide gave piperidyl benzoxazino rifamycin (pipBOR) 5. LCMS (ESI): M+H⁺=899.40

### Example 8 DimethylpipBOR 6

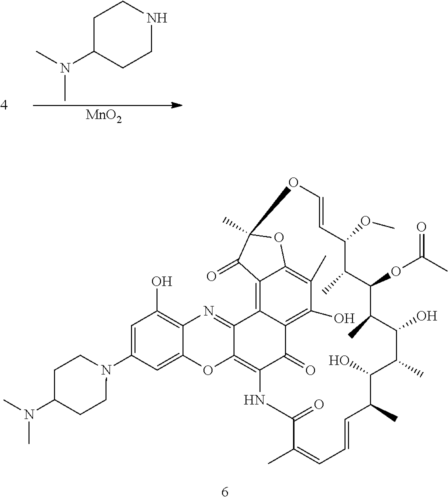

Reaction of N,N-dimethylpiperidin-4-amine with TBS-protected benzoxazino rifamycin 4 gave dimethylpiperidyl benzoxazino rifamycin (dimethylpipBOR) 6

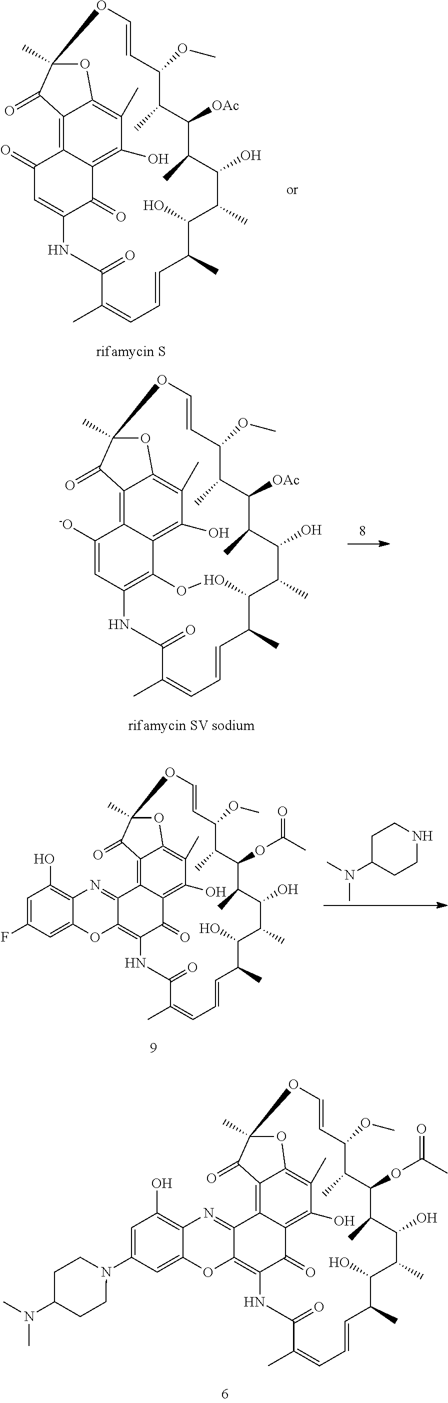

Alternatively, (5-fluoro-2-nitro-1,3-phenylene)bis(oxy)bis(methylene)dibenzene 7 was hydrogenated under hydrogen gas with palladium/carbon catalyst in tetrahydrofuran/methanol solvent to remove the benzyl groups to give 2-amino-5-fluorobenzene-1,3-diol 8. LCMS (ESI): M+H⁺=144.04. Commercially available Rifamycin S or Rifamycin SV sodium salt (ChemShuttle Inc., Fremont, Calif.) was reacted with 2-amino-5-fluorobenzene-1,3-diol 8 by oxidative condensation in air or potassium ferric cyanide in ethyl acetate at 60° C. to give fluorobenzoxazino rifamycin 9. Displacement of fluoride with N,N-dimethylpiperidin-4-amine gave dimethylpipBOR 6. LCMS (ESI): M+H⁺=927.43

### Example 9 (S)—N-(5-(2,5-dioxo-2,5-dihydro-1H-pyrrol-1-yl)pentyl)-N-(1-(4-(hydroxymethyl)phenylamino)-1-oxo-5-ureidopentan-2-yl)cyclobutane-1,1-dicarboxamide 10

### Step 1: Preparation of 1-(5-aminopentyl)-1H-pyrrole-2,5-dione hydrochloride 10a

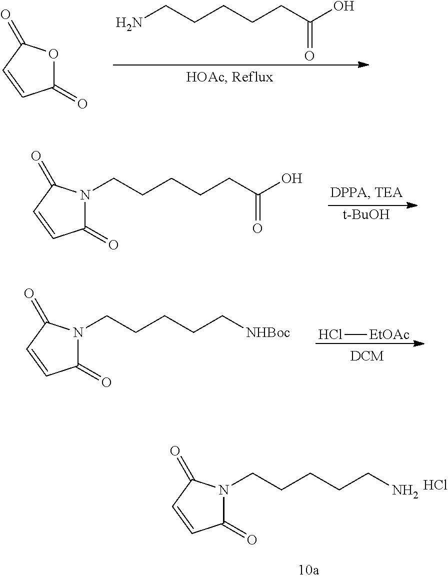

Maleic anhydride, furan-2,5-dione (150 g, 1.53 mol) was added to a stirred solution of 6-aminohexanoic acid (201 g, 1.53 mol) in HOAc (1000 mL). After the mixture was stirred at r.t. for 2 h, it was heated at reflux for 8 h. The organic solvents were removed under reduced pressure and the residue was extracted with EtOAc (500 mL×3), washed with H₂O. The combined organic layers was dried over Na₂SO₄ and concentrated to give the crude product. It was washed with petroleum ether to give 6-(2,5-dioxo-2,5-dihydro-1H-pyrrol-1-yl)hexanoic acid as white solid (250 g, 77.4%). DPPA (130 g, 473 mmol) and TEA (47.9 g, 473 mmol) was added to a solution of 6-(2,5-dioxo-2,5-dihydro-1H-pyrrol-1-yl)hexanoic acid (100 g, 473 mmol) in t-BuOH (200 mL). The mixture was heated at reflux for 8 h under N₂. The mixture was concentrated, and the residue was purified by column chromatography on silica gel (PE:EtOAc=3:1) to give tert-butyl 5-(2,5-dioxo-2,5-dihydro-1H-pyrrol-1-yl)pentylcarbamate (13 g, 10%). To a solution of tert-butyl 5-(2,5-dioxo-2,5-dihydro-1H-pyrrol-1-yl)pentylcarbamate (28 g, 992 mmol) in anhydrous EtOAc (30 mL) was added HCl/EtOAc (50 mL) dropwise. After the mixture was stirred at r.t. for 5 h, it was filtered and the solid was dried to give 1-(5-aminopentyl)-1H-pyrrole-2,5-dione hydrochloride 10a (16 g, 73.7%). ¹H NMR (400 MHz, DMSO-d₆): δ 8.02 (s, 2H), 6.99 (s, 2H), 3.37-3.34 (m, 2H), 2.71-2.64 (m, 2H), 1.56-1.43 (m, 4H), 1.23-1.20 (m, 2H).

### Step 2: Preparation of (S)-1-(1-(4-(hydroxymethyl)phenylamino)-1-oxo-5-ureidopentan-2-ylcarbamoyl)cyclobutanecarboxylic acid 10b

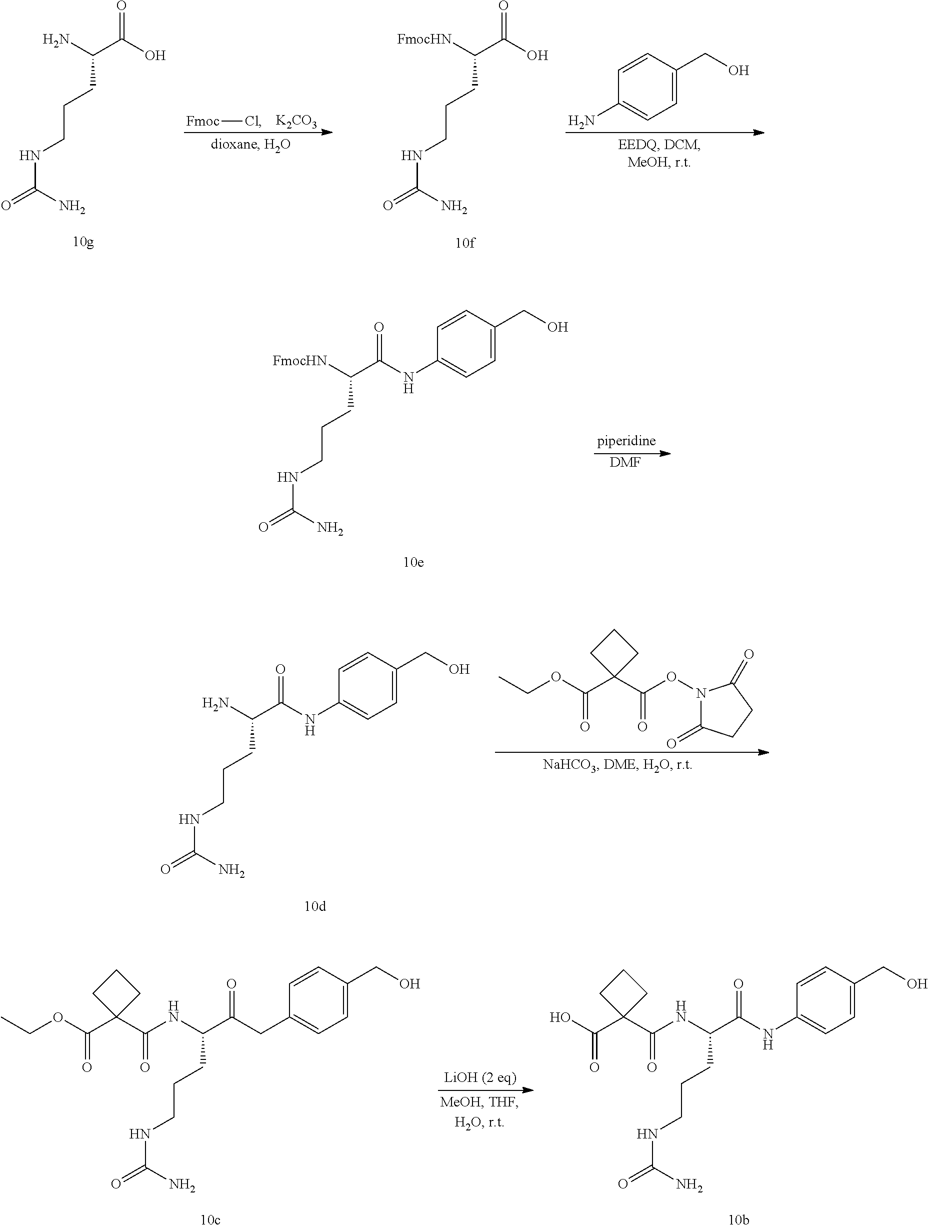

To a mixture of (S)-2-amino-5-ureidopentanoic acid 10 g (17.50 g, 0.10 mol) in a mixture of dioxane and H₂O (50 mL/75 mL) was added K₂CO₃ (34.55 g, 0.25 mol). Fmoc-Cl (30.96 g, 0.12 mol) was added slowly at 0° C. The reaction mixture was warmed to r.t. over 2 h. Organic solvent was removed under reduced pressure, and the water slurry was adjusted to pH=3 with 6 M HCl solution, and extracted with EtOAc (100 mL×3). The organic layer was dried over Na₂SO₄, filtered, and concentrated under reduced pressure to give (S)-2-((((9H-fluoren-9-yl)methoxy)carbonyl)amino)-5-ureidopentanoic acid 10f (38.0 g, 95.6%). 10f is commercially available.

To a solution of 10f (4 g, 10 mmol) in a mixture of DCM and MeOH (100 mL/50 mL) were added (4-aminophenyl)methanol (1.6 g, 13 mmol, 1.3 eq) and 2-Ethoxy-1-ethoxycarbonyl-1,2-dihydroquinoline, EEDQ, Sigma-Aldrich CAS Reg. No. 16357-59-8 (3.2 g, 13 mmol, 1.3 eq). After the mixture was stirred at r.t. for 16 h under N2, it was concentrated to give a brown solid. MTBE (200 mL) was added and it was stirred at 15° C. for 2 h. The solid was collected by filtration, washed with MTBE (50 mL×2) to give (S)-(9H-fluoren-9-yl)methyl (1-((4-(hydroxymethyl)phenyl)amino)-1-oxo-5-ureidopentan-2-yl)carbamate 10e as an orange solid (4.2 g, 84%). LCMS (ESI): m/z 503.0 [M+1].

To a stirred solution of 10e (4.2 g, 8.3 mmol) in dry DMF (20 ml) was added piperidine (1.65 mL, 17 mmol, 2 eq) dropwise at r.t. The mixture was stirred at r.t. for 30 min, and solid precipitate formed. Dry DCM (50 mL) was added, and the mixture became transparent immediately. The mixture was stirred at r.t. for another 30 min, and LCMS showed 10e was consumed. It was concentrated to dryness under reduced pressure (make sure no piperidine remained), and the residue was partitioned between EtOAc and H₂O (50 mL/20 mL). Aqueous phase was washed with EtOAc (50 mL×2) and concentrated to give (S)-2-amino-N-(4-(hydroxymethyl)phenyl)-5-ureidopentanamide 10d as an oily residual (2.2 g, 94%) (contained small amount of DMF).

Commercially available 1,1-cyclobutanedicarboxylic acid, 1,1-diethyl ester (CAS Reg. No. 3779-29-1) was converted by limited saponification with aqueous base to the half acid/ester 1,1-cyclobutanedicarboxylic acid, 1-ethyl ester (CAS Reg No. 54450-84-9) and activation with a coupling reagent such as TBTU (O-(Benzotriazol-1-yl)-N,N,N′,N′-tetramethyluronium tetrafluoroborate, also called: N,N,N′,N′-Tetramethyl-O-(benzotriazol-1-yl)uronium tetrafluoroborate, CAS No. 125700-67-6, Sigma-Aldrich B-2903), and N-hydroxysuccinimide to the NHS ester, 1-(2,5-dioxopyrrolidin-1-yl) 1-ethyl cyclobutane-1,1-dicarboxylate.

To a solution of 1-(2,5-dioxopyrrolidin-1-yl) 1-ethyl cyclobutane-1,1-dicarboxylate (8 g, 29.7 mmol) in DME (50 mL) was added a solution of 10d (6.0 g, 21.4 mmol) and NaHCO3 (7.48 g, 89.0 mmol) in water (30 mL). After the mixture was stirred at r.t. for 16 h, it was concentrated to dryness under reduced pressure and the residue was purified by column chromatography (DCM:MeOH=10:1) to give (S)-ethyl 1-((1-(4-(hydroxymethyl)phenyl)-2-oxo-6-ureidohexan-3-yl)carbamoyl)cyclobutanecarboxylate 10c as white solid (6.4 g, 68.7%). LCMS (ESI): m/z 435.0 [M+1]

To a stirred solution of 10c (6.4 g, 14.7 mmol) in a mixture of THF and MeOH (20 mL/10 mL) was added a solution of LiOH—H₂O (1.2 g, 28.6 mmol) in H₂O (20 mL) at r.t. After the reaction mixture was stirred at r.t. for 16 h, solvent was removed under reduced pressure, the residue obtained was purified by prep-HPLC to give (S)-1-(1-(4-(hydroxymethyl)phenylamino)-1-oxo-5-ureidopentan-2-ylcarbamoyl)cyclobutanecarboxylic acid 10b (3.5 g, yield: 58.5%). LCMS (ESI): m/z 406.9 [M+1]. 1H NMR (400 MHz, Methanol-d4) δ 8.86 (d, J=8.4 Hz, 2H), 8.51 (d, J=8.4 Hz, 2H), 5.88-5.85 (m, 1H), 5.78 (s, 2H), 4.54-4.49 (m, 3H), 4.38-4.32 (m, 1H), 3.86-3.75 (m, 1H), 3.84-3.80 (m, 2H), 3.28-3.21 (m, 1H), 3.30-3.24 (m, 1H), 3.00-2.80 (m, 1H), 2.37-2.28 (m, 2H).

### Step 3: Preparation of S)—N-(5-(2,5-dioxo-2,5-dihydro-1H-pyrrol-1-yl)pentyl)-N-(1-(4-(hydroxymethyl)phenylamino)-1-oxo-5-ureidopentan-2-yl)cyclobutane-1,1-dicarboxamide

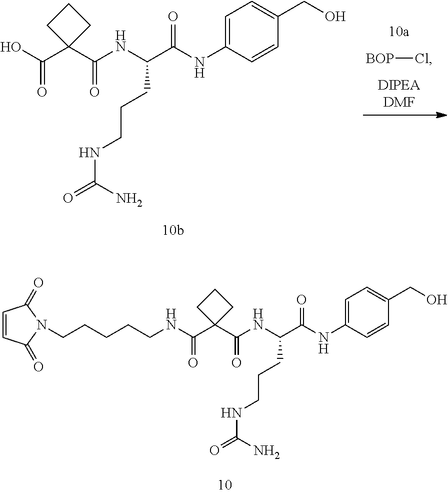

Diisopropylethylamine, DIPEA (1.59 g, 12.3 mmol) and bis(2-oxo-3-oxazolidinyl)phosphinic chloride, BOP-Cl (CAS Reg. No. 68641-49-6, Sigma-Aldrich, 692 mg, 2.71 mmol) was added to a solution of (S)-1-(1-(4-(hydroxymethyl)phenylamino)-1-oxo-5-ureidopentan-2-ylcarbamoyl)cyclobutanecarboxylic acid 10b (1 g, 2.46 mmol) in DMF (10 mL) at 0° C., followed by 1-(5-aminopentyl)-1H-pyrrole-2,5-dione hydrochloride 10a (592 mg, 2.71 mmol). The mixture was stirred at 0° C. for 0.5h. The reaction mixture was quenched with citric acid solution (10 mL), extracted with DCM/MeOH (10:1). The organic layer was dried and concentrated, and the residue was purified by column chromatography on silica gel (DCM:MeOH=10:1) to give to give S)—N-(5-(2,5-dioxo-2,5-dihydro-1H-pyrrol-1-yl)pentyl)-N-(1-(4-(hydroxymethyl)phenylamino)-1-oxo-5-ureidopentan-2-yl)cyclobutane-1,1-dicarboxamide 10 (1.0 g, 71%), also referred to as MC-CBDK-cit-PAB-OH. LCMS (ESI): M+H⁺=571.28. ¹H NMR (400 MHz, DMSO-d₆): δ 10.00 (s, 1H), 7.82-7.77 (m, 2H), 7.53 (d, J=8.4 Hz, 2H), 7.19 (d, J=8.4 Hz, 2H), 6.96 (s, 2H), 5.95 (t, J=6.4 Hz, 1H), 5.39 (s, 2H), 5.08 (t, J=5.6 Hz, 1H), 4.40-4.35 (m, 3H), 4.09 (d, J=4.8 Hz, 1H), 3.01 (d, J=3.2 Hz, 2H), 3.05-2.72 (m, 4H), 2.68-2.58 (m, 3H), 2.40-2.36 (m, 4H), 1.72-1.70 (m, 3H), 1.44-1.42 (m, 1H), 1.40-1.23 (m, 6H), 1.21-1.16 (m, 4H).

### Example 10 (S)—N-(1-(4-(chloromethyl)phenylamino)-1-oxo-5-ureidopentan-2-yl)-N-(5-(2,5-dioxo-2,5-dihydro-1H-pyrrol-1-yl)pentyl)cyclobutane-1,1-dicarboxamide 11

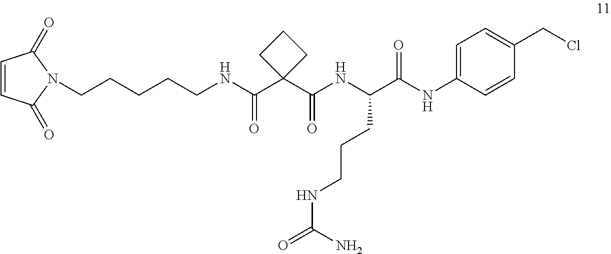 [SMILES: NC(=O)NCCC[C@H](NC(=O)C1(C(=O)NCCCCCN2C(=O)C=CC2=O)CCC1)C(=O)Nc1ccc(CCl)cc1]

A solution of (S)—N-(5-(2,5-dioxo-2,5-dihydro-1H-pyrrol-1-yl)pentyl)-N-(1-(4-(hydroxymethyl)phenylamino)-1-oxo-5-ureidopentan-2-yl)cyclobutane-1,1-dicarboxamide 10 (2.0 g, 3.5 mmol) in N,N-dimethylformamide, DMF or N-methylpyrrolidone, NMP (50 mL) was treated with thionyl chloride, SOCl₂ (1.25 g, 10.5 mmol) in portions dropwise at 0° C. The reaction remained yellow. The reaction was monitored by LC/MS indicating >90% conversion. After the reaction mixture was stirred at 20° C. for 30 min or several hours, it was diluted with water (50 mL) and extracted with EtOAc (50 mL×3). The organic layer was dried, concentrated and purified by flash column (DCM:MeOH=20:1) to form 11, also referred to as MC-CBDK-cit-PAB-Cl as a gray solid. LCMS: (5-95, AB, 1.5 min), 0.696 min, m/z=589.0 [M+1]⁺.

### Example 11 (S)-4-(2-(1-(5-(2,5-dioxo-2,5-dihydro-1H-pyrrol-1-yl)pentylcarbamoyl)cyclobutanecarboxamido)-5-ureidopentanamido)benzyl 4-nitrophenyl carbonate 12

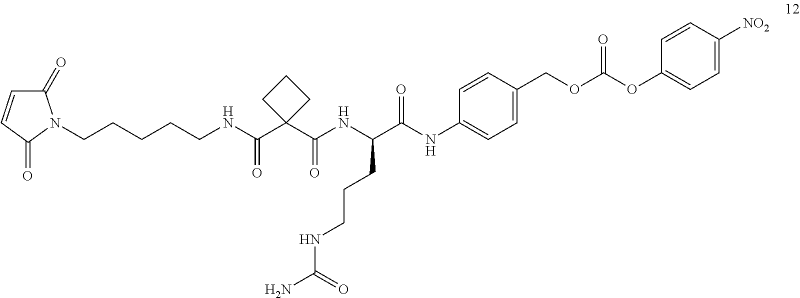 [SMILES: NC(=O)NCCC[C@@H](NC(=O)C1(C(=O)NCCCCCN2C(=O)C=CC2=O)CCC1)C(=O)Nc1ccc(COC(=O)Oc2ccc([N+](=O)[O-])cc2)cc1]

To a solution of (S)—N-(5-(2,5-dioxo-2,5-dihydro-1H-pyrrol-1-yl)pentyl)-N-(1-(4-(hydroxymethyl)phenylamino)-1-oxo-5-ureidopentan-2-yl)cyclobutane-1,1-dicarboxamide 10 in anhydrous DMF was added diisopropylethylamine (DIEA), followed by PNP carbonate (bis(4-nitrophenyl) carbonate). The reaction solution was stirred at room temperature (r.t.) for 4 hours and the mixture was purified by prep-HPLC to afford 12. LCMS (ESI): M+H⁺=736.29.

### Example 12 Preparation of MC-(CBDK-cit)-PAB-(dimethyl, fluoropipBOR)—PLA-1

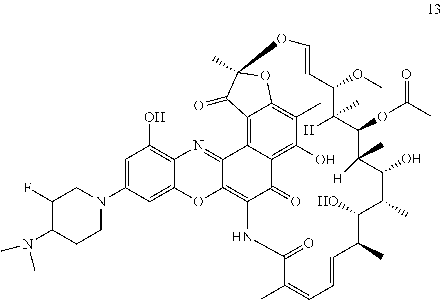 [SMILES: CO[C@H]1/C=C/O[C@@]2(C)Oc3c(C)c(O)c4c(=O)c(c5oc6cc(N7CCC(N(C)C)C(F)C7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C]

Following the procedure for PLA-2, (S)—N-(1-(4-(chloromethyl)phenylamino)-1-oxo-5-ureidopentan-2-yl)-N-(5-(2,5-dioxo-2,5-dihydro-1H-pyrrol-1-yl)pentyl)cyclobutane-1,1-dicarboxamide 11 and the fluorinated rifamycin-derivative, dimethylfluoropipBOR 13 (LCMS (ESI): M+H⁺=945.43) were reacted to form MC-(CBDK-cit)-PAB-(dimethyl, fluoropipBOR)—PLA-1, Table 2. LCMS (ESI): M+H⁺=1499.7 Example 13 Preparation of MC-(CBDK-cit)-PAB-(dimethylpipBOR)—PLA-2 (S)—N-(1-(4-(chloromethyl)phenylamino)-1-oxo-5-ureidopentan-2-yl)-N-(5-(2,5-dioxo-2,5-dihydro-1H-pyrrol-1-yl)pentyl)cyclobutane-1,1-dicarboxamide 11 (0.035 mmol) in DMF was cooled to 0° C. and dimethylpipBOR 6, (10 mg, 0.011 mmol) was added. The mixture was diluted with another 0.5 mL of DMF. Stirred open to air for 30 minutes. N,N-diisopropylethylamine (DIEA, 10 d_., 0.05 mmol) was added and the reaction stirred overnight open to air. By LC/MS, 50% of desired product was observed. An additional 0.2 eq N,N-diisopropylethylamine base was added while the reaction stirred open to air for another 6 hours until the reaction appeared to stop progressing. The reaction mixture was diluted with DMF and purified on HPLC (20-60% ACN/HCOOH in H₂O) to give MC-(CBDK-cit)-PAB-(dimethylpipBOR)—PLA-2, Table 2. LCMS (ESI): M+H⁺=1481.8, yield 31%.

### Example 14 Preparation of MC-((R)-thiophen-3-yl-CBDK-cit)-PAB-(dimethylpipBOR) (PLA-3)

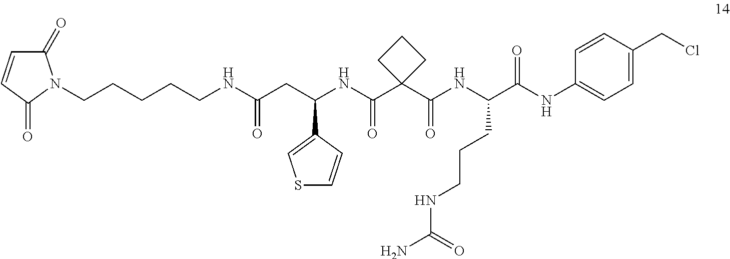 [SMILES: NC(=O)NCCC[C@H](NC(=O)C1(C(=O)N[C@H](CC(=O)NCCCCCN2C(=O)C=CC2=O)c2ccsc2)CCC1)C(=O)Nc1ccc(CCl)cc1]

Following the procedure for PLA-2, (N—((S)-1-(4-(chloromethyl)phenylamino)-1-oxo-5-ureidopentan-2-yl)-N—((R)-3-(5-(2,5-dioxo-2,5-dihydro-1H-pyrrol-1-yl)pentylamino)-3-oxo-1-(thiophen-3-yl)propyl)cyclobutane-1,1-dicarboxamide 14 (LCMS (ESI): M+H⁺=742.3) and dimethylpipBOR 6 were reacted to give MC-((R)-thiophen-3-yl-CBDK-cit)-PAB-(dimethylpipBOR) (PLA-3, Table 2). LCMS (ESI): M+H⁺.=1633.9

### Example 15 Preparation of MC-((S)-thiophen-3-yl-CBDK-cit)-PAB-(dimethylpipBOR) (PLA-4)

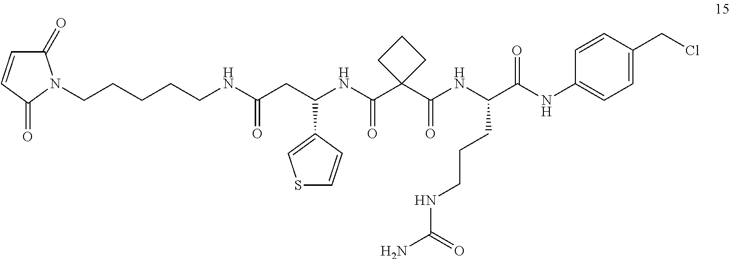

Following the procedure for PLA-2, (N—((R)-1-(4-(chloromethyl)phenylamino)-1-oxo-5-ureidopentan-2-yl)-N—((R)-3-(5-(2,5-dioxo-2,5-dihydro-1H-pyrrol-1-yl)pentylamino)-3-oxo-1-(thiophen-3-yl)propyl)cyclobutane-1,1-dicarboxamide 15 (LCMS (ESI): M+H⁺=742.3) and dimethylpipBOR 6 were reacted to give MC-((R)-thiophen-3-yl-CBDK-cit)-PAB-(dimethylpipBOR) (PLA-4, Table 2). LCMS (ESI): M+H⁺==1633.9

### Example 16 Preparation of MC-(CBDK-cit)-PABC-(pipBOR) (PLA-5)

Piperidyl benzoxazino rifamycin (pipBOR) 5 (15 mg, 0.0167 mmol), and then (S)-4-(2-(1-(5-(2,5-dioxo-2,5-dihydro-H-pyrrol-1-yl)pentylcarbamoyl)cyclobutanecarboxamido)-5-ureidopentanamido)benzyl 4-nitrophenyl carbonate 12 (12 mg, 0.0167 mmol) were weighed into a vial. Dimethylformamide, DMF (0.3 mL) was added, followed by diisopropylethylamine, DIEA (0.006 mL, 0.0334 mmol), and the reaction was allowed to stir at room temperature for 2 h. The reaction solution was directly purified by HPLC (30 to 70% MeCN/water+1% formic acid) to give MC-(CBDK-cit)-PABC-(pipBOR) (PLA-5, Table 2). LCMS (ESI): M+H⁺1496.5

### Example 17 Preparation of MC-(CBDK-cit)-PABC-(piperazBTR) (PLA-6)

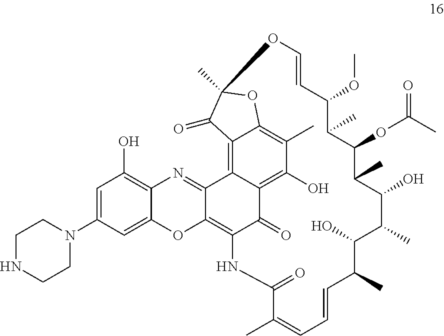 [SMILES: CO[C@H]1/C=C/O[C@@]2(C)Oc3c(C)c(O)c4c(=O)c(c5oc6cc(N7CCNCC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C]

Following the procedures for PLA-5, the piperidine rifamycin derivative, piperazBOR 16 (LCMS (ESI): M+H⁺1=885.4) and (S)-4-(2-(1-(5-(2,5-dioxo-2,5-dihydro-1H-pyrrol-1-yl)pentylcarbamoyl)cyclobutanecarboxamido)-5-ureidopentanamido)benzyl 4-nitrophenyl carbonate 12 were reacted to give MC-(CBDK-cit)-PABC-(piperazBTR) (PLA-6. Table 2). LCMS (ESI): M+H⁺=1482.5

### Example 18 Preparation of rF1 Antibody-Antibiotic Conjugates

Antibody-antibiotic conjugates (AAC) Table 3 were prepared by conjugating an rF1 antibody to a PML Linker-Antibiotic intermediate, including those from Table 2. Prior to conjugation, the rF1 antibodies were partially reduced with TCEP using standard methods in accordance with the methodology described in WO 2004/010957, the teachings of which are incorporated by reference for this purpose. The partially reduced antibodies were conjugated to the linker-antibiotic intermediate using standard methods in accordance with the methodology described, e.g., in Doronina et al. (2003) Nat. Biotechnol. 21:778-784 and US 2005/0238649 A1. Briefly, the partially reduced antibodies were combined with the linker-antibiotic intermediate to allow conjugation of the linker-antibiotic intermediate to reduced cysteine residues of the antibody. The conjugation reactions were quenched, and the AAC were purified. The antibiotic load (average number of antibiotic moieties per antibody) for each AAC was determined and was between about 1 to about 2 for the rF1 antibodies engineered with a single cysteine mutant site.

Reduction/Oxidation of ThioMabs for Conjugation: Full length, cysteine engineered monoclonal antibodies (ThioMabs—Junutula, et al., 2008b Nature Biotech., 26(8):925-932; Doman et al (2009) Blood 114(13):2721-2729; U.S. Pat. No. 7,521,541; U.S. Pat. No. 7,723,485; WO2009/052249, Shen et al (2012) Nature Biotech., 30(2):184-191; Junutula et al (2008) Jour of Immun. Methods 332:41-52) expressed in CHO cells were reduced with about a 20-40 fold excess of TCEP (tris(2-carboxyethyl)phosphine hydrochloride or DTT (dithiothreitol) in 50 mM Tris pH 7.5 with 2 mM EDTA for 3 hrs at 37° C. or overnight at room temperature. (Getz et al (1999) Anal. Biochem. Vol 273:73-80; Soltec Ventures, Beverly, Mass.). The reduced ThioMab was diluted and loaded onto a HiTrap S column in 10 mM sodium acetate, pH 5, and eluted with PBS containing 0.3M sodium chloride. Alternatively, the antibody was acidified by addition of 1/20th volume of 10% acetic acid, diluted with 10 mM succinate pH 5, loaded onto the column and then washed with 10 column volumes of succinate buffer. The column was eluted with 50 mM Tris pH7.5, 2 mM EDTA.

The eluted reduced ThioMab was treated with 15 fold molar excess of DHAA (dehydroascorbic acid) or 200 nM aqueous copper sulfate (CuSO4). Oxidation of the interchain disulfide bonds was complete in about three hours or more. Ambient air oxidation was also effective. The re-oxidized antibody was dialyzed into 20 mM sodium succinate pH 5, 150 mM NaCl, 2 mM EDTA and stored frozen at −20° C.

Conjugation of ThioMabs with linker-antibiotic intermediates: The deblocked, reoxidized, thio-antibodies (ThioMab) were reacted with 6-8 fold molar excess of the linker-antibiotic intermediate of Table 2 (from a DMSO stock at a concentration of 20 mM) in 50 mM Tris, pH 8, until the reaction was complete (16-24 hours) as determined by LC-MS analysis of the reaction mixture.

The crude antibody-antibiotic conjugates (AAC) were then applied to a cation exchange column after dilution with 20 mM sodium succinate, pH 5. The column was washed with at least 10 column volumes of 20 mM sodium succinate, pH 5, and the antibody was eluted with PBS. The AAC were formulated into 20 mM His/acetate, pH 5, with 240 mM sucrose using gel filtration columns. AAC were characterized by UV spectroscopy to determine protein concentration, analytical SEC (size-exclusion chromatography) for aggregation analysis and LC-MS before and after treatment with Lysine C endopeptidase.

Size exclusion chromatography was performed using a Shodex KW802.5 column in 0.2M potassium phosphate pH 6.2 with 0.25 mM potassium chloride and 15% IPA at a flow rate of 0.75 ml/min. Aggregation state of AAC was determined by integration of eluted peak area absorbance at 280 nm.

LC-MS analysis was performed using an Agilent QTOF 6520 ESI instrument. As an example, an AAC generated using this chemistry was treated with 1:500 w/w Endoproteinase Lys C (Promega) in Tris, pH 7.5, for 30 min at 37° C. The resulting cleavage fragments were loaded onto a 1000A, 8 um PLRP-S column heated to 80° C. and eluted with a gradient of 30% B to 40% B in 5 minutes. Mobile phase A: H₂O with 0.05% TFA. Mobile phase B: acetonitrile with 0.04% TFA. Flow rate: 0.5 ml/min. Protein elution was monitored by UV absorbance detection at 280 nm prior to electrospray ionization and MS analysis. Chromatographic resolution of the unconjugated Fc fragment, residual unconjugated Fab and antibiotic-Fab was usually achieved. The obtained m/z spectra were deconvoluted using Mass Hunter™ software (Agilent Technologies) to calculate the mass of the antibody fragments.

The AAC, 103 (AAR=1.9) thio-rF1-HC-121C, LC-V205C-MC-(CBDK-cit)-PAB-(dimethylpipBOR) was made using the rF1 L chain of SEQ ID NO. 9 containing the engineered Cys 205, and the rF1 H chain comprising SEQ ID NO. 10. The AAC 102 (AAR=3.9) thio-rF1-HC-121C, LC-V205C-MC-(CBDK-cit)-PAB-(dimethylpipBOR) was made using the rF1 L chain of SEQ ID NO. 9 in the preceding containing the engineered Cys 205, and the rF1 H chain comprising SEQ ID NO. 12 which contains the engineered Cys 114 (114 Kabat numbering is the same as 118 Eu numbering and 121 sequential numbering). The Cys engineered L and/or H chain was conjugated to the PML linker and rifamycin-type antibiotic as shown in Table 2.

### Example 19 In Vitro Efficacy of rF1-AACs

S. aureus (USA300 NRS384 strain) was incubated with various doses (100 ug/mL, 10 ug/mL, 1 ug/mL or 0.1 ug/mL) of an anti-S. aureus unconjugated antibody, 103 AAC loaded with 1.9 average antibiotic molecules per antibody (AAR2) or with 102 AAC loaded with 3.9 average antibiotic molecules per antibody (AAR4) for 1 hour to permit binding of the antibody to the bacteria. The resulting opsonized bacteria were fed to murine macrophages and incubated at 37° C. to permit phagocytosis (in vitro macrophage assay). After 2 hours, the infection mix was removed and replaced with normal growth media supplemented with 50 ug/mL of gentamycin to kill any remaining extracellular bacteria. The total number of surviving intracellular bacteria was determined 2 days later by plating serial dilutions of the macrophage lysates on Tryptic Soy Agar plates.

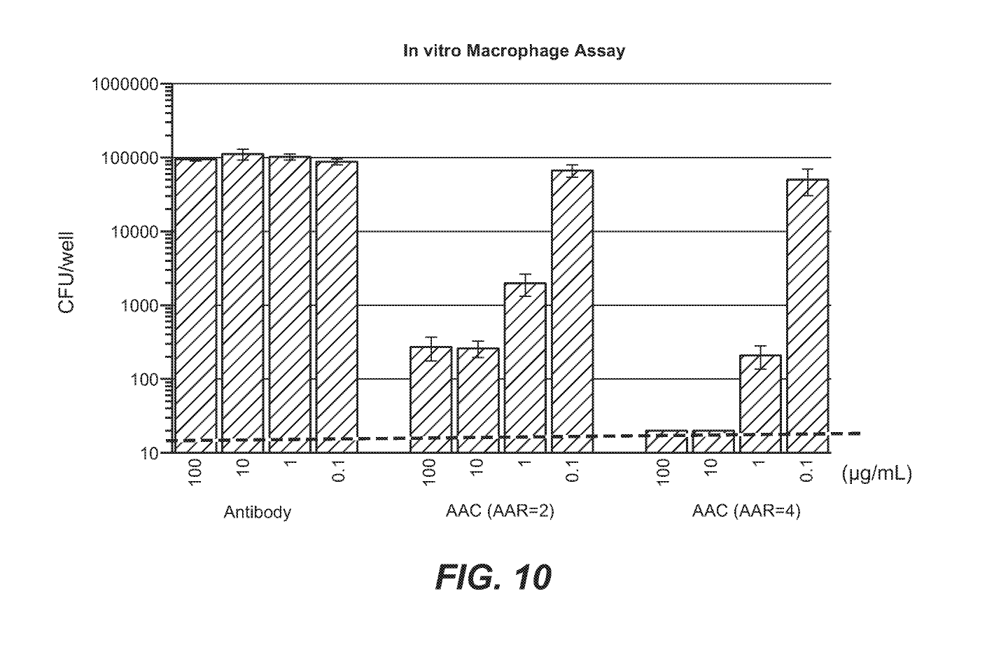

The results are shown in FIG. 10. Both of the AACs tested (AAR2 vs. AAR4) showed a similar dose response and yielded maximal killing at a dose of 10 ug/mL or above with partial to no killing at 1 ug/mL and below, suggesting that the dose response for the AAC is limited by the number of antibody binding sites on the bacterium. By loading 4 antibiotic molecules per antibody, bacterial killing by AACs and overall killing of bacteria was superior with the AAR4 AAC at all doses tested. At the highest dose tested, the 2DAR AAC reduced bacterial loads by 350-fold, whereas the 4AAR AAC reduced bacterial loads by more than 4,000-fold. (dashed line indicates the limit of detection for the assays shown).

This example demonstrates that rF1-AAC, 102 (AAR=3.9) and 103 (AAR=1.9) thio-rF1-HC-121C, LC-V205C-MC-(CBDK-cit)-PAB-(dimethylpipBOR) from Table 3 killed intracellular MRSA in a macrophage assay in vitro. The results are shown in FIG. 10.

### Example 20 In Vivo Efficacy of rF1-AACs

This example demonstrates that the rF1-AACs were effective in greatly reducing or eradicating intracellular S. aureus infections, in a murine intravenous infection model.

Peritonitis Model. 7 week old female A/J mice (Jackson Laboratories) are infected by peritoneal injection with 5×107 CFU of USA300. Mice are sacrificed 2 days post infection and the peritoneum is flushed with 5 mL of cold phosphate buffered saline solution (PBS). Kidneys are homogenized in 5 mL of PBS as described below for the intravenous infection model. Peritoneal washes are centrifuged for 5 minutes at 1,000 rpm at 4° C. in a table top centrifuge. The supernatant is collected as the extracellular bacteria and the cell pellet containing peritoneal cells is collected as the intracellular fraction. The cells are treated with 50 μg/mL of lysostaphin for 20 minutes at 37° C. to kill contaminating extracellular bacteria. Peritoneal cells are washed 3x in ice cold PBS to remove the lysostaphin prior to analysis. To count the number of intracellular CFUs, peritoneal cells are lysed in HB (Hanks Balanced Salt Solution supplemented with 10 mM HEPES and 0.1% Bovine Serum Albumin) with 0.1% Triton-X, and serial dilutions of the lysate are made in PBS with 0.05% tween-20.

Murine intravenous infection model. For studies involving competing human IgG (SCID IVIG model), CB17.SCID mice (Charles River Laboratories, Hollister, Calif.) were reconstituted with GammaGard S/D IGIV Immune Globulin (ASD Healthcare, Brooks Ky.) using a dosing regimen optimized to achieve constant serum levels of at least 10 mg/mL of human IgG in serum. IGIV was administered with an initial intravenous dose of 30 mg per mouse followed by a second dose of 15 mg/mouse by intraperitoneal injection after 6 hours, and subsequent daily dosing of 15 mg per mouse by intraperitoneal injection for 3 consecutive days.

Mice (n=8 for each of antibody or AAC) were infected 4 hours after the first dose of IGIV with 1×107 CFU of MRSA (USA300 NRS384 strain) diluted in phosphate buffered saline by intravenous injection. Infected mice were treated with 50 mg/kg of rF1 naked antibody, 103 AAC DAR2 or 102 AAC DAR4. Mice were given a single dose of AAC 26h post infection by intravenous injection, sacrificed on day 4 post infection, and kidneys and hearts were harvested in 5 mL of phosphate buffered saline. The tissue samples were homogenized using a GentleMACS Dissociator™ (Miltenyi Biotec, Auburn, Calif.). The total number of bacteria recovered per organ was determined by plating serial dilutions of the tissue homogenate in PBS 0.05% Tween on Tryptic Soy Agar with 5% defibrinated sheep blood.

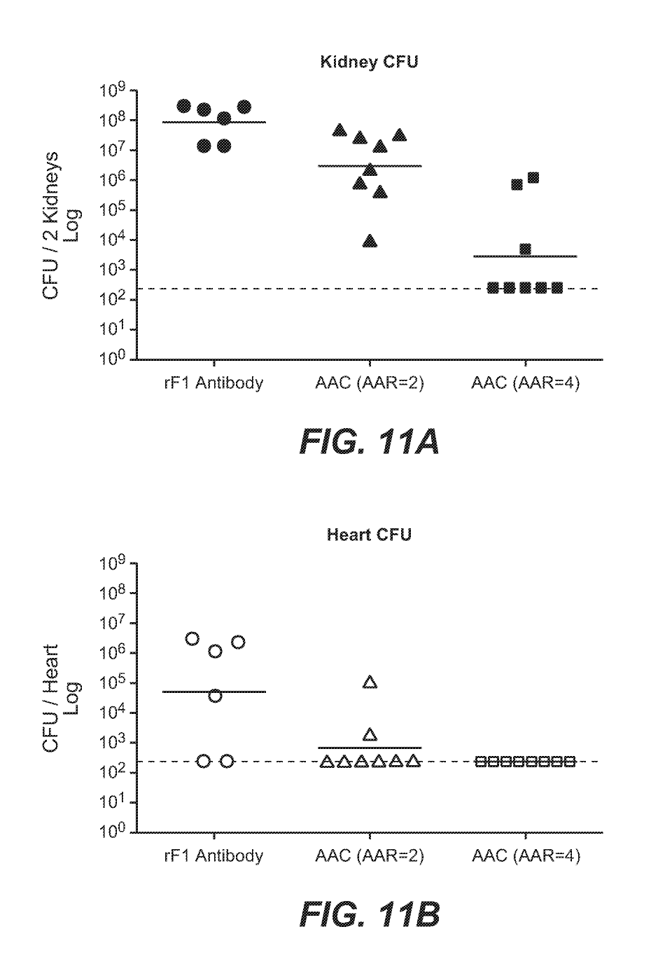

FIG. 11A shows the results of in vivo treatment with AACs on the bacterial load in the kidneys of the infected mice. Treatment with AAC containing 2 antibiotic molecules per antibody (DAR2) reduced bacterial load by approximately 30-fold and treatment with the AAC containing 4 antibiotic molecules per antibody (AAR4) reduced bacterial burdens by more than 30,000-fold.

FIG. 11B shows the results of in vivo treatment with AACs on the bacterial count in the heart. Treatment with AAC AAR2 reduced bacterial burdens by approximately 70-fold with 6 out of 8 mice having undetectable level of bacteria in hearts; treatment with the AAC DAR4 completely eradicated infection in hearts resulting in 8 out of 8 mice having undetectable levels of bacteria.

Although the foregoing invention has been described in some detail by way of illustration and example, for purposes of clarity of understanding, the descriptions and examples should not be construed as limiting the scope of the invention. All patents, patent applications, and references cited throughout the specification are expressly incorporated by reference.

1. An antibody-antibiotic conjugate compound comprising an anti-serine-aspartate repeat (SDR) antibody, wherein the antibody binds to Staphylococcus aureus, and covalently attached by a protease-cleavable, non-peptide linker to a rifamycin-type antibiotic.

2. The antibody-antibiotic conjugate compound of claim 1 having the formula:
Ab-(PML-abx)ₚ
wherein:
Ab is the anti-serine-aspartate repeat (SDR) antibody which is an rF1 antibody;
PML is the protease-cleavable, non-peptide linker having the formula:
-Str-PM-Y—
where Str is a stretcher unit; PM is a peptidomimetic unit, and Y is a spacer unit;
abx is the rifamycin-type antibiotic; and
p is an integer from 1 to 8.

3. (canceled)

4. The antibody-antibiotic conjugate compound of claim 2 wherein the rifamycin-type antibiotic comprises a quaternary amine attached to the protease-cleavable, non-peptide linker.

5. The antibody-antibiotic conjugate compound of claim 2 having Formula I: 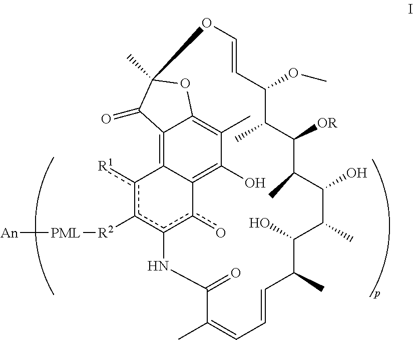
wherein:
the dashed lines indicate an optional bond;
R is H, C₁-C₁₂ alkyl, or C(O)CH₃;
R¹ is OH;
R² is CH═N-(heterocyclyl), wherein the heterocyclyl is optionally substituted with one or more groups independently selected from C(O)CH₃, C₁-C₁₂ alkyl, C₁-C₁₂ heteroaryl, C₂-C₂₀ heterocyclyl, C₆-C₂₀ aryl, and C₃-C₁₂ carbocyclyl;
or R¹ and R² form a five- or six-membered fused heteroaryl or heterocyclyl, and optionally forming a spiro or fused six-membered heteroaryl, heterocyclyl, aryl, or carbocyclyl ring, wherein the spiro or fused six-membered heteroaryl, heterocyclyl, aryl, or carbocyclyl ring is optionally substituted H, F, Cl, Br, I, C₁-C₁₂ alkyl, or OH;
PML is the protease-cleavable, non-peptide linker attached to R² or the fused heteroaryl or heterocyclyl formed by R¹ and R²; and
Ab is the rF1 antibody.

6. The antibody-antibiotic conjugate compound of claim 5 having the formula: 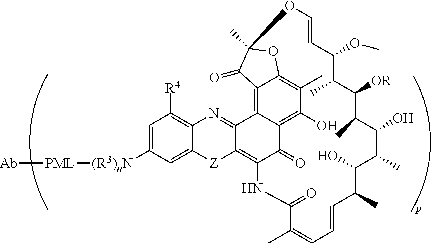
wherein
R³ is independently selected from H and C₁-C₁₂ alkyl;
n is 1 or 2;
R⁴ is selected from H, F, Cl, Br, I, C₁-C₁₂ alkyl, and OH; and
Z is selected from NH, N(C₁-C₁₂ alkyl), O and S.

7. The antibody-antibiotic conjugate compound of claim 1 selected from the formulas: 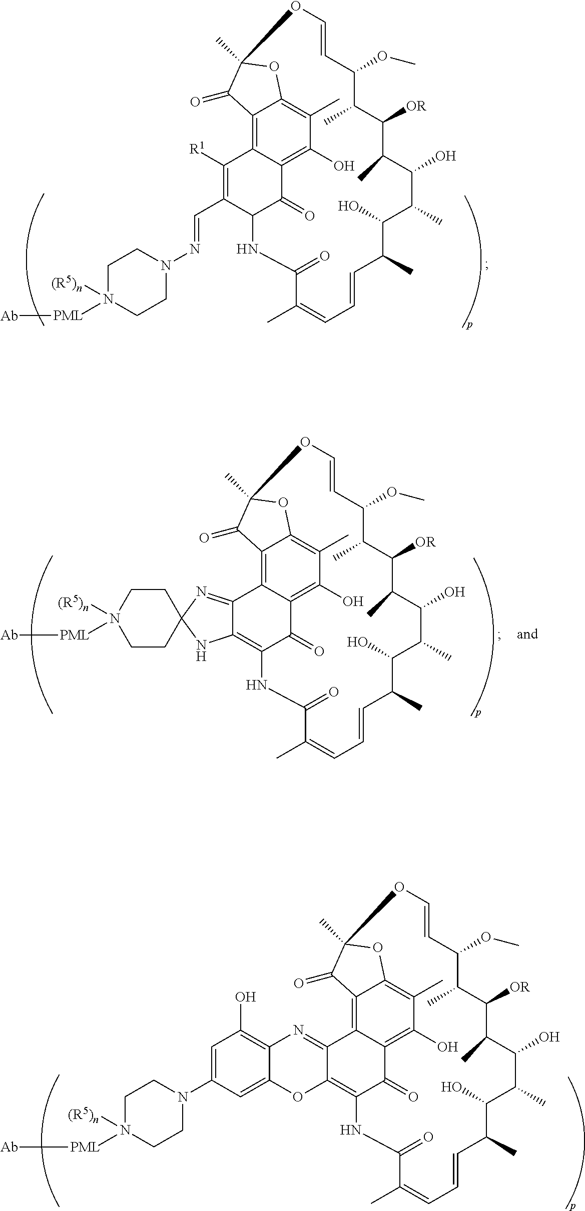
wherein
R⁵ is selected from H and C₁-C₁₂ alkyl; and
n is 0 or 1.

8. (canceled)

9. (canceled)

10. The antibody-antibiotic conjugate compound of claim 2 having the formula: 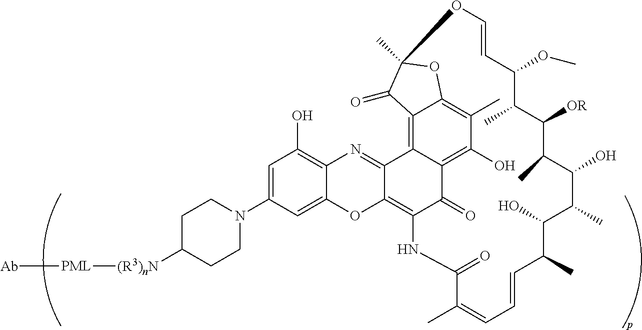
wherein
R³ is independently selected from H and C₁-C₁₂ alkyl; and
n is 1 or 2.

11. The antibody-antibiotic conjugate compound of claim 10 having the formula:

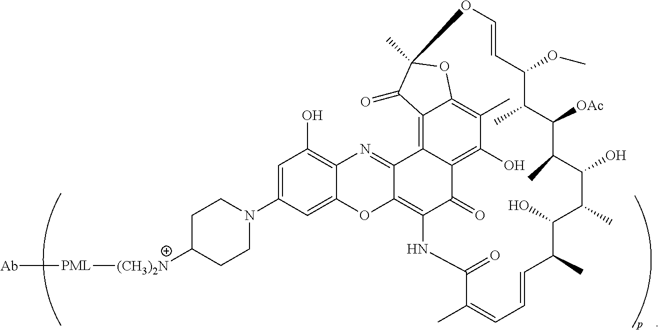

12. The antibody-antibiotic conjugate compound of claim 2 wherein Str has the formula: 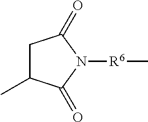
wherein R⁶ is selected from the group consisting of C₁-C₁₂ alkylene, C₁-C₁₂ alkylene-C(═O), C₁-C₁₂ alkylene-NH, (CH₂CH₂O)ᵣ, (CH₂CH₂O)ᵣ—C(═O), (CH₂CH₂O)ᵣ—CH₂, and C₁-C₁₂ alkylene-NHC(═O)CH₂CH(thiophen-3-yl), where r is an integer ranging from 1 to 10.

13. The antibody-antibiotic conjugate compound of claim 12 wherein R⁶ is (CH₂)5.

14. The antibody-antibiotic conjugate compound of claim 2 wherein PM has the formula: 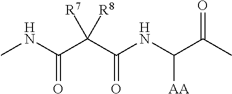
where R⁷ and R⁸ together form a C₃-C₇ cycloalkyl ring, and
AA is an amino acid side chain selected from H, —CH₃, —CH₂(C₆H₅), —CH₂CH₂CH₂CH₂NH₂, —CH₂CH₂CH₂NHC(NH)NH₂, —CHCH(CH₃)CH₃, and —CH₂CH₂CH₂NHC(O)NH₂.

15. The antibody-antibiotic conjugate compound of claim 2 wherein Y comprises para-aminobenzyl or para-aminobenzyloxycarbonyl.

16. The antibody-antibiotic conjugate compound of claim 2 having the formula:

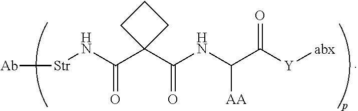

17. The antibody-antibiotic conjugate compound of claim 16 having the formula:

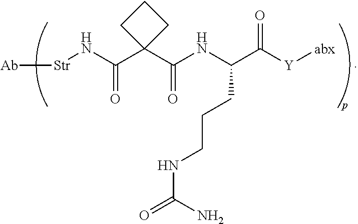

18. The antibody-antibiotic conjugate compound of claim 15 having the formula:

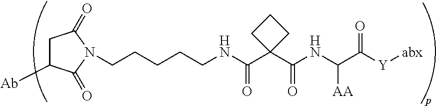

19. The antibody-antibiotic conjugate compound of claim 18 having the formula:

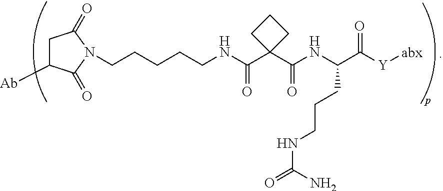

20. The antibody-antibiotic conjugate compound of claim 15 selected from the formulas:

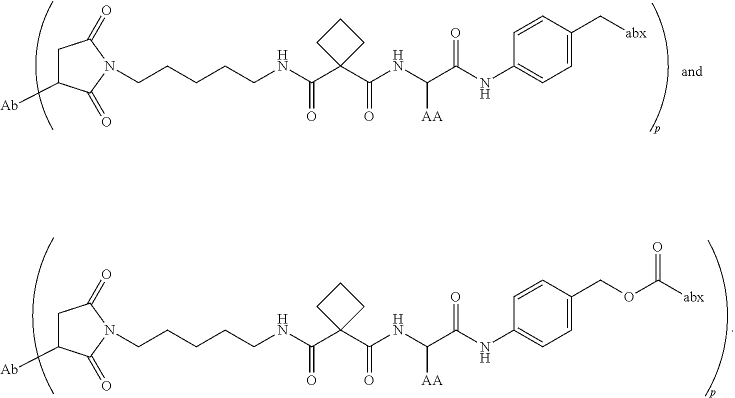

21. The antibody-antibiotic conjugate compound of claim 16 selected from the formulas:

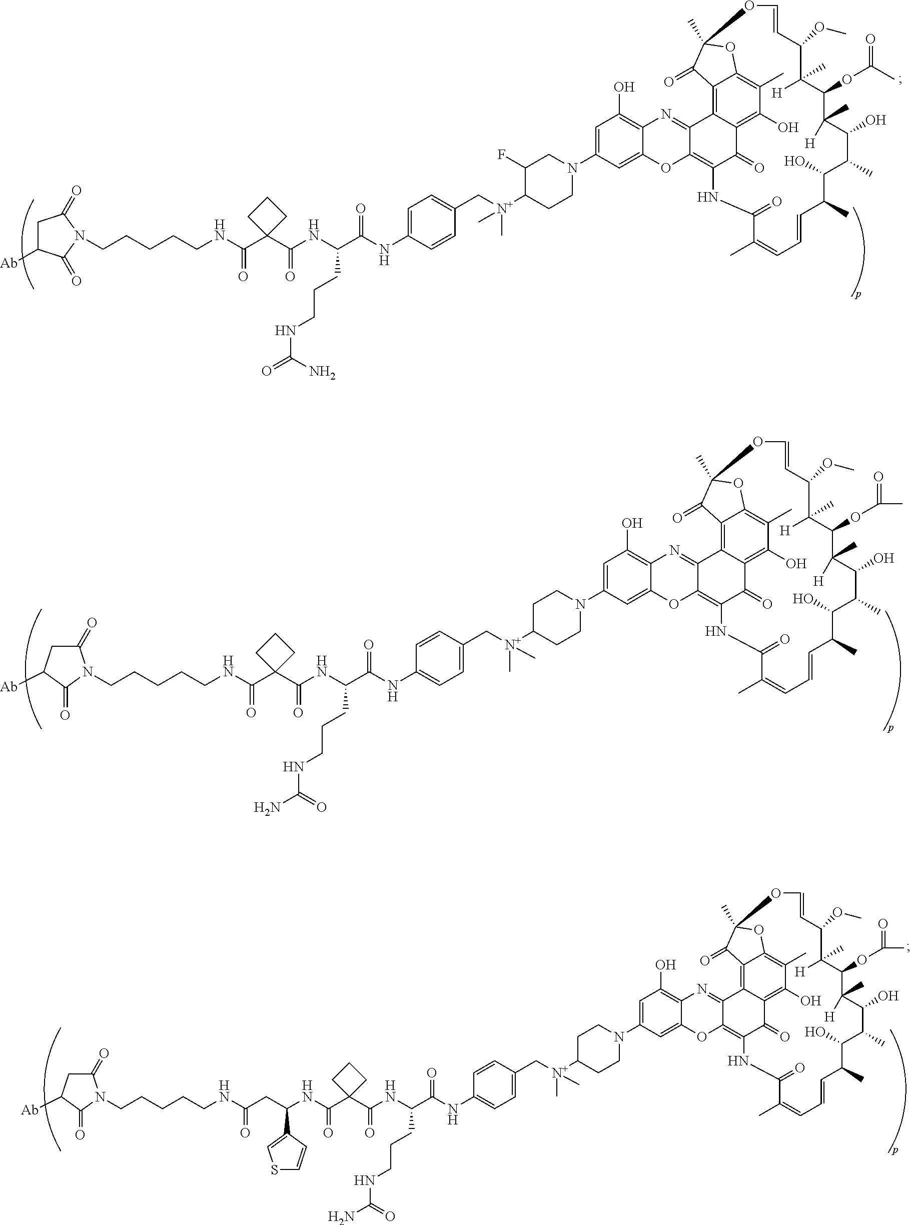

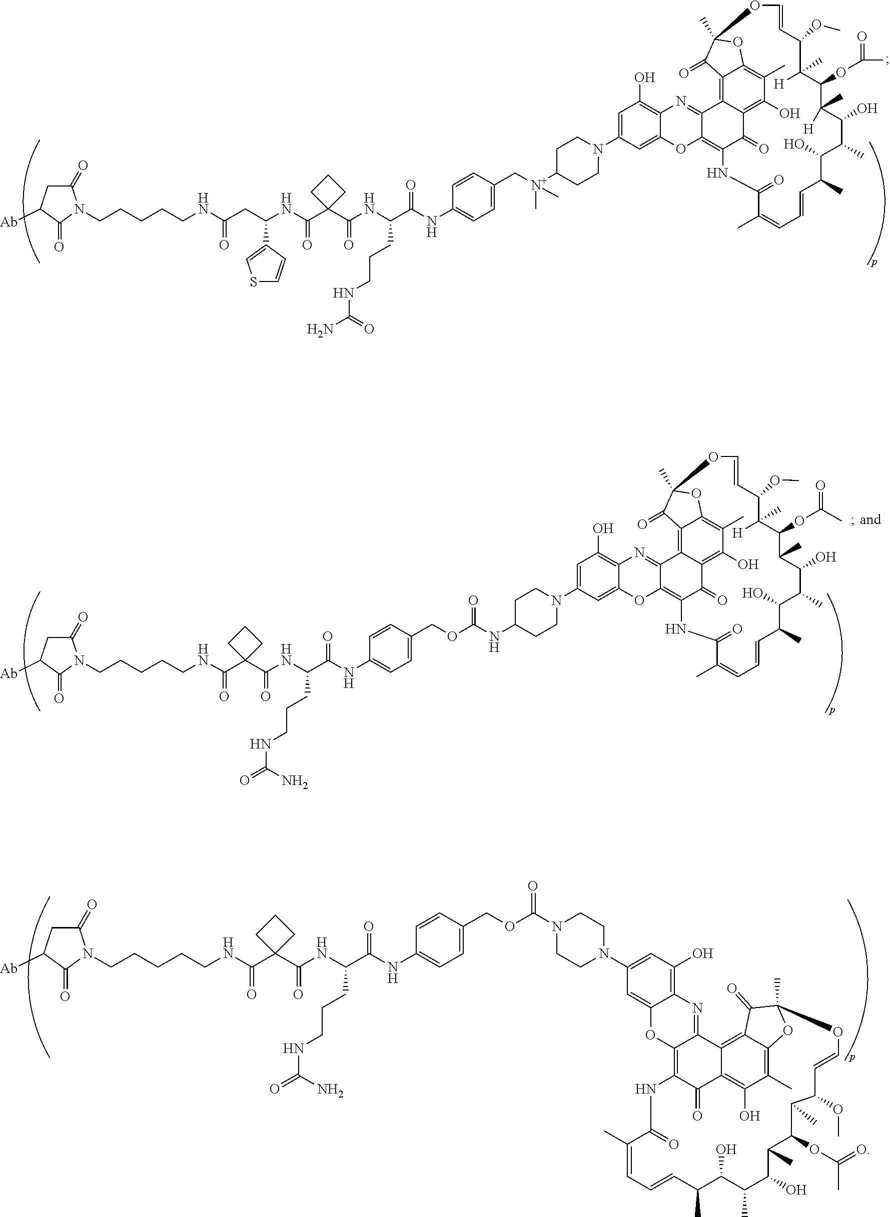

22. The antibody-antibiotic conjugate compound of claim 1, wherein the anti-SDR antibody is a rF1 antibody.

23. The antibody-antibiotic conjugate of claim 22, wherein the rF1 antibody comprises a light (L) chain and a heavy (H) chain, the L chain comprising CDR L1, CDR L2, and CDR L3 and the H chain comprising CDR H1, CDR H2 and CDR H3, wherein the CDR L1, CDR L2, and CDR L3 and CDR H1, CDR H2 and CDR H3 comprise the amino acid sequences of the CDRs of each of Abs F1, rF1, rF1.v1 and rF1.v6 (SEQ ID NO. 1-8), respectively, as shown in Table 4A and Table 4B.

24. The antibody-antibiotic conjugate of claim 22 wherein the rF1 antibody comprises a heavy chain variable region (VH), wherein the VH comprises at least 95% sequence identity over the length of the VH region of SEQ ID NO. 13.

25. The antibody-antibiotic conjugate compound of claim 24, wherein the VL comprises at least 95% sequence identity over the length of the VL region of SEQ ID NO. 14 or SEQ ID NO. 15.

26. (canceled)

27. (canceled)

28. A pharmaceutical composition comprising the antibody-antibiotic conjugate compound of claim 1, and a pharmaceutically acceptable carrier, glidant, diluent, or excipient.

29. A method of treating a Staphylococcus aureus infection in a patient comprising administering to the patient a therapeutically-effective amount of the antibody-antibiotic conjugate compound of claim 1.

30. (canceled)

31. The method of claim 30 wherein the patient is infected with Staphylococcus epidermidis.

32. The method of claim 29 wherein the antibody-antibiotic conjugate compound is administered to the patient at a dose in the range of about 50 mg/kg to 100 mg/kg.

33. The method of claim 29 wherein the patient is administered the antibody-antibiotic conjugate compound in conjunction with treatment with a second antibiotic.

34. A method of killing intracellular Staphylococcus aureus in the cells of a Staphylococcus aureus infected patient without killing the host cells by administering an antibody-antibiotic conjugate compound of claim 1.

35. A process for making the antibody-antibiotic conjugate compound of claim 1 comprising conjugating a rifamycin-type antibiotic to an rF1 antibody.

36. A kit for treating a Staphylococcus aureus infection, comprising:
a) the pharmaceutical composition of claim 23; and
b) instructions for use.

37-41. (canceled)


SEQUENCE LISTING (28 sequences)

SEQ ID NO: 1 - protein, 5 aa, Artificial Sequence
  source: /note=“Description of Artificial Sequence Synthetic peptide”
RFAMS

SEQ ID NO: 2 - protein, 16 aa, Artificial Sequence
  source: /note=“Description of Artificial Sequence Synthetic peptide”
SINNGNNPYYARSVQY

SEQ ID NO: 3 - protein, 12 aa, Artificial Sequence
  source: /note=“Description of Artificial Sequence Synthetic peptide”
DHPSSGWPTFDS

SEQ ID NO: 4 - protein, 11 aa, Artificial Sequence
  source: /note=“Description of Artificial Sequence Synthetic peptide”
RASENVGDWLA

SEQ ID NO: 5 - protein, 7 aa, Artificial Sequence
  source: /note=“Description of Artificial Sequence Synthetic peptide”
KTSILES

SEQ ID NO: 6 - protein, 9 aa, Artificial Sequence
  source: /note=“Description of Artificial Sequence Synthetic peptide”
QHYIRFPYT

SEQ ID NO: 7 - protein, 9 aa, Artificial Sequence
  source: /note=“Description of Artificial Sequence Synthetic peptide”
QHYMRFPYT

SEQ ID NO: 8 - protein, 16 aa, Artificial Sequence
  source: /note=“Description of Artificial Sequence Synthetic peptide”
SINSGNNPYYARSVQY

SEQ ID NO: 9 - protein, 214 aa, Artificial Sequence
  source: /note=“Description of Artificial Sequence Synthetic polypeptide”
DIQLTQSPSALPASVGDRVSITCRASENVGDWLAWYRQKPGKAPNLLIYKTSILESGVPSRFSGSGSGTEFTLTISSLQPDDFATYYCQHYMRFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 10 - protein, 449 aa, Artificial Sequence
  source: /note=“Description of Artificial Sequence Synthetic polypeptide”
EVQLVESGGGLVQPGGSLRLSCAASGFTLSRFAMSWVRQAPGRGLEWVASINSGNNPYYARSVQYRFTVSRDVSQNTVSLQMNNLRAEDSATYFCAKDHPSSGWPTFDSWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  source: /note=“Description of Artificial Sequence Synthetic polypeptide”
DIQLTQSPSALPASVGDRVSITCRASENVGDWLAWYRQKPGKAPNLLIYKTSILESGVPSRFSGSGSGTEFTLTISSLQPDDFATYYCQHYIRFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 12 - protein, 449 aa, Artificial Sequence
  source: /note=“Description of Artificial Sequence Synthetic polypeptide”
EVQLVESGGGLVQPGGSLRLSCAASGFTLSRFAMSWVRQAPGRGLEWVASINSGNNPYYARSVQYRFTVSRDVSQNTVSLQMNNLRAEDSATYFCAKDHPSSGWPTFDSWGPGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 120 aa, Artificial Sequence
  source: /note=“Description of Artificial Sequence Synthetic polypeptide”
EVQLVESGGGLVQPGGSLRLSCAASGFTLSRFAMSWVRQAPGRGLEWVASINSGNNPYYARSVQYRFTVSRDVSQNTVSLQMNNLRAEDSATYFCAKDHPSSGWPTFDSWGPGTLVTVSS

SEQ ID NO: 14 - protein, 110 aa, Artificial Sequence
  source: /note=“Description of Artificial Sequence Synthetic polypeptide”
DIQLTQSPSALPASVGDRVSITCRASENVGDWLAWYRQKPGKAPNLLIYKTSILESGVPSRFSGSGSGTEFTLTISSLQPDDFATYYCQHYMRFPYTFGQGTKVEIKRTV

SEQ ID NO: 15 - protein, 110 aa, Artificial Sequence
  source: /note=“Description of Artificial Sequence Synthetic polypeptide”
DIQLTQSPSALPASVGDRVSITCRASENVGDWLAWYRQKPGKAPNLLIYKTSILESGVPSRFSGSGSGTEFTLTISSLQPDDFATYYCQHYIRFPYTFGQGTKVEIKRTV

SEQ ID NO: 16 - protein, 30 aa, Artificial Sequence
  source: /note=“Description of Artificial Sequence Synthetic polypeptide”
EVQLVESGGGLVQPGGSLRLSCAASGFTLS

SEQ ID NO: 17 - protein, 14 aa, Artificial Sequence
  source: /note=“Description of Artificial Sequence Synthetic peptide”
WVRQAPGRGLEWVA

SEQ ID NO: 18 - protein, 32 aa, Artificial Sequence
  source: /note=“Description of Artificial Sequence Synthetic polypeptide”
RFTVSRDVSQNTVSLQMNNLRAEDSATYFCAK

SEQ ID NO: 19 - protein, 11 aa, Artificial Sequence
  source: /note=“Description of Artificial Sequence Synthetic peptide”
WGPGTLVTVSS

SEQ ID NO: 20 - protein, 23 aa, Artificial Sequence
  source: /note=“Description of Artificial Sequence Synthetic peptide”
DIQLTQSPSALPASVGDRVSITC

SEQ ID NO: 21 - protein, 15 aa, Artificial Sequence
  source: /note=“Description of Artificial Sequence Synthetic peptide”
WYRQKPGKAPNLLIY

SEQ ID NO: 22 - protein, 32 aa, Artificial Sequence
  source: /note=“Description of Artificial Sequence Synthetic polypeptide”
GVPSRFSGSGSGTEFTLTISSLQPDDFATYYC

SEQ ID NO: 23 - protein, 13 aa, Artificial Sequence
  source: /note=“Description of Artificial Sequence Synthetic peptide”
FGQGTKVEIKRTV

SEQ ID NO: 24 - protein, 550 aa, Staphylococcus epidermidis
  VARIANT (51)..(550): /replace=“ ”
  MISC_FEATURE (1)..(550): /note=“This sequence may encompass 25 to 275 ′Ser-Asp′ repeating units, wherein some positions may be absent”
  misc_feature (1)..(550): /note=“Variant residues given in the sequence have no preference with respect to those in the annotations for variant positions”
SDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSD

SEQ ID NO: 25 - protein, 5 aa, Staphylococcus epidermidis
  MOD_RES (3)..(3): Any amino acid
LPXTG

SEQ ID NO: 26 - protein, 9 aa, Artificial Sequence
  source: /note=“Description of Artificial Sequence Synthetic peptide”
  VARIANT (4)..(4): /replace=“Met”
  misc_feature (1)..(9): /note=“Variant residues given in the sequence have no preference with respect to those in the annotations for variant positions”
QHYIRFPYT

SEQ ID NO: 27 - protein, 8 aa, Staphylococcus sp.
SDSDSDSD

SEQ ID NO: 28 - protein, 6 aa, Staphylococcus sp.
SDSDSD